(19) United States
(12) Patent Application Publication
(10) Pub. No.: US 2026/0258149 A1
(43) Pub. Date: Sep. 3, 2026

(54) HUMANISED ANTIBODIES OR FUNCTIONAL FRAGMENTS THEREOF AGAINST TUMOUR ANTIGENS

(71) Applicant: BioNTech SE, Mainz (DE)

(72) Inventors: Nuno José Prego Ramos, Ramada (PT); Natale Mariangela, Lisboa (PT); Leonardo Chicaybam Peixoto, Oeiras (PT)

(21) Appl. No.: 18/878,747

(22) PCT Filed: Jun. 26, 2023
(86) PCT No.: PCT/PT2023/050016

§ 371 Date: Dec. 24, 2024

(30) Foreign Application Priority Data
Jun. 24, 2022 (GB) 2209332.2

(51) Int. Cl. C07K 16/30; A61K 47/68; A61K 48/00; A61P 35/00; G01N 33/575

(52) U.S. Cl. CPC C07K 16/30; A61K 47/6851; A61K 48/005; A61P 35/00; G01N 33/5759; C07K 2317/24; C07K 2317/92

(57) ABSTRACT

There is provided an antibody or fragment thereof, wherein the antibody comprises: (a) a heavy chain variable region (VH) wherein the VH comprises complementarity determining regions (CDRs) selected from the group consisting of: (i) H-CDR1, H-CDR2 and H-CDR3 as shown in any one SEQ ID NOs. 1 to 24 or 49 to 88 respectively and/or, (b) a light chain variable region (VL) wherein the VL comprises complementarity determining regions (CDRs) selected from the group consisting of: (i) L-CDR1, L-CDR2, and L-CDR3 as shown in any one of SEQ ID NOs. 25 to 48 or 89 to 128 respectively. Preferably, both a VH and a VL chains are used. The antibodies and fragments thereof have utility in treating cancer.

### TECHNICAL FIELD

The present invention relates to humanised antibodies or functional antibody fragments thereof, or probes thereof, directed against a group of antigens identified in cancer.

### STATE OF THE ART

The sialyl Tn (STn) is a short O-glycan antigen, a disaccharide consisting of sialic acid linked to N-acetylgalactosamine, i.e. Neu5Acα-2, 6GalNAc, O-linked to Serine or Threonine amino acid residues, in alpha configuration through the GalNAc residue, in a polypeptide chain. This truncated glycan has been detected with different frequencies in different types of carcinomas (Julian, Videira, & Delannoy 2012), while absent in normal healthy tissues. In addition, STn is targeted for metastatic, drug resistant and highly malignant tumours. In this type of tumours, their characteristics are the following:

1) Association of STn expression with early and metastatic cancer cells (Okasaki et al., 2012).

2) Correlation of increased STn expression in patients with poor prognosis, reduced overall survival, and lack of response to chemotherapy (Choi et al., 2000)

3) Evasion of immune-cells surveillance (Carrascal et al., 2014)

Alpha-2, 6 sialic acids are typically truncated cancer biomarkers. Short alpha 2,6 sialylated O-glycans are overexpressed in several types of cancer. They are generally involved in cancer progression and metastasis. In addition, truncate glycans have been reported to contribute to immune evasion through their recognition by a number of immune receptors, such as the sialic acid binding proteins (Siglecs) (Crocke, Paulson, & Varkl, 2007; Micoll et al., 2003).

The present invention provides further antibodies, functional antibody fragments thereof, or probes thereof, that bind specifically to these cancer biomarkers. Besides the specific identification of tumour cells, these antibodies also have the potential to block the recognition of such ligands by host cell receptors involved in the mechanism underlying tumour progression, including immune tolerance.

A number of antibodies have been approved to treat cancer patients (see, for example: https://www.cancer.org), and further details of these, and further background to the present invention can be found in WO2019/147152.

WO2019/147152 describes nucleotide sequences encoding a monoclonal antibody (mAb) against the STn and a group of glycans terminated by alpha-2, 6-linked sialic acids. These antigens are short-chain glycans that are overexpressed in cancer but not expressed by normal cells.

### SUMMARY OF THE INVENTION

In a broad aspect, according to the present invention, there is provided an antibody or fragment thereof, or probe thereof, wherein the antibody or fragment or probe comprises:
(a) a heavy chain variable region (VH) wherein the VH comprises complementarity determining regions (CDRs) selected from the group consisting of:
(i) H-CDR1, H-CDR2 and H-CDR3 as shown in any one SEQ ID NOs. 1 to 24 or 49 to 88 respectively
and/or,
(b) a light chain variable region (VL) wherein the VL comprises complementarity determining regions (CDRs) selected from the group consisting of:
(i) L-CDR1, L-CDR2, and L-CDR3 as shown in any one of SEQ ID NOs. 25 to 48 or 89 to 128 respectively.

The fragment may be a functional antibody fragment of the antibody disclosed—that is, retain the ability to bind antigen. The antibody, fragment or probe described herein is able to specifically bind to the sialyl Tn (STn) antigen. Suitably, the antibody, fragment or probe is a humanised antibody, fragment or probe, and thus suitable for application in human patients. The antibody, fragment or probe may for example be an affinity-matured humanised antibody, fragment or probe.

In one aspect, the antibody or fragment thereof may comprise a heavy chain variable region (VH) and a light chain variable region (VL). Constant regions may also be provided, as will be understood.

Thus, in one aspect, the present invention essentially provides engineered antibodies with amino acid changes in the chimeric L2A5 framework regions described in WO2019147152 in order to humanize it. The primary scope of humanization is to engineer antibodies from non-human species, whose protein sequences have been modified to increase their similarity to antibodies normally produced in humans. The present inventors have discovered and produced humanized L2A5 variants, as disclosed in the present application, while retaining binding and specificity to STn whilst showing decreased immunogenicity.

In a further aspect, the invention also describes additional engineered antibodies based on a humanized variant, termed V1 in this application. Through a process of affinity maturation, different point mutations were introduced in the CDRs which enabled the present inventors to generate variants with higher binding affinity to STn. As disclosed in this patent application, several antibody variants have shown increased binding to STn+ cell lines and BSM mucin, with no relevant binding to other glycans.

The present application describes the generation of humanized antibodies, recognizing STn with moderate to high affinity and high specificity—characteristics which generally cannot be predicted in advance. Humanized clones also potentially have decreased immunogenicity, which decreases the chance of human anti-drug antibodies (HAMA) response, increases the therapeutic window and improves the antibody PK profile.

Furthermore, the present application describes the development of high affinity humanized anti-STn antibodies through a process of affinity maturation. For certain therapeutic applications, it is desirable to have antibodies with high affinity to the target antigen, thus improving the antitumor response.

The present inventors have now found a way to improve the antibody affinity and binding to the target, Sialyl Tn (STn), by the generation of new antibodies. These are related to the clone described in patent WO2019147152A1. In particular, the present inventors have provided a number of different humanised antibody clones, including the humanized clone referred to herein with the acronym of V1. Using in particular this humanized V1 clone, the present inventors have also now provided a series of new and different antibodies resulting from an affinity maturation process. These new clones or variants possess differences in the amino acid sequences and an increase in affinity and binding to the target Sialyl Tn (STn), thus substantially improving over the known antibodies.

The present application thus describes high affinity humanized anti-STn antibodies obtained through a process of affinity maturation. For certain applications, it is highly desirable to have antibodies with high affinity to the target antigen, improving the antitumor response. For instance, this may allow for:
antibody-drug conjugates or radioimmunoconjugates with increased tumour uptake and antitumor function.
fine-tuning of bispecific T cell engagers and CAR receptors to induce the desired T cell activation level.
improved blocking of STn in vivo, leading to antitumor responses by restoring the function of immune cells.

### BRIEF DESCRIPTION OF FIGURES

FIG. 1 shows the sequence ID numbers assigned to the variants disclosed herein, which are reflected in the accompanying sequence listing.

FIG. 2 shows the amino acid sequences of the variable chains (VH and VL) of certain humanised variants disclosed herein.

FIG. 3a shows the amino acid sequence alignment comparing the heavy chain (VH) of the parental antibody (L2A5) to certain humanized variants described herein, wherein:

* (Asterix) positions with a single, fully conserved residue.

: (colon) positions with conservation between amino acid groups of similar properties.

. (period) positions with conservation between amino acid groups of weakly similar properties.

FIG. 3b shows the amino acid sequence alignment comparing the light chain (VL) of the parental antibody (L2A5) to certain humanized variants described herein, wherein:

* (Asterix) positions with a single, fully conserved residue.

: (colon) positions with conservation between amino acid groups of similar properties.

. (period) positions with conservation between amino acid groups of weakly similar properties.

FIG. 4 shows the amino acid sequence of the VH heavy chain of humanised variants disclosed herein. The H-CDR1, H-CDR2 and H-CDR3 sequences are highlighted in bold.

FIG. 5 shows the amino acid sequence of the V_{L} light chain of humanised antibody variants disclosed herein. The L-CDR1, L-CDR2 and L-CDR3 sequences are highlighted in bold.

FIG. 6a shows the amino acid sequence of the V_{H} heavy chain of affinity-matured variants disclosed herein. The H-CDR1, H-CDR2 and H-CDR3 sequences are highlighted in bold.

FIG. 6b shows the amino acid sequence of the V_{L} light chain of affinity-matured variants disclosed herein. The L-CDR1, L-CDR2 and L-CDR3 sequences are highlighted in bold.

FIG. 7 shows the amino acid sequences of the variable chains (VH and VL) of the affinity-matured variants disclosed herein, together with the clone variant name and their corresponding sequence identity no., as reflected in the accompanying sequence listing.

FIG. 8 shows normalized EC50 using humanized Abs clones V1, V2 and V3, percentage of dose response effect (EC50) of V1-3 humanized clones screened against BSM mucin by ELISA. EC50 was plotted as percentage of dose response effect (y-axis) versus the antibody dose in log scale (x-axis). Clone V2 has a lower EC50 compared to the V1 and V3 clones. EC50 values: V1 0,3238 to 0,4573; V2 0,06649 to 0,1365; V3 0,3440 to 0,4490.

FIG. 9 shows binding of humanized clones to different STn+ cancer cell lines. The three variants shown (V1, V2 and V3) maintained high binding to different cell lines expressing STn.

FIG. 10 shows NMR analysis of the points of contact/interaction between the antibody and glycan molecules, using humanized variant 6 (V6), it was observed that the antibody binds mostly to the sialic acid portion of STn (orange circles), but also interacts with the GalNac portion (lower right side of the scheme).

FIG. 11 shows how Surface Plasmon Resonance (SPR) can be used to measure the affinity and kinetic parameters of different anti-STn antibodies. BSA-STn was immobilized to the chip and the antibodies were injected over the surface as association phase. CellmAbs CBS humanized clones (V1-V3) showed a high affinity towards BSA-STn, with KD ranging from 29 nM to 39 nM. No interaction was detected between the positive control clone and BSA-STn.

FIGS. 12A and 12B shows results of glycan array analysis, using different immobilized glycan probes, to evaluate the binding specificity and the binding intensity of antibodies to a panel of α2-6 sialylated glycans. A shows humanized mAbs V1-V3 have increased binding to STn-Ser when compared to “positive control”. B shows retained binding to STn-Ser of humanized clones V1 and affinity matured clone V64 compared to chimeric L2A5 antibody. In addition, clone V1 and V64 showed improved binding to STn-Thr compared to parental Chimeric L2A5 antibody.

FIG. 13A to D show the results from an in silico immunogenicity analysis, affinity value prediction (y-axis) of VH and VL region of V1 and V2 antibody clones, respectively compared to the Chimeric/Parental L2A5 Ab, at different positions and using different protein sequence (x-axis). V1 and V2 reported decrease immunogenicity values compared to chimeric L2A5.

FIG. 14 shows results from antibody internalization-assay analysis using humanized antibody variants (V2, V1) using MDA-MB231 breast cancer cell line, overexpressing STn transgene, using humanized Abs variants (V1-2) compared to control IgG and 3F1, commercially available Abs. Antibody internalization is reported as signal-to-background ratio MFI (y-axis), normalized to untreated cells at 2-6 and 16 hours (x-axis).

FIG. 15 shows representative IHC staining results using humanized V1, V2 and V3 antibody-clones in colon carcinoma and different metastatic tissues. The scale bar represents 50 μm.

FIG. 16 shows EC50 results for certain affinity-matured antibody variants binding property to Bovine Submaxillary Mucin (BSM) using ELISA assay and EC50 analysis. Binding to BSM is shown as Absorbance (OD) on the y-axis at different antibody concentrations (u (g/mL) on the x-axis. Clones tested included new variants with point mutations in the CDR (selected after the affinity maturation process).

FIG. 17 shows the binding profile for certain affinity-matured variants using different cancer cell lines, presenting different STn expression levels. Each antibody was titrated using a 7-point concentration (μg/mL) curve and the binding intensity was measured by flow cytometry using a secondary antibody conjugated to a fluorophore.

FIG. 18 shows the EC50 for certain affinity-matured variants based on binding results obtained in MDA-MB-231 STn breast cancer cell lines, the EC50 for each antibody was calculated. This table summarizes the EC50 obtained for each clone as well as the maximum MFI detected.

FIG. 19 shows binding results to different types of glycans, using different sialylated probes for certain affinity-matured variants. Binding reported as fluorescent intensity (y-axis) of affinity-matured clones using a glycan array with different sialylated probes (x-axis). Affinity-matured clones retained specificity to STn-serine (probe3) and STn-threonine (probe4).

FIG. 20a shows the sequence ID No and amino acid sequence of the VH heavy chain of humanised V1 variants disclosed herein. The H-CDR1, H-CDR2 and H-CDR3 sequences are highlighted in bold.

FIG. 20b shows the sequence ID No and amino acid sequence of the VL light chain of humanised V1 variants disclosed herein. The L-CDR1, L-CDR2 and L-CDR3 sequences are highlighted in bold.

It will be understood that monoclonal antibodies (mAbs) refers to an antibody that is produced by a single B cell clone. MAbs can be also produced by an hybridoma, which is a hybrid between a B cell and myeloma cell, or cell lines that express recombinant DNA coding for the immunoglobulin heavy and light chain, and therefore will produce a single and specific antibody.

The antibodies may be expressed to the extracellular milieu and then purified from there.

The specificity of an antibody is its ability to react with one antigen or a group of antigens that share a certain epitope. An epitope, also known as antigenic determinant, is the part of an antigen that is recognised by the antibody.

An antibody belongs to the immunoglobulin class of proteins, and it is typically an assembling of two identical heavy chains (around 50-70 kDa) and too identical light chains (around 25 kDa). In the amino-terminal of each heavy or light chain there is a sequence: of 100-130 amino acids that code for the variable region. In the carboxyl-terminal of each heavy or light chain there is a sequence that codes the constant region. Typically, each antibody binds the same antigen, i.e. is bivalent.

The antigen-binding fragment (Fab) is the antibody fragment that binds to antigens. Each Fab is composed of one constant and one variable domain from each heavy and light chain of the antibody. The Fragment crystallisable (Fc) region is composed of 2 or 3 domains of the carboxy-terminal of the two heavy chains. While the Fab ensures binding to the antigen, the Fc region ensures that each antibody generates an effector immune response. The Fc region binds to various cell receptors, such as Fc receptors, and other molecules, such as complement proteins, mediating different physiological effects including opsonization to facilitate phagocytosis by phagocytes, cell lysis by natural killer cells, and degranulation of mast cells, basophils and eosinophils.

The term “variable domain” or “variable region” is the amino-terminal part of the light or heavy chains of an antibody that interacts with the antigen. It typically has a length of about 120 to 130 amino acids in the heavy chain and typically about 100 to 110 amino acids in the light chain. The sequences of each of the variable regions are substantially varied, particularly in the complementary determining regions (CDRs) responsible for the interaction with the specific antigen. The CDRs are flanked by less varied framework regions (FR). There are typically three CDRs in each of the light and heavy chains. Thus, for example, CDRs L1, L2, and L3 are within the light chain, and CDRs H1, H2 and H3 are within the heavy chain.

The expression “functional antibody fragment or probe” suitably refers to a part of the antibody that includes the variable region of the heavy and the light chain of the antibody or includes either the variable region of the heavy or the variable region of the light chain of the antibody. For example, a functional antibody fragment or probe retains most or all the binding activity of the initial antibody from which the fragment or probe is derived. Such functional antibody fragments or probes can for example include the single chain Fv (scFv), diabody, triabody, tetra-body and mini-body.

It will be appreciated that the term fragment as used herein in particular relates to fragments of antibodies specifically as described and these form an important aspect of the present disclosure. In this way, a monoclonal or recombinant antibody as provided by the present disclosure may for example be provided as any of the following fragments: (i) the Fab fragment consisting of VL, VH, CL and CH1 domains; (ii) the Fd fragment consisting of the VH and CH1 domains; (iii) the Fv fragment consisting of the VL and VH domains; (iv) the dAb fragment which consists of a VH domain; (v) the isolated CDR regions; (vi) F(ab′)2 fragments, a bivalent fragment comprising two linked Fab fragments; and (vii) single chain Fv molecules (scFv), wherein a VH domain and a VL domain are linked by a peptide linker which allows the two domains to associate to form an antigen binding site.

Alternatively, as will be understood, an antibody according to the present disclosure may comprise a whole IgG antibody, whereby the antibody includes variable and constant regions.

The term “nucleotide sequences” refers to a sequence of nucleotides of any length, either deoxy ribonucleotides or ribonucleotides or their analogues thereof.

As will be understood, nucleotide sequences can be transcribed to produce mRNA, which is then translated into a polypeptide and/or a fragment thereof.

Further aspects of the invention will now be described. In one preferred aspect, an antibody or functional antibody fragment or probe thereof is provided, wherein the antibody or fragment or probe comprises one of the following pairs of heavy chain CDRs and light chain CDRs:
H-CDR1, H-CDR2 and H-CDR3 as shown in SEQ ID NO. 1 paired with L-CDR1, L-CDR2, and L-CDR3 as shown in SEQ ID NO. 25;
H-CDR1, H-CDR2 and H-CDR3 as shown in SEQ ID NO. 2 paired with L-CDR1, L-CDR2, and L-CDR3 as shown in SEQ ID NO. 26;
H-CDR1, H-CDR2 and H-CDR3 as shown in SEQ ID NO. 3 paired with L-CDR1, L-CDR2, and L-CDR3 as shown in SEQ ID NO. 27;
H-CDR1, H-CDR2 and H-CDR3 as shown in SEQ ID NO. 4 paired with L-CDR1, L-CDR2, and L-CDR3 as shown in SEQ ID NO. 28;
H-CDR1, H-CDR2 and H-CDR3 as shown in SEQ ID NO. 5 paired with L-CDR1, L-CDR2, and L-CDR3 as shown in SEQ ID NO. 29;
H-CDR1, H-CDR2 and H-CDR3 as shown in SEQ ID NO. 6 paired with L-CDR1, L-CDR2, and L-CDR3 as shown in SEQ ID NO. 30;
H-CDR1, H-CDR2 and H-CDR3 as shown in SEQ ID NO. 7 paired with L-CDR1, L-CDR2, and L-CDR3 as shown in SEQ ID NO. 31;
H-CDR1, H-CDR2 and H-CDR3 as shown in SEQ ID NO. 8 paired with L-CDR1, L-CDR2, and L-CDR3 as shown in SEQ ID NO. 32;
H-CDR1, H-CDR2 and H-CDR3 as shown in SEQ ID NO. 9 paired with L-CDR1, L-CDR2, and L-CDR3 as shown in SEQ ID NO. 33;
H-CDR1, H-CDR2 and H-CDR3 as shown in SEQ ID NO. 10 paired with L-CDR1, L-CDR2, and L-CDR3 as shown in SEQ ID NO. 34;
H-CDR1, H-CDR2 and H-CDR3 as shown in SEQ ID NO. 11 paired with L-CDR1, L-CDR2, and L-CDR3 as shown in SEQ ID NO. 35;
H-CDR1, H-CDR2 and H-CDR3 as shown in SEQ ID NO. 12 paired with L-CDR1, L-CDR2, and L-CDR3 as shown in SEQ ID NO. 36;
H-CDR1, H-CDR2 and H-CDR3 as shown in SEQ ID NO. 13 paired with L-CDR1, L-CDR2, and L-CDR3 as shown in SEQ ID NO. 37;
H-CDR1, H-CDR2 and H-CDR3 as shown in SEQ ID NO. 14 paired with L-CDR1, L-CDR2, and L-CDR3 as shown in SEQ ID NO. 38;
H-CDR1, H-CDR2 and H-CDR3 as shown in SEQ ID NO. 15 paired with L-CDR1, L-CDR2, and L-CDR3 as shown in SEQ ID NO. 39;
H-CDR1, H-CDR2 and H-CDR3 as shown in SEQ ID NO. 16 paired with L-CDR1, L-CDR2, and L-CDR3 as shown in SEQ ID NO. 40;
H-CDR1, H-CDR2 and H-CDR3 as shown in SEQ ID NO. 17 paired with L-CDR1, L-CDR2, and L-CDR3 as shown in SEQ ID NO. 41;
H-CDR1, H-CDR2 and H-CDR3 as shown in SEQ ID NO. 18 paired with L-CDR1, L-CDR2, and L-CDR3 as shown in SEQ ID NO. 42;
H-CDR1, H-CDR2 and H-CDR3 as shown in SEQ ID NO. 19 paired with L-CDR1, L-CDR2, and L-CDR3 as shown in SEQ ID NO. 43;
H-CDR1, H-CDR2 and H-CDR3 as shown in SEQ ID NO. 20 paired with L-CDR1, L-CDR2, and L-CDR3 as shown in SEQ ID NO. 44;
H-CDR1, H-CDR2 and H-CDR3 as shown in SEQ ID NO. 21 paired with L-CDR1, L-CDR2, and L-CDR3 as shown in SEQ ID NO. 45;
H-CDR1, H-CDR2 and H-CDR3 as shown in SEQ ID NO. 22 paired with L-CDR1, L-CDR2, and L-CDR3 as shown in SEQ ID NO. 46;
H-CDR1, H-CDR2 and H-CDR3 as shown in SEQ ID NO. 23 paired with L-CDR1, L-CDR2, and L-CDR3 as shown in SEQ ID NO. 47; or
H-CDR1, H-CDR2 and H-CDR3 as shown in SEQ ID NO. 24 paired with L-CDR1, L-CDR2, and L-CDR3 as shown in SEQ ID NO. 48.

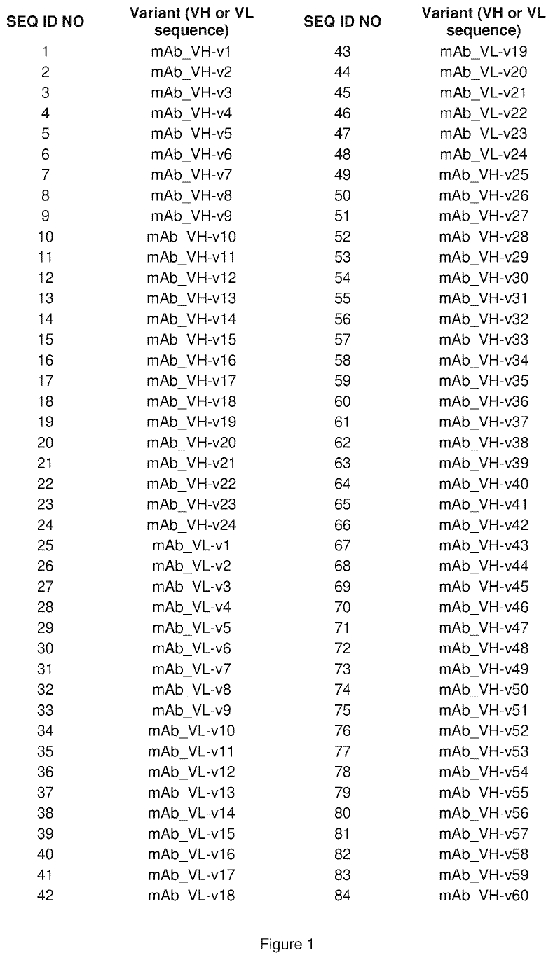

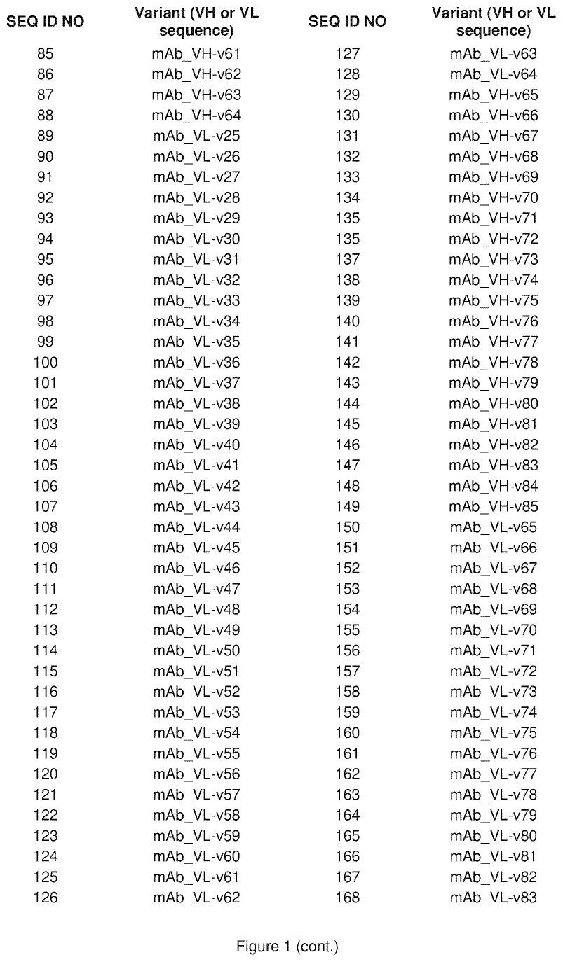

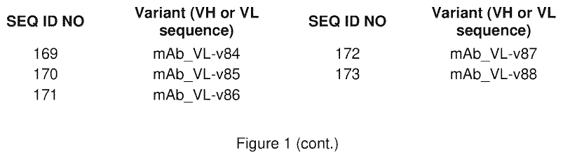

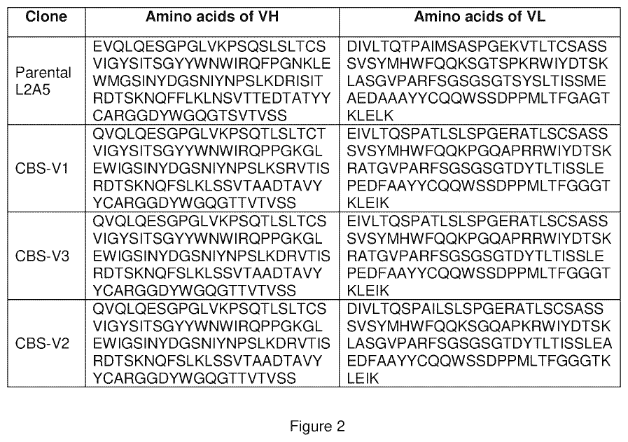

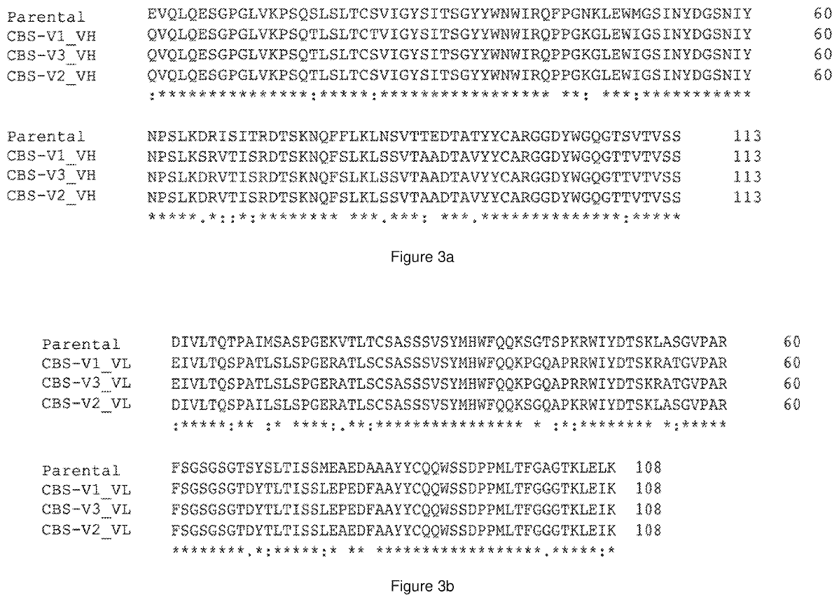

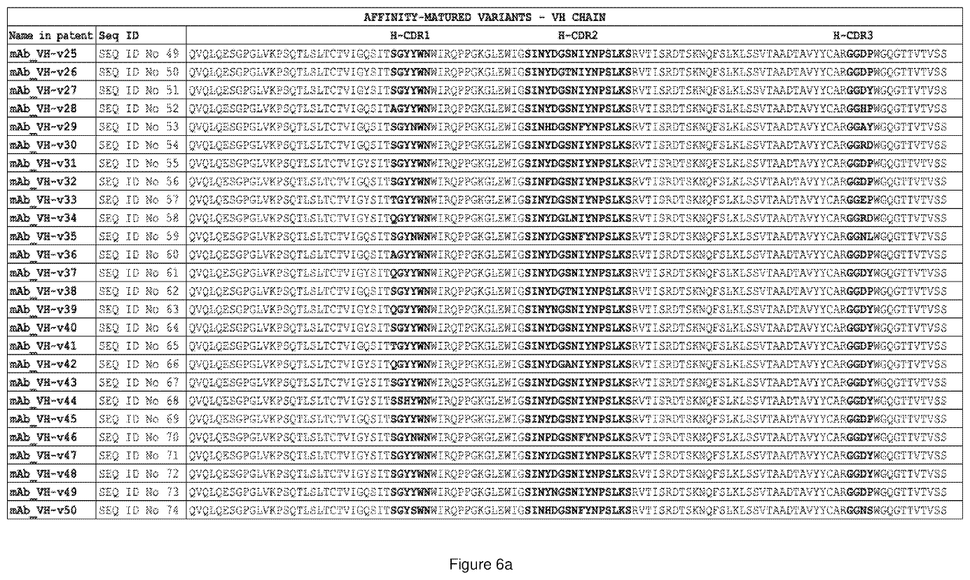

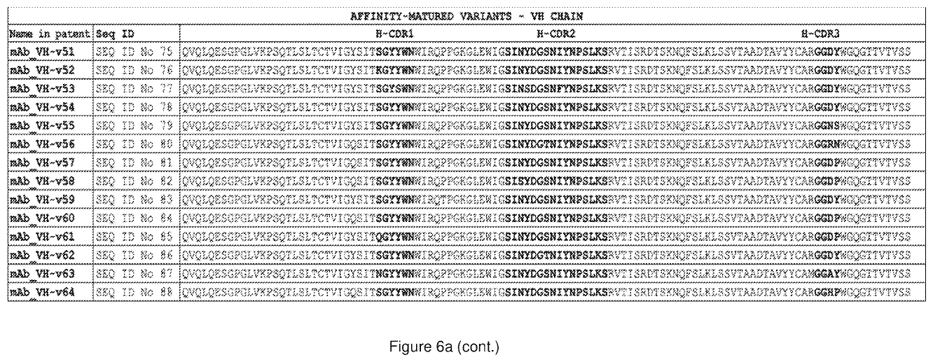

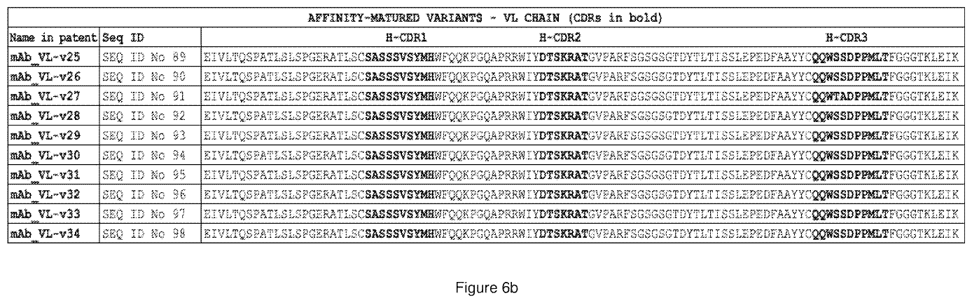

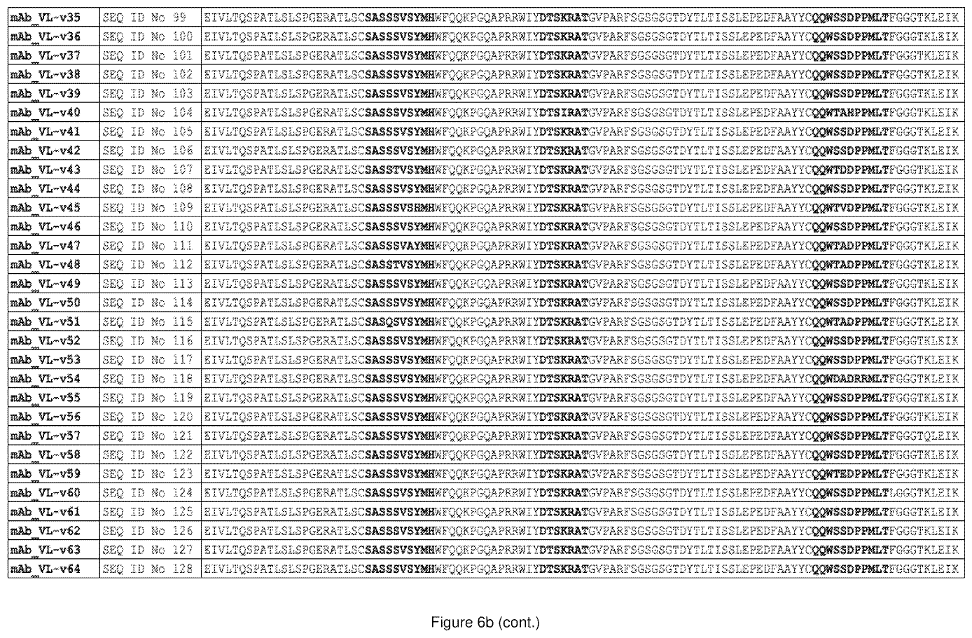

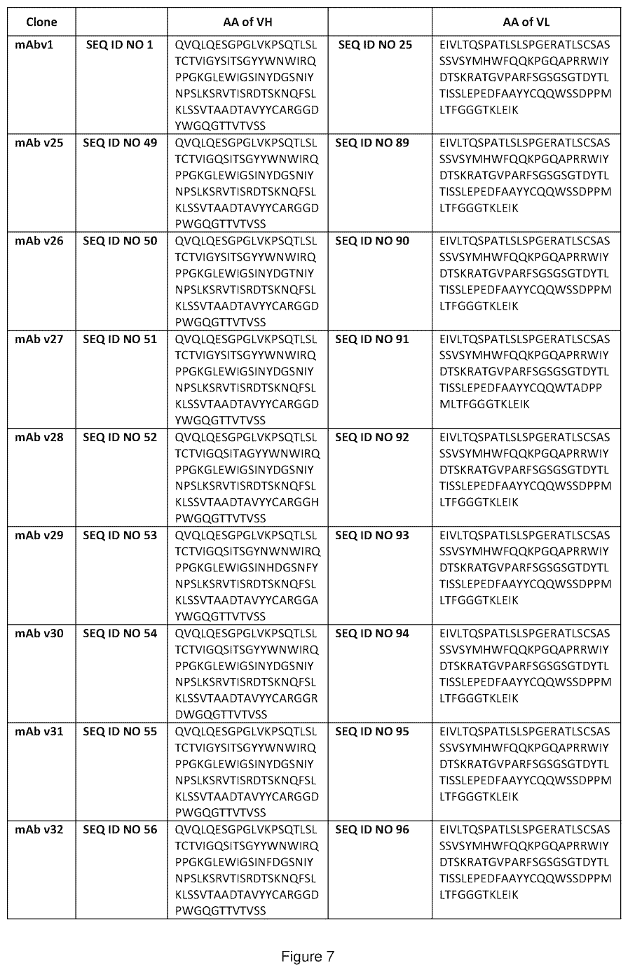

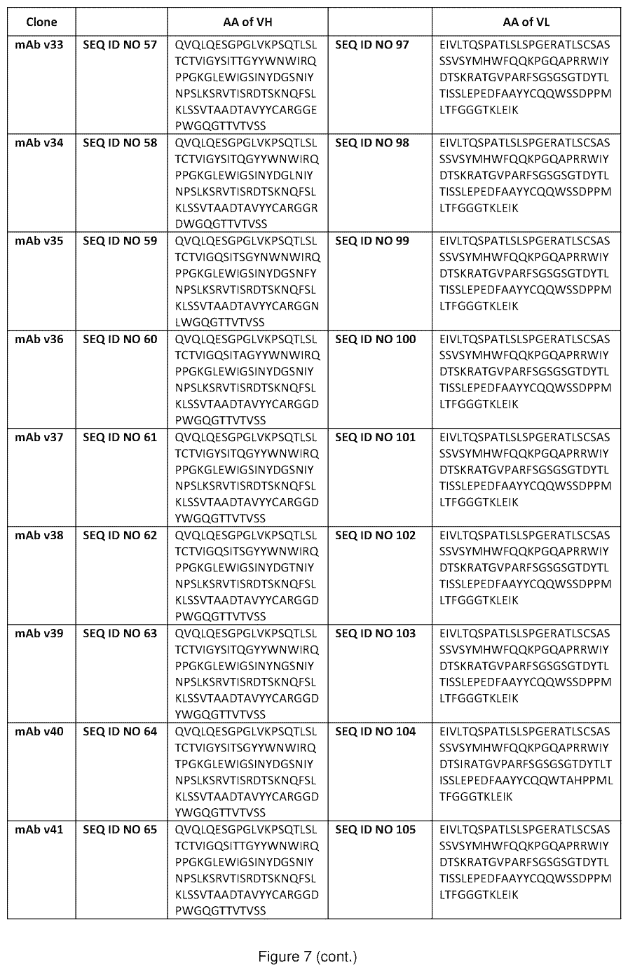

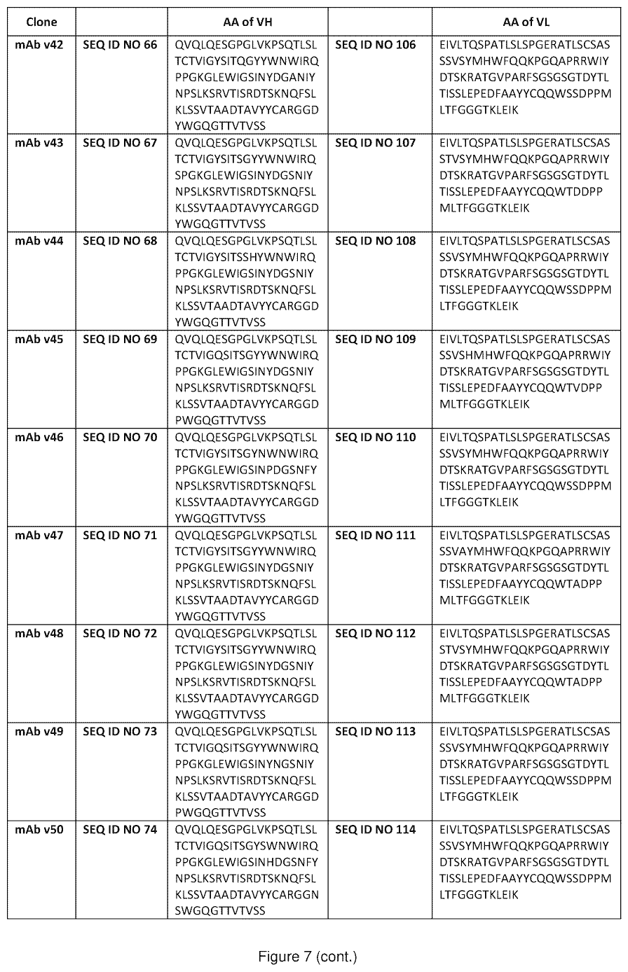

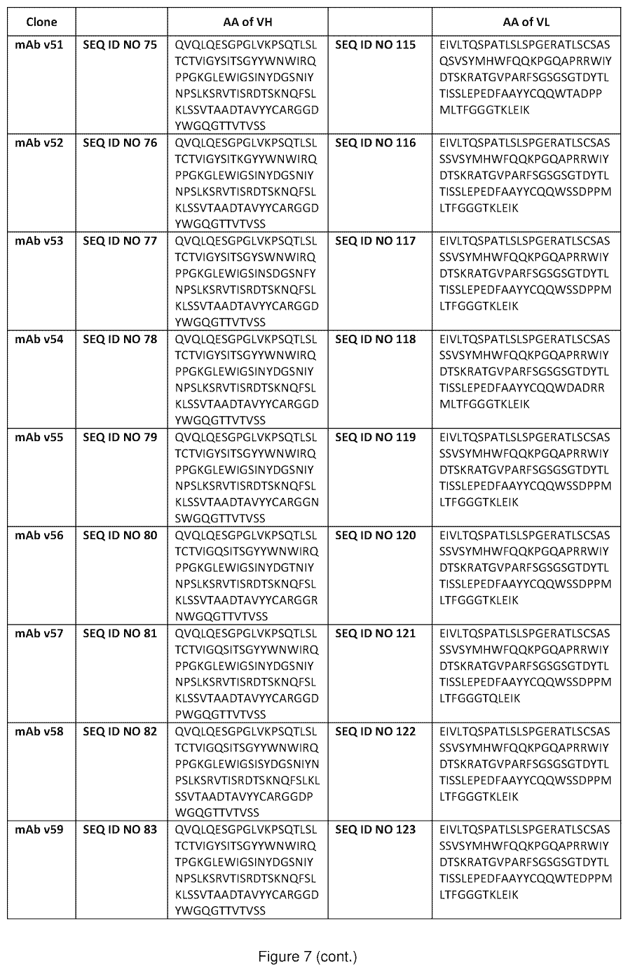

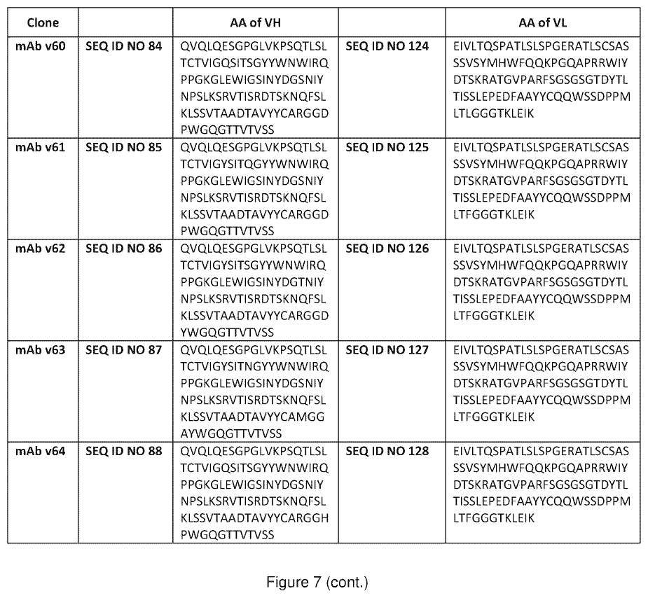

Sequence identity information identifying the humanised and affinity-matured variants provided by the present invention, including the full VH and VL sequences and the CDR regions for each variant is given in FIGS. 1 to 6, and in the sequence listing accompanying this application.

The present invention provides humanised and affinity-matured antibody variants which show excellent binding affinity and specificity to the antigen STn, whilst also showing decreased immunogenicity. Humanised variants are disclosed herein by way of SEQ ID Nos 1-24 (variable VH region) and SEQ ID Nos 25-48 (variable VL region), and affinity-matured antibody variants are disclosed herein by way of SEQ ID Nos 49-88 (variable VH region) and SEQ ID Nos 89-128 (variable VL region),

In one preferred aspect, an antibody or functional antibody fragment or probe thereof is provided, wherein the antibody or fragment or probe comprises one of the following pairs of heavy chain CDRs and light chain CDRs:
H-CDR1, H-CDR2 and H-CDR3 as shown in SEQ ID NO. 17 paired with L-CDR1, L-CDR2, and L-CDR3 as shown in SEQ ID NO. 41;
H-CDR1, H-CDR2 and H-CDR3 as shown in SEQ ID NO. 20 paired with L-CDR1, L-CDR2, and L-CDR3 as shown in SEQ ID NO. 44;
H-CDR1, H-CDR2 and H-CDR3 as shown in SEQ ID NO. 21 paired with L-CDR1, L-CDR2, and L-CDR3 as shown in SEQ ID NO. 45.

In a further aspect, an antibody or functional fragment or probe thereof, is provided, wherein the antibody or fragment or probe comprises one of the following pairs of light chain CDRs and heavy chain CDRs:
H-CDR1, H-CDR2 and H-CDR3 as shown in SEQ ID NO. 49 paired with L-CDR1, L-CDR2, and L-CDR3 as shown in SEQ ID NO. 89;
H-CDR1, H-CDR2 and H-CDR3 as shown in SEQ ID NO. 50 paired with L-CDR1, L-CDR2, and L-CDR3 as shown in SEQ ID NO. 90;
H-CDR1, H-CDR2 and H-CDR3 as shown in SEQ ID NO. 51 paired with L-CDR1, L-CDR2, and L-CDR3 as shown in SEQ ID NO. 91;
H-CDR1, H-CDR2 and H-CDR3 as shown in SEQ ID NO. 52 paired with L-CDR1, L-CDR2, and L-CDR3 as shown in SEQ ID NO. 92;
H-CDR1, H-CDR2 and H-CDR3 as shown in SEQ ID NO. 53 paired with L-CDR1, L-CDR2, and L-CDR3 as shown in SEQ ID NO. 93;
H-CDR1, H-CDR2 and H-CDR3 as shown in SEQ ID NO. 54 paired with L-CDR1, L-CDR2, and L-CDR3 as shown in SEQ ID NO. 94;
H-CDR1, H-CDR2 and H-CDR3 as shown in SEQ ID NO. 55 paired with L-CDR1, L-CDR2, and L-CDR3 as shown in SEQ ID NO. 95;
H-CDR1, H-CDR2 and H-CDR3 as shown in SEQ ID NO. 56 paired with L-CDR1, L-CDR2, and L-CDR3 as shown in SEQ ID NO. 96;
H-CDR1, H-CDR2 and H-CDR3 as shown in SEQ ID NO. 57 paired with L-CDR1, L-CDR2, and L-CDR3 as shown in SEQ ID NO. 97;
H-CDR1, H-CDR2 and H-CDR3 as shown in SEQ ID NO. 58 paired with L-CDR1, L-CDR2, and L-CDR3 as shown in SEQ ID NO. 98;
H-CDR1, H-CDR2 and H-CDR3 as shown in SEQ ID NO. 59 paired with L-CDR1, L-CDR2, and L-CDR3 as shown in SEQ ID NO. 99;
H-CDR1, H-CDR2 and H-CDR3 as shown in SEQ ID NO. 60 paired with L-CDR1, L-CDR2, and L-CDR3 as shown in SEQ ID NO. 100;
H-CDR1, H-CDR2 and H-CDR3 as shown in SEQ ID NO. 61 paired with L-CDR1, L-CDR2, and L-CDR3 as shown in SEQ ID NO. 101;
H-CDR1, H-CDR2 and H-CDR3 as shown in SEQ ID NO. 62 paired with L-CDR1, L-CDR2, and L-CDR3 as shown in SEQ ID NO. 102;
H-CDR1, H-CDR2 and H-CDR3 as shown in SEQ ID NO. 63 paired with L-CDR1, L-CDR2, and L-CDR3 as shown in SEQ ID NO. 103;
H-CDR1, H-CDR2 and H-CDR3 as shown in SEQ ID NO. 64 paired with L-CDR1, L-CDR2, and L-CDR3 as shown in SEQ ID NO. 104;
H-CDR1, H-CDR2 and H-CDR3 as shown in SEQ ID NO. 65 paired with L-CDR1, L-CDR2, and L-CDR3 as shown in SEQ ID NO. 105;
H-CDR1, H-CDR2 and H-CDR3 as shown in SEQ ID NO. 66 paired with L-CDR1, L-CDR2, and L-CDR3 as shown in SEQ ID NO. 106;
H-CDR1, H-CDR2 and H-CDR3 as shown in SEQ ID NO. 67 paired with L-CDR1, L-CDR2, and L-CDR3 as shown in SEQ ID NO. 107;
H-CDR1, H-CDR2 and H-CDR3 as shown in SEQ ID NO. 68 paired with L-CDR1, L-CDR2, and L-CDR3 as shown in SEQ ID NO. 108;
H-CDR1, H-CDR2 and H-CDR3 as shown in SEQ ID NO. 69 paired with L-CDR1, L-CDR2, and L-CDR3 as shown in SEQ ID NO. 109;
H-CDR1, H-CDR2 and H-CDR3 as shown in SEQ ID NO. 70 paired with L-CDR1, L-CDR2, and L-CDR3 as shown in SEQ ID NO. 110;
H-CDR1, H-CDR2 and H-CDR3 as shown in SEQ ID NO. 71 paired with L-CDR1, L-CDR2, and L-CDR3 as shown in SEQ ID NO. 111;
H-CDR1, H-CDR2 and H-CDR3 as shown in SEQ ID NO. 72 paired with L-CDR1, L-CDR2, and L-CDR3 as shown in SEQ ID NO. 112;
H-CDR1, H-CDR2 and H-CDR3 as shown in SEQ ID NO. 73 paired with L-CDR1, L-CDR2, and L-CDR3 as shown in SEQ ID NO. 113;
H-CDR1, H-CDR2 and H-CDR3 as shown in SEQ ID NO. 74 paired with L-CDR1, L-CDR2, and L-CDR3 as shown in SEQ ID NO. 114;
H-CDR1, H-CDR2 and H-CDR3 as shown in SEQ ID NO. 75 paired with L-CDR1, L-CDR2, and L-CDR3 as shown in SEQ ID NO. 115;
H-CDR1, H-CDR2 and H-CDR3 as shown in SEQ ID NO. 76 paired with L-CDR1, L-CDR2, and L-CDR3 as shown in SEQ ID NO. 116;
H-CDR1, H-CDR2 and H-CDR3 as shown in SEQ ID NO. 77 paired with L-CDR1, L-CDR2, and L-CDR3 as shown in SEQ ID NO. 117;
H-CDR1, H-CDR2 and H-CDR3 as shown in SEQ ID NO. 78 paired with L-CDR1, L-CDR2, and L-CDR3 as shown in SEQ ID NO. 118;
H-CDR1, H-CDR2 and H-CDR3 as shown in SEQ ID NO. 79 paired with L-CDR1, L-CDR2, and L-CDR3 as shown in SEQ ID NO. 119;
H-CDR1, H-CDR2 and H-CDR3 as shown in SEQ ID NO. 80 paired with L-CDR1, L-CDR2, and L-CDR3 as shown in SEQ ID NO. 120;
H-CDR1, H-CDR2 and H-CDR3 as shown in SEQ ID NO. 81 paired with L-CDR1, L-CDR2, and L-CDR3 as shown in SEQ ID NO. 121;
H-CDR1, H-CDR2 and H-CDR3 as shown in SEQ ID NO. 82 paired with L-CDR1, L-CDR2, and L-CDR3 as shown in SEQ ID NO. 122;
H-CDR1, H-CDR2 and H-CDR3 as shown in SEQ ID NO. 83 paired with L-CDR1, L-CDR2, and L-CDR3 as shown in SEQ ID NO. 123;
H-CDR1, H-CDR2 and H-CDR3 as shown in SEQ ID NO. 84 paired with L-CDR1, L-CDR2, and L-CDR3 as shown in SEQ ID NO. 124;
H-CDR1, H-CDR2 and H-CDR3 as shown in SEQ ID NO. 85 paired with L-CDR1, L-CDR2, and L-CDR3 as shown in SEQ ID NO. 125;
H-CDR1, H-CDR2 and H-CDR3 as shown in SEQ ID NO. 86 paired with L-CDR1, L-CDR2, and L-CDR3 as shown in SEQ ID NO. 126;
H-CDR1, H-CDR2 and H-CDR3 as shown in SEQ ID NO. 87 paired with L-CDR1, L-CDR2, and L-CDR3 as shown in SEQ ID NO. 127; or
H-CDR1, H-CDR2 and H-CDR3 as shown in SEQ ID NO. 88 paired with L-CDR1, L-CDR2, and L-CDR3 as shown in SEQ ID NO. 128.

In a preferred aspect, an antibody or fragment thereof or probe is provided, wherein the antibody or fragment or probe comprises one of the following pairs of heavy chain CDRs and light chain CDRs:
H-CDR1, H-CDR2 and H-CDR3 as shown in SEQ ID NO. 49 paired with L-CDR1, L-CDR2, and L-CDR3 as shown in SEQ ID NO. 89;
H-CDR1, H-CDR2 and H-CDR3 as shown in SEQ ID NO. 70 paired with L-CDR1, L-CDR2, and L-CDR3 as shown in SEQ ID NO. 110;
H-CDR1, H-CDR2 and H-CDR3 as shown in SEQ ID NO. 81 paired with L-CDR1, L-CDR2, and L-CDR3 as shown in SEQ ID NO. 121; or
H-CDR1, H-CDR2 and H-CDR3 as shown in SEQ ID NO. 88 paired with L-CDR1, L-CDR2, and L-CDR3 as shown in SEQ ID NO. 128.

In a further aspect of the invention, an antibody or functional antibody fragment or probe thereof is provided, wherein the antibody or fragment or probe comprises in addition to the CDR regions described herein:
(a) a heavy chain variable region (VH) wherein the VH comprises a humanised heavy chain framework region
and/or,
(b) a light chain variable region (VL) wherein the VL comprises a humanised light chain framework region.

The humanised framework regions may, for example, be as further described below. It will be understood that the variable framework regions refer to the sequences surrounding the CDR regions. Thus different combinations of the CDR regions and variable framework regions described herein may be made if desired.

Thus, in one aspect of the present invention, an antibody or fragment thereof is provided, wherein the antibody or fragment comprises:
(a) a heavy chain variable region (VH) wherein the VH comprises a humanised heavy chain framework region as shown in the heavy chain variable region (VH) sequences selected from the group consisting of:
(i) any one SEQ ID NOs. 1 to 24 or 49 to 88 respectively
and/or,
(b) a light chain variable region (VL) wherein the VL comprises a humanised light chain framework region as shown in the light chain variable region (VL) sequences selected from the group consisting of:
(ii) any one of SEQ ID NOs. 25 to 48 or 89 to 128 respectively.

One of the above described humanised heavy chain framework regions may be paired with any one of the humanised light chain framework regions, as desired.

Certain pairings of the framework regions are preferred. In one aspect of the invention, there is provided an antibody or fragment thereof wherein the antibody or fragment comprises one of the following pairs of light chain and heavy chain framework regions, wherein the heavy chain framework region is as shown in the heavy chain variable region (VH) sequences shown below, and wherein the light chain framework region is as shown in the light chain variable region (VL) sequences shown below:
heavy chain framework region of SEQ ID NO. 1 paired with light chain framework region from SEQ ID NO. 25;
heavy chain framework region of SEQ ID NO. 2 paired with light chain framework region from SEQ ID NO. 26;
heavy chain framework region of SEQ ID NO. 3 paired with light chain framework region from SEQ ID NO. 27;
heavy chain framework region of SEQ ID NO. 4 paired with light chain framework region from SEQ ID NO. 28;
heavy chain framework region of SEQ ID NO. 5 paired with light chain framework region from SEQ ID NO. 29;
heavy chain framework region of SEQ ID NO. 6 paired with light chain framework region from SEQ ID NO. 30;
heavy chain framework region of SEQ ID NO. 7 paired with light chain framework region from SEQ ID NO. 31;
heavy chain framework region of SEQ ID NO. 8 paired with light chain framework region from SEQ ID NO. 32;
heavy chain framework region of SEQ ID NO. 9 paired with light chain framework region from SEQ ID NO. 33;
heavy chain framework region of SEQ ID NO. 10 paired with light chain framework region from SEQ ID NO. 34;
heavy chain framework region of SEQ ID NO. 11 paired with light chain framework region from SEQ ID NO. 35;
heavy chain framework region of SEQ ID NO. 12 paired with light chain framework region from SEQ ID NO. 36;
heavy chain framework region of SEQ ID NO. 13 paired with light chain framework region from SEQ ID NO. 37;
heavy chain framework region of SEQ ID NO. 14 paired with light chain framework region from SEQ ID NO. 38;
heavy chain framework region of SEQ ID NO. 15 paired with light chain framework region from SEQ ID NO. 39;
heavy chain framework region of SEQ ID NO. 16 paired with light chain framework region from SEQ ID NO. 40;
heavy chain framework region of SEQ ID NO. 17 paired with light chain framework region from SEQ ID NO. 41;
heavy chain framework region of SEQ ID NO. 18 paired with light chain framework region from SEQ ID NO. 42;
heavy chain framework region of SEQ ID NO. 19 paired with light chain framework region from SEQ ID NO. 43;
heavy chain framework region of SEQ ID NO. 20 paired with light chain framework region from SEQ ID NO. 44;
heavy chain framework region of SEQ ID NO. 21 paired with light chain framework region from SEQ ID NO. 45;
heavy chain framework region of SEQ ID NO. 22 paired with light chain framework region from SEQ ID NO. 46;
heavy chain framework region of SEQ ID NO. 23 paired with light chain framework region from SEQ ID NO. 47; or
heavy chain framework region of SEQ ID NO. 24 paired with light chain framework region from SEQ ID NO. 48.

In a further aspect of the invention, there is provided an antibody or fragment thereof or probe thereof wherein the antibody or fragment or probe comprises one of the following pairs of light chain and heavy chain framework regions, wherein the heavy chain framework region is as shown in the heavy chain variable region (VH) sequences shown below, and wherein the light chain framework region is as shown in the light chain variable region (VL) sequences shown below:
heavy chain framework region of SEQ ID NO. 49 paired with light chain framework region from SEQ ID NO. 89;
heavy chain framework region of SEQ ID NO. 50 paired with light chain framework region from SEQ ID NO. 90;
heavy chain framework region of SEQ ID NO. 51 paired with light chain framework region from SEQ ID NO. 91;
heavy chain framework region of SEQ ID NO. 52 paired with light chain framework region from SEQ ID NO. 92;
heavy chain framework region of SEQ ID NO. 53 paired with light chain framework region from SEQ ID NO. 93;
heavy chain framework region of SEQ ID NO. 54 paired with light chain framework region from SEQ ID NO. 94;
heavy chain framework region of SEQ ID NO. 55 paired with light chain framework region from SEQ ID NO. 95;
heavy chain framework region of SEQ ID NO. 56 paired with light chain framework region from SEQ ID NO. 96;
heavy chain framework region of SEQ ID NO. 57 paired with light chain framework region from SEQ ID NO. 97;
heavy chain framework region of SEQ ID NO. 58 paired with light chain framework region from SEQ ID NO. 98;
heavy chain framework region of SEQ ID NO. 59 paired with light chain framework region from SEQ ID NO. 99;
heavy chain framework region of SEQ ID NO. 60 paired with light chain framework region from SEQ ID NO. 100;
heavy chain framework region of SEQ ID NO. 61 paired with light chain framework region from SEQ ID NO. 101;
heavy chain framework region of SEQ ID NO. 62 paired with light chain framework region from SEQ ID NO. 102;
heavy chain framework region of SEQ ID NO. 63 paired with light chain framework region from SEQ ID NO. 103;
heavy chain framework region of SEQ ID NO. 64 paired with light chain framework region from SEQ ID NO. 104;
heavy chain framework region of SEQ ID NO. 65 paired with light chain framework region from SEQ ID NO. 105;
heavy chain framework region of SEQ ID NO. 66 paired with light chain framework region from SEQ ID NO. 106;
heavy chain framework region of SEQ ID NO. 67 paired with light chain framework region from SEQ ID NO. 107;
heavy chain framework region of SEQ ID NO. 68 paired with light chain framework region from SEQ ID NO. 108;
heavy chain framework region of SEQ ID NO. 69 paired with light chain framework region from SEQ ID NO. 109;
heavy chain framework region of SEQ ID NO. 70 paired with light chain framework region from SEQ ID NO. 110;
heavy chain framework region of SEQ ID NO. 71 paired with light chain framework region from SEQ ID NO. 111;
heavy chain framework region of SEQ ID NO. 72 paired with light chain framework region from SEQ ID NO. 112;
heavy chain framework region of SEQ ID NO. 73 paired with light chain framework region from SEQ ID NO. 113;
heavy chain framework region of SEQ ID NO. 74 paired with light chain framework region from SEQ ID NO. 114;
heavy chain framework region of SEQ ID NO. 75 paired with light chain framework region from SEQ ID NO. 115;
heavy chain framework region of SEQ ID NO. 76 paired with light chain framework region from SEQ ID NO. 116;
heavy chain framework region of SEQ ID NO. 77 paired with light chain framework region from SEQ ID NO. 117;
heavy chain framework region of SEQ ID NO. 78 paired with light chain framework region from SEQ ID NO. 118;
heavy chain framework region of SEQ ID NO. 79 paired with light chain framework region from SEQ ID NO. 119;
heavy chain framework region of SEQ ID NO. 80 paired with light chain framework region from SEQ ID NO. 120;
heavy chain framework region of SEQ ID NO. 81 paired with light chain framework region from SEQ ID NO. 121;
heavy chain framework region of SEQ ID NO. 82 paired with light chain framework region from SEQ ID NO. 122;
heavy chain framework region of SEQ ID NO. 83 paired with light chain framework region from SEQ ID NO. 123;
heavy chain framework region of SEQ ID NO. 84 paired with light chain framework region from SEQ ID NO. 124;
heavy chain framework region of SEQ ID NO. 85 paired with light chain framework region from SEQ ID NO. 125;
heavy chain framework region of SEQ ID NO. 86 paired with light chain framework region from SEQ ID NO. 126;
heavy chain framework region of SEQ ID NO. 87 paired with light chain framework region from SEQ ID NO. 127; or heavy chain framework region of SEQ ID NO. 88 paired with light chain framework region from SEQ ID NO. 128.

It is the case that the framework regions allow a certain degree of variability in the exact sequence, whilst still allowing for maintenance of function, including binding affinity and specificity. Accordingly, the invention also provides an antibody or fragment or probe thereof as described, wherein the heavy chain framework region and/or the light chain framework region may have 80% or more, preferably 85% or more, sequence identity to the specific sequences recited herein. For example, this may be by way of substitution, addition, or deletion of amino acid residues, with substitution often being preferred. Substitutions may, for example, be conservative amino acid substitutions.

In a preferred aspect of the invention, the antibody or fragment or probe thereof as described herein is such that the heavy chain framework region and/or the light chain framework region have 90% or more, preferably 95% or more, sequence identity to the specific sequences recited herein. For example, this may be by way of substitution, addition, or deletion of amino acid residues, with substitution often being preferred. Substitutions may, for example, be conservative amino acid substitutions.

In a further aspect of the present invention, there is provided an antibody or fragment thereof or probe thereof wherein the antibody or fragment or probe comprises:
(a) a heavy chain variable region (VH) selected from the group consisting of:
(i) any one SEQ ID NOs. 1 to 24 or 49 to 88 respectively
and/or,
(a) a light chain variable region (VL) selected from the group consisting of:
(ii) any one of SEQ ID NOs. 25 to 48 or 89 to 128 respectively.

Any one of the VH regions may be paired with any one of the VL regions, although certain pairings are preferred.

Thus, in a further aspect, the present invention provides an antibody or fragment thereof or probe thereof as described herein, wherein the antibody or fragment or probe comprises one of the following pairs of heavy chain variable regions (VH) and light chain variable regions (VL):
SEQ ID NO. 1 paired with SEQ ID NO. 25;
SEQ ID NO. 2 paired with SEQ ID NO. 26;
SEQ ID NO. 3 paired with SEQ ID NO. 27;
SEQ ID NO. 4 paired with SEQ ID NO. 28;
SEQ ID NO. 5 paired with SEQ ID NO. 29;
SEQ ID NO. 6 paired with SEQ ID NO. 30;
SEQ ID NO. 7 paired with SEQ ID NO. 31;
SEQ ID NO. 8 paired with SEQ ID NO. 32;
SEQ ID NO. 9 paired with SEQ ID NO. 33;
SEQ ID NO. 10 paired with SEQ ID NO. 34;
SEQ ID NO. 11 paired with SEQ ID NO. 35;
SEQ ID NO. 12 paired with SEQ ID NO. 36;
SEQ ID NO. 13 paired with SEQ ID NO. 37;
SEQ ID NO. 14 paired with SEQ ID NO. 38;
SEQ ID NO. 15 paired with SEQ ID NO. 39;
SEQ ID NO. 16 paired with SEQ ID NO. 40;
SEQ ID NO. 17 paired with SEQ ID NO. 41;
SEQ ID NO. 18 paired with SEQ ID NO. 42;
SEQ ID NO. 19 paired with SEQ ID NO. 43;
SEQ ID NO. 20 paired with SEQ ID NO. 44;
SEQ ID NO. 21 paired with SEQ ID NO. 45;
SEQ ID NO. 22 paired with SEQ ID NO. 46;
SEQ ID NO. 23 paired with SEQ ID NO. 47; or
SEQ ID NO. 24 paired with SEQ ID NO. 48.

In a further aspect of the invention, an antibody or fragment thereof or probe thereof is provided wherein the antibody or fragment or probe comprises one of the following pairs of heavy chain variable regions (VH) and light chain variable regions (VL):
SEQ ID NO. 49 paired with SEQ ID NO. 89;
SEQ ID NO. 50 paired with SEQ ID NO. 90;
SEQ ID NO. 51 paired with SEQ ID NO. 91;
SEQ ID NO. 52 paired with SEQ ID NO. 92;
SEQ ID NO. 53 paired with SEQ ID NO. 93;
SEQ ID NO. 54 paired with SEQ ID NO. 94;
SEQ ID NO. 55 paired with SEQ ID NO. 95;
SEQ ID NO. 56 paired with SEQ ID NO. 96;
SEQ ID NO. 57 paired with SEQ ID NO. 97;
SEQ ID NO. 58 paired with SEQ ID NO. 98;
SEQ ID NO. 59 paired with SEQ ID NO. 99;
SEQ ID NO. 60 paired with SEQ ID NO. 100;
SEQ ID NO. 61 paired with SEQ ID NO. 101;
SEQ ID NO. 62 paired with SEQ ID NO. 102;
SEQ ID NO. 63 paired with SEQ ID NO. 103;
SEQ ID NO. 64 paired with SEQ ID NO. 104;
SEQ ID NO. 65 paired with SEQ ID NO. 105;
SEQ ID NO. 66 paired with SEQ ID NO. 106;
SEQ ID NO. 67 paired with SEQ ID NO. 107;
SEQ ID NO. 68 paired with SEQ ID NO. 108;
SEQ ID NO. 69 paired with SEQ ID NO. 109;
SEQ ID NO. 70 paired with SEQ ID NO. 110;
SEQ ID NO. 71 paired with SEQ ID NO. 111;
SEQ ID NO. 72 paired with SEQ ID NO. 112;
SEQ ID NO. 73 paired with SEQ ID NO. 113;
SEQ ID NO. 74 paired with SEQ ID NO. 114;
SEQ ID NO. 75 paired with SEQ ID NO. 115;
SEQ ID NO. 76 paired with SEQ ID NO. 116;
SEQ ID NO. 77 paired with SEQ ID NO. 117;
SEQ ID NO. 78 paired with SEQ ID NO. 118;
SEQ ID NO. 79 paired with SEQ ID NO. 119;
SEQ ID NO. 80 paired with SEQ ID NO. 120;
SEQ ID NO. 81 paired with SEQ ID NO. 121;
SEQ ID NO. 82 paired with SEQ ID NO. 122;
SEQ ID NO. 83 paired with SEQ ID NO. 123;
SEQ ID NO. 84 paired with SEQ ID NO. 124;
SEQ ID NO. 85 paired with SEQ ID NO. 125;
SEQ ID NO. 86 paired with SEQ ID NO. 126
SEQ ID NO. 87 paired with SEQ ID NO. 127; or
SEQ ID NO. 88 paired with SEQ ID NO. 128.

In the same way as described above, the invention also provides an antibody or fragment thereof or probe thereof as described, wherein the heavy chain variable region (VH) and/or the light chain variable region (VL) may have 80% or more, preferably 85% or more, sequence identity to the specific VH and VL sequences recited herein. For example, this may be by way of substitution, addition, or deletion of amino acid residues, with substitution often being preferred. Substitutions may, for example, be conservative amino acid substitutions.

In a preferred aspect of the invention, the antibody or fragment or probe thereof as described herein is such that the heavy chain variable region (VH) and/or the light chain variable region have 90% or more, preferably 95% or more, sequence identity to the specific sequences recited herein. For example, this may be by way of substitution, addition, or deletion of amino acid residues, with substitution often being preferred. Substitutions may, for example, be conservative amino acid substitutions.

In terms of functionality, the invention provides an antibody or fragment thereof, or a probe thereof, as described herein, that binds to STn and a group of glycans terminated by alpha 2,6-linked sialic acids. The glycans terminated by alpha 2,6-linked sialic acids may comprise for example STn, 2,6-sialyl T, di-sialyl T, or 2,6-sialolactosamine.

An overall group of glycans recognized by the antibody or fragment thereof, or a probe thereof, as described herein includes:

1. Sialyl Tn:
NeuAcα--6GalNAcα/β1-
2. 2,6-sialyl T:
Gal β1-3GalNAca1-NeuAcα2-6
3. di sialyl T:
NeuAcα2-3Galβ1-3GalNAcα1---NeuAcα2-6
4. 2,6-sialo-N-acetyllactosamine:
NeuAcα2-6Galβ1-4Glcβ1-

The sialyl Tn, aka, STn, sialosyl Tn, sialylated Tn, Neu5Ac-α2, 6GalNAcα-O-Ser/Thr, or also referred to as CD175s by the “cluster of differentiation’ nomenclature, is the simplest sialylated mucin-type O-glycan. The STn is a truncated O-glycan containing a sialic acid (Neu5Ac) α-2,6 linked (via carbon 6} to N-acetyl-galactosamine (GalNAc) alpha-O-linked to a serine/threonine (Ser/Thr) (Neu5Ac-α2, 6GalNAcα-O-Ser/Thr). The sialylation prevents the formation of various core structures otherwise found in mucin-type O-glycans.

STn is expressed by more than 80% of human carcinomas and is associated with poor prognosis and decreased overall survival in different cancer patients. The biosynthesis of the STn antigen has been linked to the expression of the sialyltransferase ST6GalNAc1, and to mutations or loss of heterozygosity of the COSMC gene.

Antibodies that bind to STn with such specificity are particularly interesting because of their high tumour specificity and low or absent reactivity to normal cells, in contrast to many current antibody therapies.

An antibody or fragment thereof or probe thereof as described, is able to specifically bind to STn or a group of alpha-2,6 sialylated glycans, as described herein.

The antibodies or fragments thereof described herein, may be subject to glycan changes at glycosylation sites.

In one aspect of the invention, an antibody or fragment thereof or probe thereof as described herein, may be provided in any suitable form. For example, the antibody or fragment or probe may be provided as a ScFv, monoclonal antibody, chimeric antibody, humanized antibody, bispecific antibody, antibody drug conjugate (ADC) or CAR-T-cell, or other format, as will be understood by the skilled person.

Thus, for example, an antibody or fragment or probe thereof of the present invention, may be provided as a single-chain fragment variable antibody (scFv). This refers to a functional antibody fragment containing only the VL and VH regions, which are joined by a linker, forming a monovalent antigen binding site. Diabodies, tribodies and tetrabodies are antibodies including dimers, trimers or tetramers of scFv, i. e. containing two, three and four polypeptide chains respectively, and forming two, three and four antigen binding sites respectively, which can be the same or different.

The antibody, functional antibody fragment or probes thereof, in the present invention, may have one or more binding sites. If containing more than one binding site, these sites can be identical to one another or can be different. In the case of two different binding sites, the antibody, functional antibody fragment or probe thereof, is named a “bispecific” antibody.

In a further aspect, the present invention thus also provides an antibody drug conjugate (ADC) comprising an antibody or fragment or probe thereof, a linker and a cytotoxic drug, as herein described.

The invention also provides a pharmaceutical composition comprising an antibody or functional antibody fragment or probe thereof, or an antibody drug conjugate (ADC) and a pharmaceutically acceptable carrier, as described herein.

In another aspect, there is provided a method of detecting a tumour biomarker in a patient sample using an antibody or functional antibody fragment or probe thereof, or an antibody drug conjugate (ADC), or a pharmaceutical composition as described herein. The methodology involves the staining of biological samples obtained from a subject with the nucleotide sequences encoding an antibody or functional antibody fragment or probe thereof, or an antibody drug conjugate (ADC), as described herein, under suitable conditions for specific binding to the said antibody. The presence or absence of binding of the said antibody is indicative of tumour cells expressing cell surface STn, 2,6-sialyl T, di-sialyl T, or 2,6-sialolactosamine. For example, the biological samples analysed, may include isolated cells, or tissue, or tumour derived proteins.

The invention also provides an antibody or functional antibody fragment or probe thereof, or an antibody drug conjugate (ADC), or a pharmaceutical composition for medical use, all of them as herein described. In particular, the antibody as a pharmaceutical composition for medical use aims at being used for treating cancer patients. It is envisaged that various types of tumours may be treated with the variants disclosed herein.

Thus, in some aspects, the antibody, functional antibody fragment or probe thereof of the invention, may be conjugated or fused to one or more diagnostic or therapeutic agents, or any other desired molecules. The resulting conjugated antibody, functional antibody fragment or probe thereof, can be useful to monitor or diagnose the onset development, progression and/or severity of a disease associated with the expression of STn or alpha-2, 6 sialylated glycans.

An antibody or functional fragment or probe thereof of the invention may also be used to detect the expression of STn or alpha-2,6 sialylated glycans in any biological sample using classical immunohistological methods (IHC or immunoassays, such as the enzyme linked immunosorbent assay (ELISA) and the radioimmunoassay (RIA), flow cytometry, and immunoblotting.

The antibody, functional antibody fragment or probe thereof of the invention may be included alone, conjugated, or in combination with a pharmaceutical composition, provided in an effective concentration, to wield a therapeutically useful effect, with minimal side effects.

In a further aspect, the invention includes an isolated polynucleotide comprising a nucleic acid sequence, wherein the nucleic acid sequence encodes an antibody or functional antibody fragment or probe thereof, as described herein, in particular the variable heavy chain region of the antibody, the variable light chain region domain of the antibody, or functional antibody fragments or probes thereof.

In a further aspect, the present invention provides an expression vector comprising a polynucleotide encoding an antibody or fragment or probe as described herein. As will be understood, a suitable host cell comprising such an expression vector may be provided.

The present invention further provides a pharmaceutical composition comprising a nucleotide sequence encoding a monoclonal antibody or a functional antibody fragment thereof, preferably an antibody or fragment as described and claimed herein, wherein said nucleotide sequence is encapsulated using an mRNA delivery system. Such systems are known in the art. The mRNA delivery system enables transient expression of the antibody or antibody fragment in a target cell, facilitating the production of the therapeutic protein within the cell.

The mRNA delivery system comprising a nucleotide sequence encoding a monoclonal antibody or a functional antibody fragment, preferably an antibody or fragment as described and claimed herein, includes various approaches, including lipid-based nanoparticles (LNPs), polymer-based nanoparticles, and viral vectors, allowing efficient delivery to target cells.

For example, in the case of LNPs, the formulation typically consists of a lipid-based nanoparticle composed of lipids, such as phospholipids and cholesterol, along with a nucleotide sequence encoding the antibody or antibody fragment. The formulation allows for the encapsulation of the nucleotide sequence within the lipid bilayer structure, protecting it from degradation and facilitating its delivery to the target cells.

In one embodiment, LNPs can be prepared by combining the lipids and the nucleotide sequence in an appropriate solvent, followed by a controlled mixing process, such as microfluidics or sonication, to form small and stable nanoparticles. The resulting LNPs can be further optimized by adjusting the lipid composition, nucleotide sequence concentration, and other formulation parameters to achieve desirable characteristics, such as size, stability, and cellular uptake efficiency.

To achieve efficient delivery, LNPs can be administered to the subject via various routes, including intravenous, intramuscular, or subcutaneous injection. Upon administration, LNPs enter the target cells through endocytosis, and the encapsulated nucleotide sequence is released into the cytoplasm. The nucleotide sequence is then translated into the desired antibody or antibody fragment, leading to transient expression within the target cells.

The use of LNPs as an mRNA delivery system allows for controlled and transient production of the therapeutic antibody or antibody fragment in a targeted manner. This technology offers advantages such as ease of formulation, high encapsulation efficiency, and calibrated delivery properties, making it an effective approach for delivering nucleotide sequences encoding antibodies or antibody fragments for therapeutic applications.

The invention thus also provides a composition comprising a nucleotide sequence encoding a monoclonal antibody or a functional antibody fragment thereof, wherein the antibody or fragment is as described and claimed herein, wherein said nucleotide sequence is encapsulated within an mRNA delivery system, wherein said mRNA delivery system is capable of facilitating transient expression of the sTn antibody or antibody fragment in a target cell.

The mRNA delivery system may be any suitable system, but in a preferred aspect the mRNA delivery system comprises lipid-based nanoparticles, polymer-based nanoparticles, or viral vectors. Lipid-based nanoparticles (LNPs) are particularly suitable, as noted above.

In a further aspect, there is provided a method for delivering a monoclonal antibody or a functional antibody fragment thereof to a target cell, preferably an antibody or fragment as described and claimed herein, which method comprises: a) encapsulating a nucleotide sequence encoding said antibody or antibody fragment within a mRNA delivery system; b) administering said mRNA delivery system to the target cell; c) allowing the mRNA to be taken up by the target cell and expressed, thereby producing the antibody or antibody fragment within the target cell.

In one aspect, the mRNA delivery system may be further modified with targeting moieties to enhance specific uptake by target cells expressing the sialyl Tn (STn) antigen.

In a further aspect, there is also provided a therapeutic composition comprising an mRNA delivery system as described herein, and a nucleotide sequence encoding a monoclonal antibody or a functional antibody fragment thereof specifically binding to the sialyl Tn (STn) antigen, preferably an antibody or fragment as described and claimed herein, wherein said composition is formulated for administration to a human or animal subject. The therapeutic composition may for example further comprise a carrier or excipient suitable for systemic or localized administration to the subject, as will be understood.

In a further aspect, the invention provides a method for treating cancer characterized by the overexpression of the sialyl Tn (STn) antigen, which method comprises: a) incorporating a nucleotide sequence encoding a monoclonal antibody or a functional antibody fragment thereof binding to the STn antigen, in an mRNA delivery system; b) administering said mRNA delivery system to a subject in need thereof, thereby allowing expression of the antibody or antibody fragment within the subject's cells and selectively targeting STn-expressing cancer cells. The monoclonal antibody or a functional antibody fragment thereof binding to the STn antigen is preferably an antibody or fragment as described and claimed herein.

The invention also provides a theranostic composition comprising an mRNA delivery system which includes a nucleotide sequence encoding a monoclonal antibody or a functional antibody fragment thereof, preferably an antibody or fragment as described and claimed herein, specifically binding to the sialyl Tn (STn) antigen, and a detectable label, wherein said composition allows for both therapeutic delivery to, and imaging of, STn-expressing cancer cells.

In a further aspect, there is also provided a method for treating cancer in animals or humans characterized by overexpression of the sialyl Tn (STn) antigen, comprising administering to a subject in need thereof a therapeutic composition comprising an antibody or fragment thereof, preferably an antibody or fragment as described and claimed herein, thereby selectively targeting and treating STn-expressing cancer cells in animals or humans.

In a further aspect, the invention also provides a cell therapy composition comprising an sTn antibody, preferably an antibody or fragment thereof as described and claimed herein, for engaging and directing an Effector Cell to a target, wherein said Cell Therapy comprises technologies such as Chimeric Antigen Receptor (CAR) therapy, Natural Killer (NK) cell therapy, and cell-specific engagers, thereby facilitating targeted recognition and cytotoxicity against sTn-expressing cells.

The invention also provides the use of an sTn antibody, preferably an antibody or fragment thereof as described and claimed herein, in a Cell Therapy application, wherein said sTn Antibody sequences are incorporated into Effector Cells through genetic modification, enabling specific recognition and enhanced cytotoxicity against target cells expressing sTn antigen, in conjunction with various technologies including CAR-T, NK CAR, and cell-specific engagers.

A further aspect of the invention provides the use of an sTn antibody, preferably an antibody or fragment thereof as described and claimed herein, in a Cell Therapy application for directing Effector Cells to sTn-expressing target cells, wherein said Cell Therapy comprises a range of technologies comprising, but not limited to, CAR-T, NK CAR, and cell-specific engagers, thereby promoting targeted cytotoxicity and immune-mediated responses against sTn-expressing cells.

In a further aspect, there is also provided the use of an sTn antibody, preferably an antibody or fragment thereof as described and claimed herein, in a Cell Therapy application, wherein the sTn Antibody is employed to engage and direct Effector Cells through various technologies, including CAR-T, NK CAR, and cell-specific engagers, enabling targeted recognition, activation, and cytotoxicity against cells expressing the sTn antigen.

In a yet further aspect, there is provided the use of an sTn antibody, preferably an antibody or fragment thereof as described and claimed herein, in a Cell Therapy application for enabling targeted recognition and cytotoxicity by Effector Cells, wherein said Cell Therapy comprises a range of technologies such as CAR-T, NK CAR, and cell-specific engagers, facilitating the engagement of Effector Cells to sTn-expressing cells and promoting immune-mediated responses.

In accordance with the invention, a method of producing an antibody or functional antibody fragment or probe thereof, as described herein may comprise using such a suitable host cell.

The present invention also provides mRNA sequences that encode antibodies targeting the sTn antigen, such as the antibodies described and claimed herein. The invention takes advantage of the degeneracy of the genetic code, which allows for the design of multiple mRNA sequences encoding the same antibody but optimized for expression in human cells.

For example, the invention provides mRNA sequences encoding the variable heavy (VH) and light (VL) chains of an anti-sTn antibody, and some specific examples are given below. Using the standard genetic code and considering factors such as codon usage bias, mRNA stability, and immunogenicity, it is possible to generate an optimized mRNA sequence for each chain.

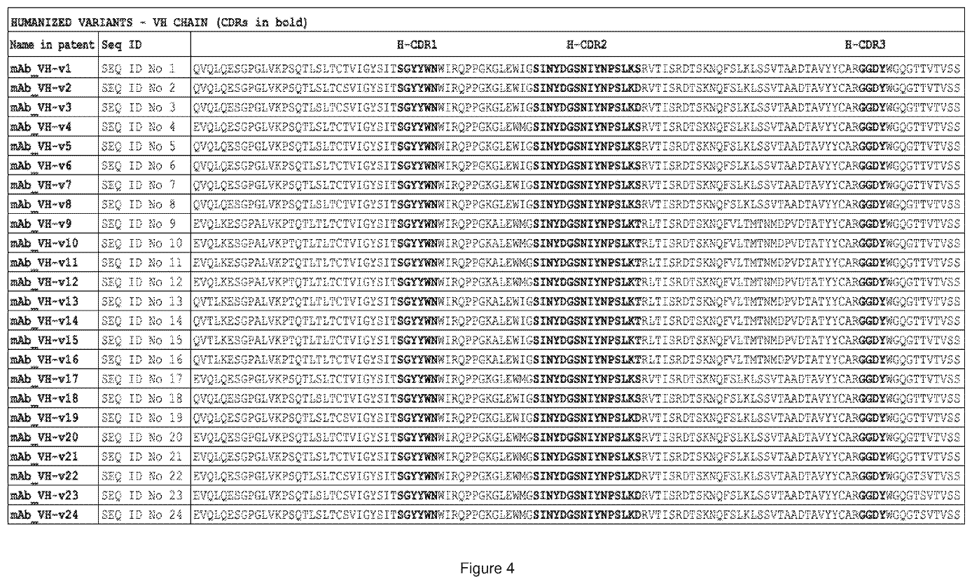

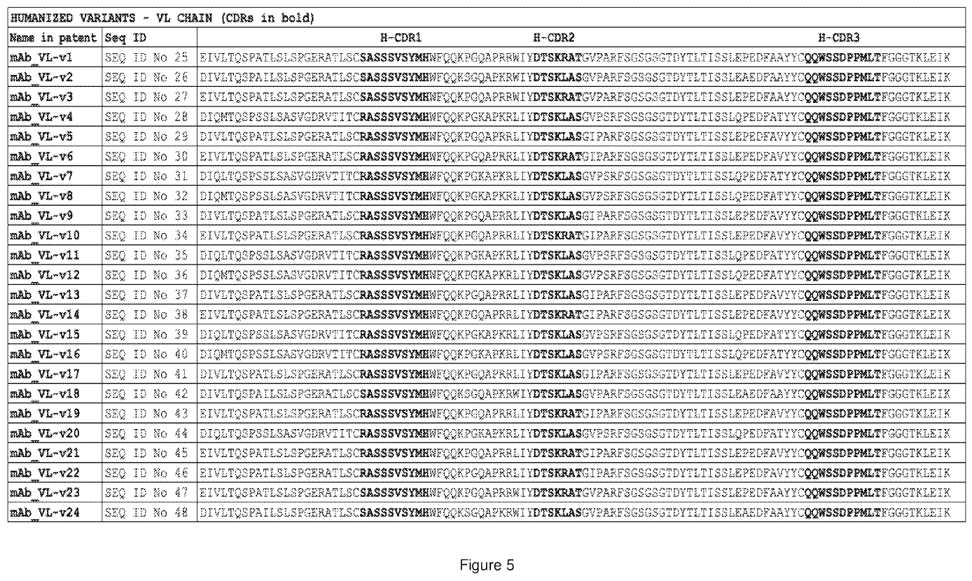

As an illustrative embodiment of the invention, the amino acid sequences for the VH and VL chains of an anti-sTn antibody relative to the following mAb clones are, for example, given in FIGS. 4 to 6:

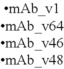

The corresponding optimized nucleotide sequences, generated using the standard genetic code and considering factors such as codon usage bias in humans, mRNA stability, and immunogenicity, are as follows:

VH sequences:
For mAb_v1 and mAb_v48 (and designated nucleotide SEQ ID No: 174):
CAGGUGCAGCUGCAGGAGAGCGGCCCCGGCCUGGUGAAGCCCAGCCAGACCCUGAGCC
UGACCUGCACCGUGAUCGGCUACAGCAUCACCAGCGGCUACUACUGGAACUGGAUCAG
GCAGCCCCCCGGCAAGGGCCUGGAGUGGAUCGGCAGCAUCAACUACGACGGCAGCAAC
AUCUACAACCCCAGCCUGAAGAGCAGGGUGACCAUCAGCAGGGACACCAGCAAGAACCA
GUUCAGCCUGAAGCUGAGCAGCGUGACCGCCGCCGACACCGCCGUGUACUACUGCGCC
AGGGGCGGCGACUACUGGGGCCAGGGCACCACCGUGACCGUGAGCAGC
For mAb_v64 (and designated nucleotide SEQ ID No: 175):
CAGGUGCAGCUGCAGGAGAGCGGCCCCGGCCUGGUGAAGCCCAGCCAGACCCUGAGCC
UGACCUGCACCGUGAUCGGCCAGAGCAUCACCAGCGGCUACUACUGGAACUGGAUCAG
GCAGCCCCCCGGCAAGGGCCUGGAGUGGAUCGGCAGCAUCAACUACGACGGCAGCAAC
AUCUACAACCCCAGCCUGAAGAGCAGGGUGACCAUCAGCAGGGACACCAGCAAGAACCA
GUUCAGCCUGAAGCUGAGCAGCGUGACCGCCGCCGACACCGCCGUGUACUACUGCGCC
AGGGGCGGCCACCCCUGGGGCCAGGGCACCACCGUGACCGUGAGCAGC
For mAb_v46 (and designated nucleotide SEQ ID No: 176):
CAGGUGCAGCUGCAGGAGAGCGGCCCCGGCCUGGUGAAGCCCAGCCAGACCCUGAGCC
UGACCUGCACCGUGAUCGGCUACAGCAUCACCAGCGGCUACAACUGGAACUGGAUCAG
GCAGCCCCCCGGCAAGGGCCUGGAGUGGAUCGGCAGCAUCAACCCCGACGGCAGCAAC
UUCUACAACCCCAGCCUGAAGAGCAGGGUGACCAUCAGCAGGGACACCAGCAAGAACCA
GUUCAGCCUGAAGCUGAGCAGCGUGACCGCCGCCGACACCGCCGUGUACUACUGCGCC
AGGGGCGGCGACUACUGGGGCCAGGGCACCACCGUGACCGUGAGCAGC
VL sequences:
For mAb_v1 and mAb_v64; mAb_v46 (and designated nucleotide SEQ ID No: 177):
GAGAUCGUGCUGACCCAGAGCCCCGCCACCCUGAGCCUGAGCCCCGGCGAGAGGGCCA
CCCUGAGCUGCAGCGCCAGCAGCAGCGUGAGCUACAUGCACUGGUUCCAGCAGAAGCC
CGGCCAGGCCCCCAGGAGGUGGAUCUACGACACCAGCAAGAGGGCCACCGGCGUGCCC
GCCAGGUUCAGCGGCAGCGGCAGCGGCACCGACUACACCCUGACCAUCAGCAGCCUGG
AGCCCGAGGACUUCGCCGCCUACUACUGCCAGCAGUGGAGCAGCGACCCCCCCAUGCU
GACCUUCGGCGGCGGCACCAAGCUGGAGAUCAAG
For mAb_v48 (and designated nucleotide SEQ ID No: 178):
GAGAUCGUGCUGACCCAGAGCCCCGCCACCCUGAGCCUGAGCCCCGGCGAGAGGGCCA
CCCUGAGCUGCAGCGCCAGCAGCACCGUGAGCUACAUGCACUGGUUCCAGCAGAAGCC
CGGCCAGGCCCCCAGGAGGUGGAUCUACGACACCAGCAAGAGGGCCACCGGCGUGCCC
GCCAGGUUCAGCGGCAGCGGCAGCGGCACCGACUACACCCUGACCAUCAGCAGCCUGG
AGCCCGAGGACUUCGCCGCCUACUACUGCCAGCAGUGGACCGCCGACCCCCCCAUGCU
GACCUUCGGCGGCGGCACCAAGCUGGAGAUCAAG

The above-mentioned mRNA sequences have been generated using the first codon for each corresponding amino acid as per the standard genetic code. However, it should be noted that there exists a significant degree of degeneracy in the genetic code, implying that most amino acids (except Methionine and Tryptophan) can be coded by more than one codon. This characteristic paves the way for the creation of a multitude of mRNA sequences for any given amino acid sequence, such as those of the VH and VL chains of an antibody.

Consequently, as will be understood, alternative mRNA sequences coding for the same antibody could be generated using a different selection of codons. In practice, the generation of these alternative sequences can be guided by considerations such as the codon usage bias in the target organism (humans, in this case), the stability of the resultant mRNA, the degree of immunogenicity, and the influence of translational speed on the folding and post-translational modifications of the protein product.

Moreover, other methods, such as machine learning algorithms, could be utilized to optimize the nucleotide sequences based on these factors. These optimized sequences may provide advantages in terms of the efficiency of protein production, the stability of the mRNA, and the immunogenicity of the end product. This property of degeneracy of the genetic code, thus, underscores the potential for a large number of sequences which are within the scope of the present invention, allowing for the generation of a variety of mRNA sequences coding for the same therapeutic antibody but optimized according to different parameters.

The use of these mRNA sequences for antibody production can present numerous advantages in biomedical applications such as targeted cancer therapy, given their ability to direct the in-situ production of the antibody of interest, potentially leading to more effective, personalized treatments.

### Sequence Liabilities

As part of the present invention, we have also looked at identifying sequence liabilities (i.e. post-translational modification—PTM—sites) in the CDRs of the humanized V1 and affinity-matured clones described herein. We have identified at least one PTM site in the heavy chain and another other in the light chain. To remove the liabilities, it is proposed to introduce a single amino acid alteration in each PTM, therefore changing the CDR.

The highest risk positions have been assessed to be:

In the VL:
CDR3 position 93/94 “DP” Aspartate Fragmentation site

In the VH:
CDR2 position 53/54 “NS” Deamidation site.

This risk appears to be specific to the affinity-matured variant mAb-v53.
CDR2 position 55/56 “DG” Aspartate Isomerisation site.

Accordingly, in one aspect of the invention, any one of the L-CDR3 sequences or VL variable light chain sequences disclosed herein (either humanised or affinity-matured) may be further mutated to replace “D” (aspartic acid/aspartate) at position 93 with any one of the following amino acid residues:
A, E, F, G, H, I, K, L, N, P, Q, R, S, T, V, W, or Y.

In a further aspect, any one of the L-CDR3 sequences or VL variable light chain sequences disclosed herein (either humanised or affinity-matured) may be further mutated to replace “P” (proline) at position 94 with any one of the following amino acid residues:
A, E, F, H, I, K, L, N, Q, R, T, V, W, or Y.

In one of the changes indicated for position 93 may be paired with any one of the changes indicated for position 94.

In one preferred aspect, any one of the L-CDR3 sequences or VL variable light chain sequences disclosed herein (either humanised or affinity-matured) may be further mutated to replace the “DP” at positions 93 and 94 with any one of the following pairs of amino acid residues:
DA, DK, DN, EP, KP, NP, QP, RP, AA, EE, FF, GP, HH, II, KK, LL, NN, QQ, RR, SP, TT, WV, WW, or YY.

Thus, the VL CDR3 sequence in any of the sequences disclosed herein may be modified in the above way. Mutations DA, DK, DN, EP, KP, NP, QP, or RP may be preferred.

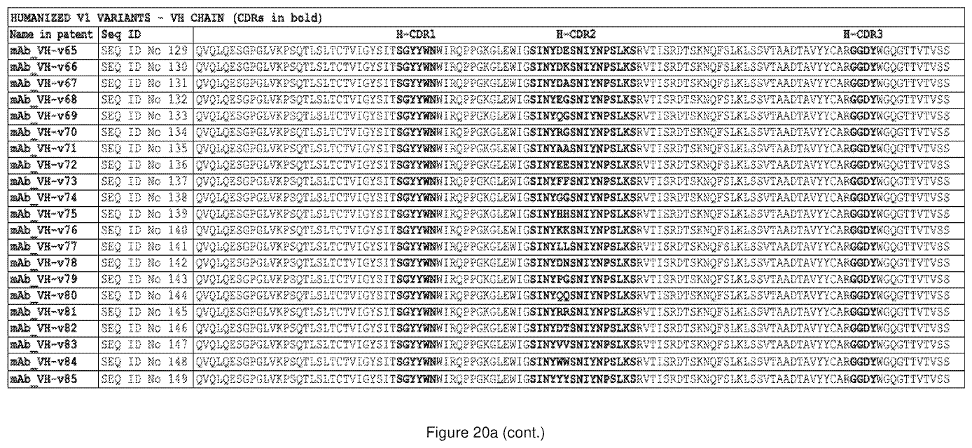

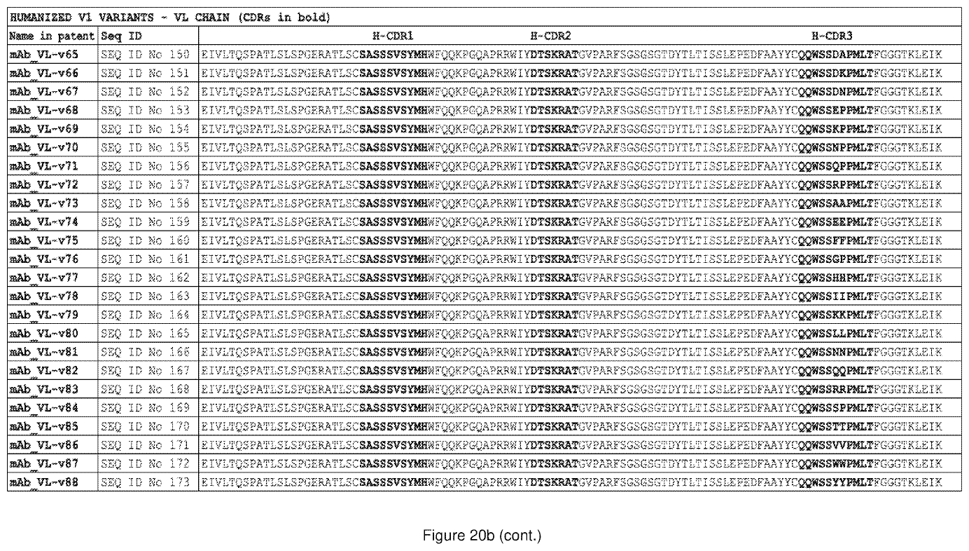

Certain preferred VL sequences incorporating the above sequence liability modifications are shown in FIG. 20b, which discloses the variable light chains of humanised variants (v65 to v88) based on V1. These are shown as SEQUENCE ID Nos 150 to 173.

Accordingly to a further aspect of the invention, any one of the H-CDR2 sequences or VH variable heavy chain sequences disclosed herein (either humanised or affinity-matured) may be further mutated to replace “D” (aspartic acid/aspartate) at position 55 with any one of the following amino acid residues:
A, E, F, G, H, I, K, L, P, Q, R, V, W or Y.

In a further aspect, any one of the H-CDR2 sequences or VH variable heavy chain sequences disclosed herein (either humanised or affinity-matured) may be further mutated to replace “G” (glycine) at position 56 with any one of the following amino acid residues:
A, E, F, H, I, K, L, N, Q, R, T, V, W or Y.

In one of the changes indicated for position 55 may be paired with any one of the changes indicated for position 56.

In one preferred aspect, any one of the H-CDR2 sequences or VH variable heavy chain sequences disclosed herein (either humanised or affinity-matured) may be further mutated to replace the “DG” at positions 55 and 56 with any one of the following pairs of amino acid residues:
DE, DK, DA, EG, QG, RG, AA, EE, FF, GG, HH, KK, LL, DN, PQ, QQ, RR, DT, WV, WW, or YY.

Thus, the VH CDR2 sequence in any of the sequences disclosed herein may be modified in the above way. Mutations DE, DK, DA, EG, QG, or RG may be preferred.

Certain preferred VH sequences incorporating the above sequence liability modifications are shown in FIG. 20a, which discloses the variable heavy chains of humanised variants (v65 to v85) based on V1. These are shown as SEQUENCE ID Nos 129 to 149.

In a preferred aspect, any one of the L-CDR3 sequences or VL variable light chain sequences disclosed herein (either humanised or affinity-matured) and containing a mutation to replace the “DP” at positions 93 and 94 with any one of the following pairs of amino acid residues:
DA, DK, DN, EP, KP, NP, QP, RP, AA, EE, FF, GP, HH, II, KK, LL, NN, QQ, RR, SP, TT, VV, WW, or YY
may be paired with any one of the H-CDR2 sequences or VH variable heavy chain sequences disclosed herein (either humanised or affinity-matured) and containing a mutation to replace the “DG” at positions 55 and 56 with any one of the following pairs of amino acid residues:
DE, DK, DA, EG, QG, RG, AA, EE, FF, GG, HH, KK, LL, DN, PQ, QQ, RR, DT, WV, WW, or YY.

In a further aspect of the invention, in the affinity-matured variant mAb-v53, it may be advantageous to further mutate this variant to replace “NS” (asparagine/serine) at position 53 and 54 of CDR2 in the variable heavy chain with alternative amino acids. For example, the N may be replaced with one of A, E, F, G, H, I, K, L, P, Q, R, V, W or Y, whilst keeping S at position 54. Alternatively, the S may be replaced with A, E, F, G, H, I, K, L, P, Q, R, V, W or Y whilst keeping N at position 54. Or both could N and S could be changed, such that a combination of the above variations may be used.

### DETAILED DESCRIPTION

### Materials and Methods

In general, where applicable, a method for the production of the antibodies of the invention can include fusion between two cells producing an hybridoma, introducing a nucleotide sequence of the invention into a host cell, culturing the host cell under suitable conditions and for a sufficient time for the production of the encoded heavy and/or light chain of the antibody or functional fragment or probe of the invention, following purification of the heavy and/light chain of an antibody or functional fragment or probe thereof.

Recombinant expression of an antibody or functional antibody fragment or probe thereof of the invention, that binds to STn or a group of alpha-2,6 sialylated antigens, can include the construction of an expression vector containing a nucleotide sequence that encodes the heavy and/or light chain of an antibody or functional antibody fragment or probe thereof of the invention.

The vector can be produced by recombinant DNA technology. Such vectors can also include other coding nucleotide sequences, originating a chimeric antibody sequence. For instance, they may include the nucleotide sequence encoding the constant region of the antibody molecule (see WO 86/05807 and WO 89701036) enabling the expression of a chimera protein, containing the amino acid sequence of the antibody, functional antibody fragment or probe thereof, of the present invention followed by the entire heavy, or light chain, or both the entire heavy and light chains of the antibody

The expression vector can be transferred to a host cell by Transfection/Transduction techniques and the resulting cells produce the antibody or functional antibody fragment thereof of the invention. Thus, the invention includes host cells containing nucleotide sequences encoding the antibody or functional antibody fragment or probe thereof of the invention.

The host cell can be chosen to modify the characteristics of the product derived from the inserted nucleotide sequences.

In one aspect, these host cells can add glycosylation or phosphorylation sites, or other modifications to the coded proteins. For example, the host cells can provide the correct processing and cell trafficking/secretion of the proteins.

Finally, in order to increase the similarity to antibodies normally produced in humans and seek to decrease immunogenicity effects, the present inventors have provided new and improved useful antibody variants, including the humanization of the parental L2A5 antibody, and throughout antibody affinity maturation of a selected humanized variant, named with the acronym of V1, leading to the generation of antibody variants with increased binding and affinity to the target STn.

Antibody clones/variants obtained were characterised in terms of binding to different cancer cell lines, specificity to the target, and immunogenicity as further described below.

In general terms, the methods employed by the inventors, and the specific processes described, in terms of their technical details, will be well understood by those skilled in this field.

### Humanization

### Variable Domain Analysis and CDR Identification

For the purpose of identifying complementarity determining regions (CDRs) and analysing the closest matching germline sequences the IMGT Domain Gap Align tool was used: http://www.imgt.org/3Dstructure-DB/cgi/DomainGapAlign.cgi

### Molecular Modelling

Molecular models were built for VH and VL domains based on homology to previously published antibody crystal structures using in-house software. PDB files allow for viewing in any molecular visualization software. Images were generated using PyMol.

### Sequence Liability Analysis

Antibody sequences were analysed for specific liabilities based on published protein motifs. Analysis was performed using an in-house system built in Microsoft Excel. The software used the following motifs where X represents any amino acid apart from Proline:

| Risk | Liability | Motif |
|---|---|---|
| High | Glycosylation | NXS or NXT |
| | Free Cysteine | C |
| | Deamidation | NG, NS or QG |
| | Isomerization | DG, DP or DS |
| Medium | Deamidation | NH |
| | Hydrolysis | NP |
| | Cleavage | TS, KK, RK or KR |
| | Transglutaminase | LLQG |
| | Integrin binding | RGD or KGD |
| | Plastic binding | FHENSP or WXXW |

### Gene Synthesis and Cloning

Variable heavy and variable light domains were designed with appropriate restriction sites at the 5′ and 3′ ends to enable cloning into Absolute Antibody cloning and expression vectors. Variable domain sequences were codon optimized for expression in human cells. Following gene synthesis the variable domains were cloned into Absolute Antibody vectors of the appropriate species and type. The correct sequence was verified by Sanger sequencing with raw data analysed using DNASTAR Lasergene software. Once confirmed plasmid DNA preparations of the appropriate size were performed to generate a sufficient quantity of high quality DNA for transfection.

### Expression and Purification

HEK 293 (human embryonic kidney 293) mammalian cells were expanded to the optimum stage for transient transfection. Cells were transiently transfected with heavy and light chain expression vectors and cultured for additional 6 days. Cultures were harvested by centrifugation at 4000 rpm, and filtered through a 0.22 M filter. A first step of purification was performed by Protein A affinity chromatography with elution using citrate pH3.0 buffer, followed by neutralization with 0.5M Tris, pH 9.0. The obtained eluted protein was then buffer exchanged into PBS, using a desalting column. Antibody concentration was determined by UV spectroscopy and the antibodies concentrated as necessary.

### Antibody Analytics

Antibody purity was determined by SDS-PAGE (sodium dodecyl sulphate polyacrylamide gel electrophoresis) and HPLC (high performance liquid chromatography). SEC-HPLC was performed on an Agilent 1100 series instrument using an appropriate size exclusion column (SEC). Antibody expression titre was determined by Protein A HPLC.

### Humanized Antibody Characterization

Different assays were performed to demonstrate whether the humanized variants maintained biophysical properties (specificity, affinity, and internalization) similar to the parental clone. These assays included:
evaluation of binding to BSM mucin (STn carrier) by ELISA
evaluation of binding to different STn+ cell lines (flow cytometry)
NMR studies to understand the antibody interaction with STn-serine glycan
in silico Immunogenicity analysis
Glycan array to determine antibody specificity
Affinity measurement by SPR
TMAs to evaluate mAb binding to patient-derived cancer tissue

### Affinity Maturation

### Library Generation

A bioinformatic analysis of the parental antibody was performed to generate a site directed CDR-mutation library. After homology modelling of the antibody Fv regions, and CDR grafting onto the template, CDR residues possibly involved in antigen binding were identified. For the heavy chain, 16 positions and for the light chain, 14 positions were identified. By analysing a NGS database, commonly used amino acids for the specific germline were identified. Based on this, degenerated codons were designed, introducing mutations at the identified position possibly involved in antigen binding. Amino acids with unfavourable characteristics were generally avoided. The introduction of mutations can be described by a gaussian distribution with an average of four mutations for each antibody chain. Primers were designed based on the degenerated codons and used for introduction of mutations into the antibody sequence. The mutated antibody genes were cloned into Yumab's scFv phage display vector and three libraries were generated and packaged into antibody-phage particles. A library with a total functional diversity greater than 5×10⁸ cfu was generated. Antibody clones with a functional open reading frame were determined by DNA sequence analysis. Packaging and purification of antibody-phage particles resulted in at least 3×10¹¹ cfu/ml for each library.

### Affinity Maturation by In-Vitro Selection

The generated antibody-phage library was used for the affinity maturation by in-vitro selection. The same overall excess of antibody-phage particles to functional size was used for each individual library. The specific amount was pooled into one library for in-vitro selection.

For the first panning round, a biotinylated BSM was used. The antibody phage output of panning round one, generated on the biotinylated protein, was used for a second round on decreasing numbers of STn+ cells to increase the stringency and drive the output towards antibodies with increased affinity. In both panning rounds a negative selection against several negative antigens was performed. Four different strategies were used for affinity maturation by in-vitro selection. By increasing the stringency from strategy one to four, a decreasing amount of eluted antibody-phage particles is expected.

### Antibody Screening

Eluted antibody-phage particles after panning round two were used for infection of E. coli. 384 clones from each strategy were selected randomly for antibody screening. In total, 1536 antibody clones were used for production of monoclonal scFv antibodies in the bacterial system. The produced antibody clones were tested for binding activity on the positive and negative cell line. The provided control antibody (IgG) as well as the parental scFv antibody were used as positive control. The parental scFv antibody was identified with a signal to noise ratio of 20, therefore clones with a signal to noise ratio greater than 20 were identified as hits.

### Antibody Sequencing

210 clones were identified as hits and selected for DNA sequence analysis. Sequence analysis revealed 40 uniquely mutated antibodies. These antibodies showed between one and six mutations in the CDR. Several hotspot mutations were identified indicating preferable mutations at different positions.

In addition, all 40 uniquely mutated antibodies were selected and soluble scFv were produced. The production was used for an ELISA screening on two positive antigens (biotinylated BSM and non-biotinylated BSM), as well as two negative antigens (streptavidin and BSA). The signal to noise ratio between positive and negative antigens was calculated for analysis. Most antibodies showed potent binding to both positive antigens and no binding to the negative antigens. Based on the generated results antibodies were selected for conversion into the final format and production in mammalian cell culture.

### Conversion to Final Format (Human IgG1):

Based on the obtained results 20 antibodies were selected for conversion into human IgG1. The antibodies were cloned into Yumab's mammalian expression vector and produced in mammalian cell culture. The antibodies were purified using protein A affinity chromatography and buffer exchanged to phosphate buffered saline. A quality control was performed by UV/VIS spectrometry and reducing SDS-PAGE. 18 antibodies were successfully produced and showed high purity and integrity. In parallel the parental antibody was cloned in the same format and produced simultaneously.

### Affinity Ranking

For validation of antibody binding, a titration on the provided positive (MDA-MB-231 STn) and negative cell lines (MDA-MB-231 WT), and also to Bovine Submaxillary Mucin (BSM), was performed. Additional titration experiments were performed with cell lines that naturally express STn (COLO205, SNU16, OV90). All antibodies showed potent and specific binding to the target cells. An EC50 value was calculated. The best antibodies showed an EC50 value around 0.6 nM, whereas the parental antibody was calculated with an EC50 value of 2 nM. The specificity of the affinity-matured antibody clones was evaluated by glycan arrays.

### Results

### Humanised Variants

### EC50 ELISA—BSM

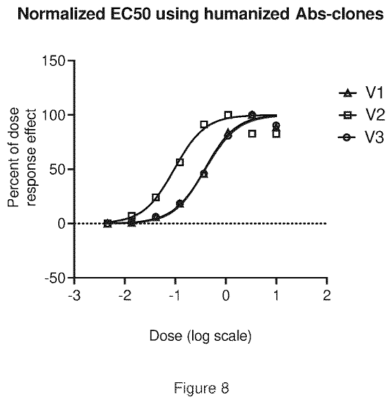

In order to evaluate the antibody binding to the target antigen STn, we employed an in vitro assay using a mucin that naturally expresses STn: Bovine Submaxillary Mucin (BSM). The humanized variants (V1, V2 and V3) were incubated using BSM-coated wells and binding was evaluated using ELISA assay. Absorbance was detected at 450 nm, and a normalized EC50 was then calculated as shown in FIG. 8.

FIG. 8 shows normalized EC50 using humanized clones V1, V2 and V3. An ELISA-assay was performed using Bovine Submaxillary Mucin (BSM). 96 well plates were coated using BSM [3 ug/mL], dissolved in 1×PBS. Absorbance was detected using a microplate reader at 450 nm. Absorbance values were normalized against the background and plotted as a % of the dose response effect versus the antibody dose (log scale) as shown in the graph. The EC50 (in ug/mL) was calculated for each condition. In one aspect, therefore, the invention thus provides humanised antibody clones which bind to the antigen STn wherein the EC50 in accordance with the test described above is less than 0.5, for example ranges between 0.05 and 0.5. For example, it may range between 0.3238 to 0.4573 for V1 clone; 0.06649 to 0.1365 for V2 clone, and 0.3440 to 0.4490 for V3 clone.

An antibody clone V2, as disclosed herein, wherein the binding affinity as measured by BSM ELISA as described herein, has a 3-fold higher percentage of dose response effect, compared to other mAb clones V1 and V3 thus suggesting better binding activity at lower concentration. Relative EC50 values are included in the FIG. 8 legend, showing a 3-fold decrease in EC50 in V2 compared to V1 and V3 clones. Together these results indicate that these humanized variants have good binding properties to the target antigen STn.

### Binding to STn+ Cell Lines

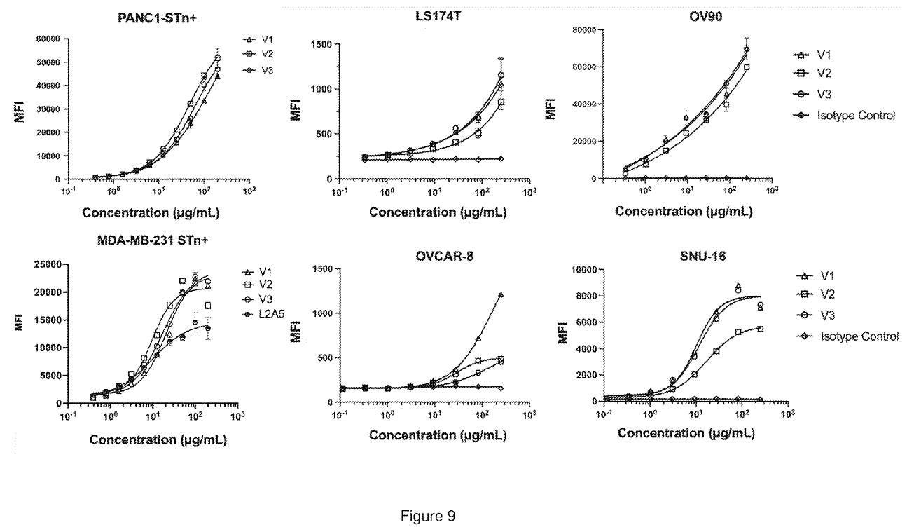

FIG. 9 shows the results of binding of humanized clones to several STn+ cell lines. Within these cell lines, four present natural expression of the STn at different levels including low (LS174T, OVCAR8), intermediate (SNU16) and high (OV90), while two cell lines presented genetic overexpression of STn (PANC1-STn and MDA-MB-231-STn). As a consequence of the different STn expression levels in those cell lines, lower EC50 (higher binding at lower dosing) was denoted in highly expressing cell lines compared to those cell lines with low STn expression. Nevertheless the results show that the three mAbs variants shown (V1, V2 and V3) maintained high binding to different cell lines expressing STn, presenting clustering to the STn antigen binding profile.

### NMR

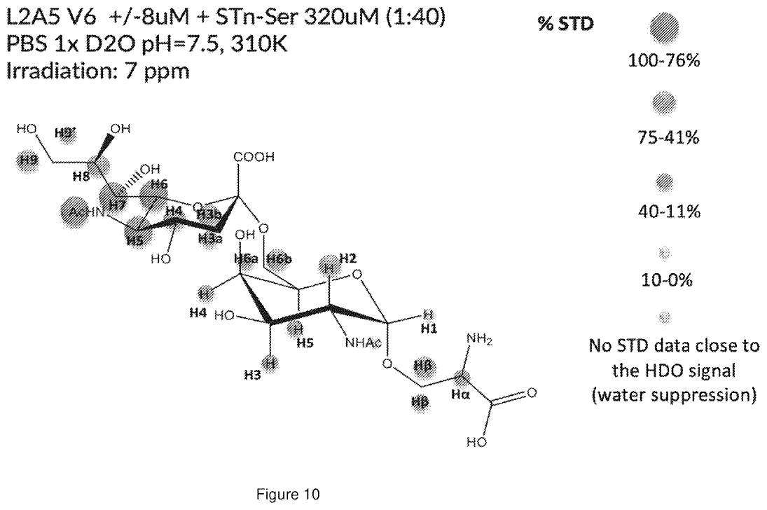

FIG. 10 illustrates how NMR studies are able to identify the points of contact/interaction between the antibody and glycan molecules. Data were acquired as saturation transfer difference (STD) using Nuclear Magnetic Resonance (NMR) to study the interaction between the ligand STn and the mAb in solution. In this experiment, using humanized variant 6 (V6), it was observed that the antibody binds mostly to the sialic acid portion of STn (bigger circles with 100-76% STD), but also interacts with the GalNac portion, according to the legend on right side of the scheme.

### SPR

FIG. 11 Summarizes Surface Plasmon Resonance (SPR) data used to measure the affinity and kinetic parameters of different anti-STn antibodies. BSA-STn was immobilized to the chip and the antibodies were injected over the surface as association phase. The humanized clones tested (V1, V2 and V3) showed a high affinity towards BSA-STn, with KD ranging from 29 nM to 39 nM. No interaction was detected between a CBS-PC clone (human positive STn control) and BSA-STn.

### Glycan Arrays

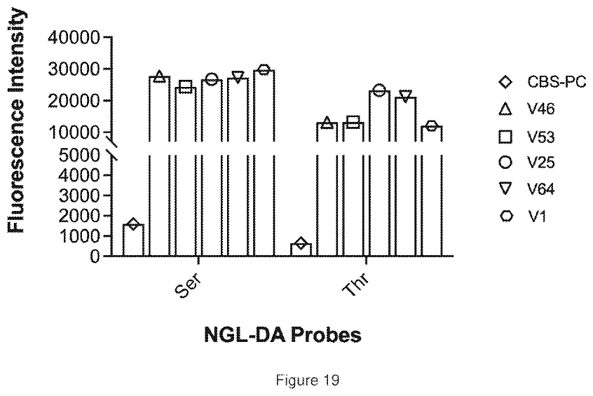

FIG. 12 and FIG. 19 illustrate that by using a chip with different immobilized glycan probes, this assay can evaluate the binding specificity and the binding intensity of antibodies to a panel of α2-6 sialylated glycans. FIG. 12A shows a 2-fold increase in binding to STn via Serine when using V1 and V3 clones compared to “positive control” mAb. FIG. 12B shows how humanized mAbs maintained specificity to STn-Ser compared to parental L2A5 mAb, confirming that the humanization process did not alter the mAb properties, while presenting increased binding to STn-Thr probes compared to parental L2A5 underlining improvement in binding to target. In addition, FIG. 19 shows how the affinity matured clones and first generation humanized clone V1 presented around a 30-fold increase in binding to STn-Ser when compared to CBS-PC control antibody. Finally, a 30-fold increase in binding to STn-Thr was denoted in V64 and V25, while a 10-fold increase was denoted in V1, V46 and V53 compared to CBS-PC control antibody. Together this data underlines the increased binding affinity of the present antibodies not only via STn-Ser but also via STn-Thr.

### In Silico Immunogenicity Analysis

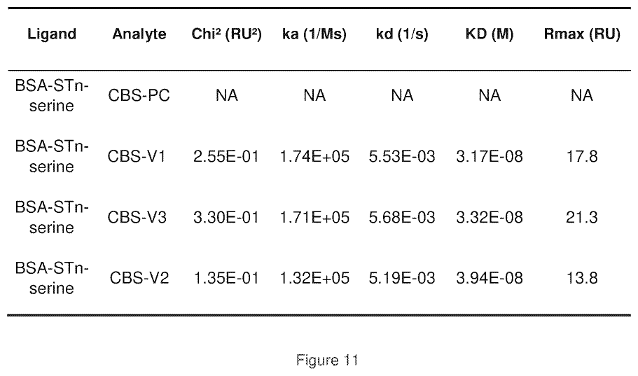

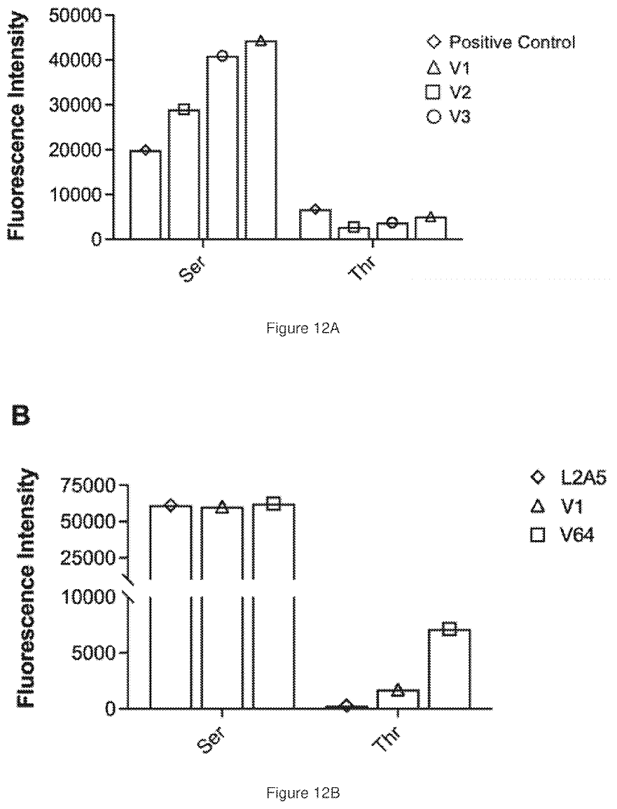

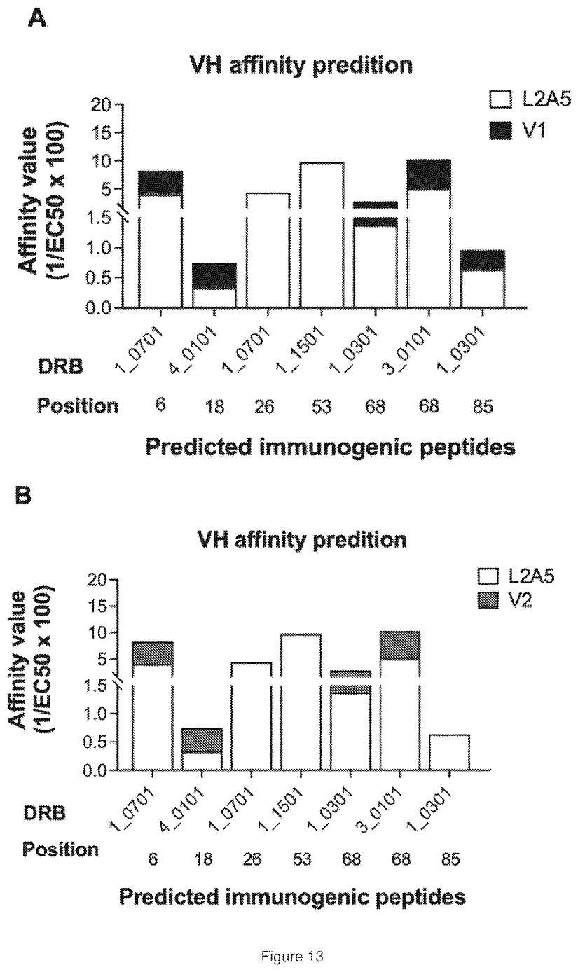

Depending on the HLA context and other factors, therapeutic antibodies may be immunogenic, inducing an immune response that may lead to premature elimination or neutralization of the molecule (human anti-mouse monoclonal antibody response, or HAMA). In order to analyze the humanized CBS variants and predict their overall immunogenicity compared to the chimeric CBS version (as previously published in WO2019147152A1), in silico tools were used to analyze the antibody sequence. As shown in FIG. 13. A-D, the results from this analysis help predict the overall immunogenicity, and we have found it can be used as a guide to further sequence optimization if necessary.

FIG. 13 (A-D) illustrates VH and VL affinity prediction in V1 and V2 compared to the Chimeric/parental L2A5 Ab. Using the NetMHCIIPan 4.0 tool parameters were set with position in protein sequence, % Rank EL (percentile rank of eluted ligand prediction score), BA (Binding Affinity), SB (Strong Binder), WB (Weak Binder). Antibody sequences were uploaded in FASTA format. Affinity values were calculated based on the Binding Affinity (BA) provided by the tool (1/EC50×100).

Results obtained from the analysis of the Variable Heavy chain (VH) indicated that both CBS-V1 and CBS-V2 had a decrease in the number of immunogenic peptides compared to the chimeric CBS (chimeric L2A5) antibody (Fig A-B). Finally, the prediction results of the Variable Light chain (VL) indicated that CBS-V1 had a decrease in the number of immunogenic peptides compared to the chimeric CBS (chimeric L2A5) antibody while CBS-V2 showed an increase in the number of immunogenic peptides compared to chimeric CBS (Fig C-D).

Using these in silico predictions, with an overall decrease of immunogenic peptides in the humanized clones V1 and V2 seen compared to the Chimeric L2A5 Ab, these results provide evidence that these variants are less immunogenic and therefore more suitable for further development, compared to chimeric CBS.

### Internalization Assay

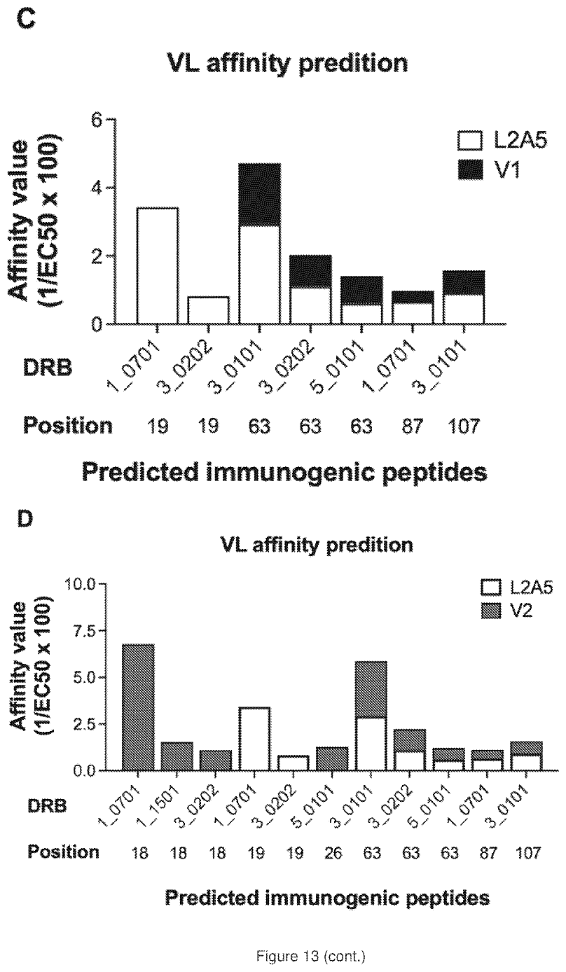

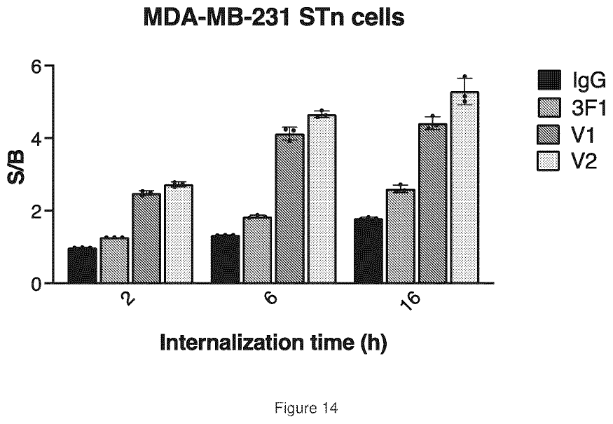

The objective of this study was to evaluate the internalization kinetics of CBS anti-STn antibodies in Breast Cancer cell lines overexpressing STn (MDA-MB231-STn). To evaluate this feature we assessed an internalization assay using humanized Abs clones (V1 and V2) together with a negative (IgG) and positive (3F1) antibodies controls as shown in FIG. 14.

FIG. 14 illustrates results from an antibody internalization-assay analysis using humanized Abs variants (V1, V2) in the Breast Cancer Cell line, MDA-MB231 overexpressing STn. 1×10⁵ target cells (MDA-MB-231 STn+) per well were plated in 96-well plates. Antibodies (10 μg/mL) were incubated for 2 h, 6 h or 16 h with the target cells (triplicates for each condition) and the pHrodo fluorescence was measured by flow cytometry, Human IgG1 [2.59 mg/ml] (Abcam, Lot. GR3218380-20) was used as a negative control. 3F1 [2.57 mg/mL], a commercial anti-STn antibody, was used as a positive control and humanized variants V1 and V2 [1 mg/ml] were tested in the assay. In this assay, the antibodies were labelled with a Zenon pHrodo fluorophore (Invitrogen, Cat.Z25611) that is activated only in low pH conditions found in the early endosome. Antibody internalization was indirectly evaluated by measuring pHrodo fluorescence by flow cytometry. A signal-to-background ratio (S/B) was calculated by dividing the MFI data from each antibody to the background (target cells only) MFI. The S/B data was plotted as mean±/−SD for each time point.

In conclusion, CBS-V1 and CBS-V2 showed higher internalization profiles in MDA-MB-231 STn+ cells when compared to 3F1 mAb. The internalization was time-dependent and showed near-peak signal at 6h, a moderate to fast internalization kinetics.

### TMAs (Tissue Microarray)

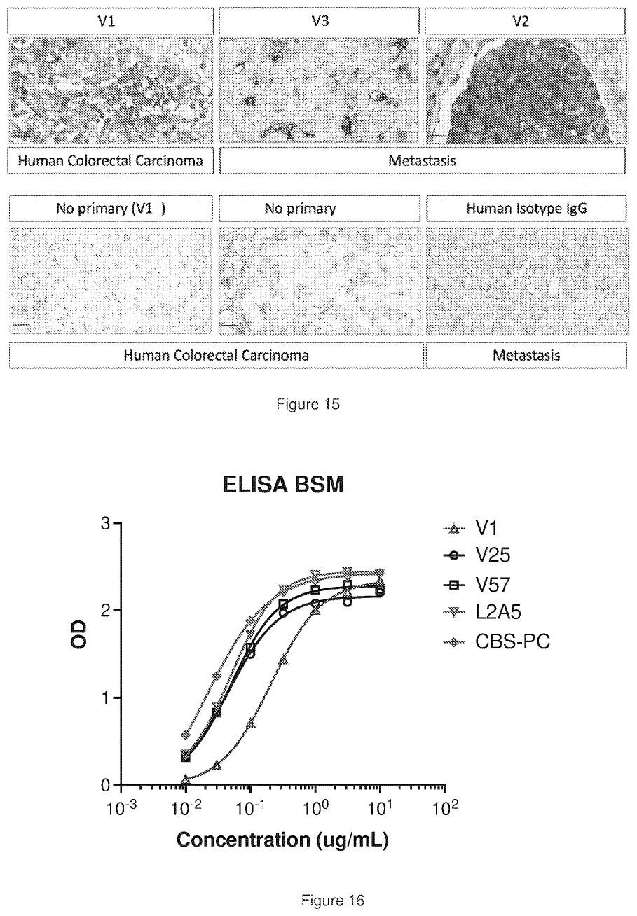

To quantitatively assess the Ab binding capacity of the newly generated humanized Ab-clones, we performed immunohistochemistry (IHC) using anti-human STn V1, V2 and V3 Abs on TMA slides. In this analysis the following Abs were tested using different TMA panels as Cancer-tumours (40 types, 95 cases) and Cancer metastasis (48 cases). Representative IHC images using V1, V2 and V3, together with the controls (no primary and isotype) are shown in FIG. 15.

FIG. 15 shows representative IHC staining using humanized V1, V2 and V3 antibody clones in colon carcinoma and different metastatic tissues. Immunohistochemistry (IHC) was performed on formalin-fixed paraffin-embedded (FFPE) human colon carcinoma tissues using a Leica Bond automated immunostainer and 3 anti-STn antibodies V1, V2 and V3 (1:5000) plus negative (no primary antibody control) and Human IgG isotype control with citrate buffer, pH 6.0. Staining was visualized in DAB (brown) and haematoxylin was used as nuclear counterstain (blue). Whole slide images were generated using a Panoramic SCAN (3D Histech). The scale bar in the figures represents 50 μm.

The results obtained using IHC together with the humanized Abs (V1, V2 and V3), strong affinity to the target antigen STn was denoted in both colorectal cancer and metastatic tissues compared to controls (no primary and human isotype). Interestingly, all the three human variants of anti-STn showed a very similar pattern of binding.

### Conclusions—Humanization

Findings and conclusions which may be drawn from the results obtained include the following:
Humanized clones derived from parental L2A5 have been found to be successfully generated
Variants V1, V2 and V3 have been shown to maintain high binding and specificity to STn
V1 and V2 are predicted to have a lower immunogenicity compared to the parental clone
We have used V1 as a starting point for an affinity maturation process, and further results from this are described below

### Affinity-Matured Clones

### Results

### EC50 ELISA—BSM

This was performed for affinity-matured clones as described above.

FIG. 16 shows EC50 ELISA results for certain clones having point mutations in the CDR regions (selected after the affinity maturation process)—these were screened against BSM mucin by ELISA. The results show that the affinity-matured variants have a lower EC50 and between a 0.5-1-fold increase in binding efficacy at lower concentration, compared to the parental V1 clone.

### EC50 Cell Lines—COLO205, SNU16, OV90, MDA-MB-231-STn

This was performed for affinity-matured clones as described above.

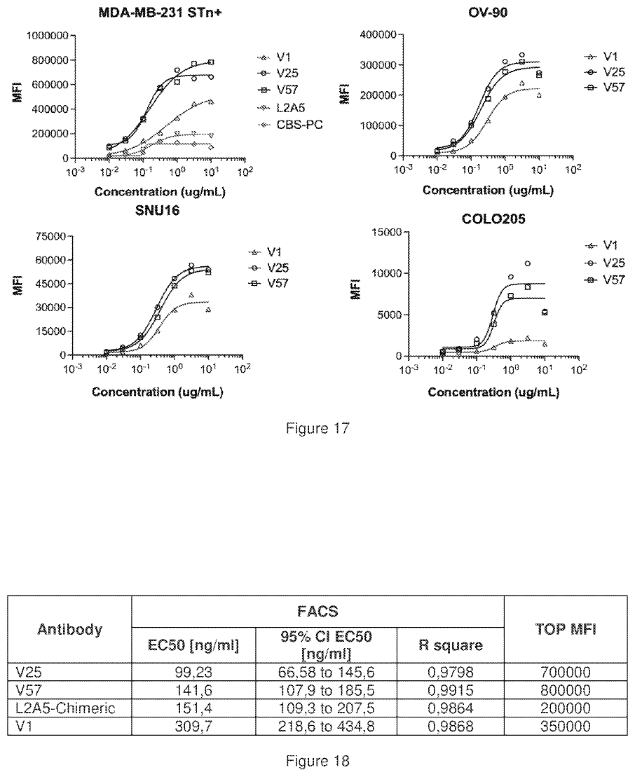

FIG. 17 shows results of the binding profile of the new affinity-matured antibody variants in cell lines with different STn expression levels. Each antibody was titrated using a 7-point concentration curve and the binding intensity was measured by flow cytometry using a secondary antibody conjugated to a fluorophore.

Based on the binding results obtained in MDA-MB-231 STn cells, the EC50 for each antibody was calculated, and these are shown in FIG. 18. The table summarizes the EC50 obtained for each clone as well as the maximum MFI detected.

In one aspect, therefore, the invention thus provides affinity-matured humanised antibody clones as described herein which bind to the antigen STn wherein the EC50 in accordance with the test described above is at least 145 ng/ml or below, preferably at least 110 ng/ml or below; more preferably at least 100 ng/ml or below, or at least 80 ng/ml or below.

### Glycan Array—Affinity-Matured Clones

This was performed for affinity-matured clones as described above.

Affinity-matured clones were evaluated in a glycan array with different sialylated probes, and the results are shown in FIG. 19. It was found that the affinity-matured clones retained specificity to STn-serine and STn-threonine (probes 3 and 4), while showing only residual binding to other probes.

### Conclusions—Affinity Maturation

Findings and conclusions which may be drawn from the results obtained include the following:
The introduction of point mutations in specific regions of V1 CDRs generated clones with higher binding affinity
Despite higher binding affinity, affinity-matured clones are still specific to STn. This is a surprising result

1. An antibody or fragment thereof which binds to the sialyl Tn (STn) antigen, wherein the antibody comprises:
(a) a heavy chain variable region (VH) wherein the VH comprises complementarity determining regions (CDRs) selected from the group consisting of:
(i) H-CDR1, H-CDR2 and H-CDR3 as shown in any one SEQ ID NOs. 1 to 24 or 49 to 88 respectively
and/or,
(b) a light chain variable region (VL) wherein the VL comprises complementarity determining regions (CDRs) selected from the group consisting of:
(i) L-CDR1, L-CDR2, and L-CDR3 as shown in any one of SEQ ID NOs. 25 to 48 or 89 to 128 respectively.

2. An antibody or fragment thereof according to claim 1, wherein the antibody comprises a heavy chain variable region (VH) and light chain variable region (VL).

3. An antibody or fragment thereof according to claim 1 or 2, wherein the antibody comprises one of the following pairs of heavy chain CDRs and light chain CDRs:
H-CDR1, H-CDR2 and H-CDR3 as shown in SEQ ID NO. 1 paired with L-CDR1, L-CDR2, and L-CDR3 as shown in SEQ ID NO. 25;
H-CDR1, H-CDR2 and H-CDR3 as shown in SEQ ID NO. 2 paired with L-CDR1, L-CDR2, and L-CDR3 as shown in SEQ ID NO. 26;
H-CDR1, H-CDR2 and H-CDR3 as shown in SEQ ID NO. 3 paired with L-CDR1, L-CDR2, and L-CDR3 as shown in SEQ ID NO. 27;
H-CDR1, H-CDR2 and H-CDR3 as shown in SEQ ID NO. 4 paired with L-CDR1, L-CDR2, and L-CDR3 as shown in SEQ ID NO. 28;
H-CDR1, H-CDR2 and H-CDR3 as shown in SEQ ID NO. 5 paired with L-CDR1, L-CDR2, and L-CDR3 as shown in SEQ ID NO. 29;
H-CDR1, H-CDR2 and H-CDR3 as shown in SEQ ID NO. 6 paired with L-CDR1, L-CDR2, and L-CDR3 as shown in SEQ ID NO. 30;
H-CDR1, H-CDR2 and H-CDR3 as shown in SEQ ID NO. 7 paired with L-CDR1, L-CDR2, and L-CDR3 as shown in SEQ ID NO. 31;
H-CDR1, H-CDR2 and H-CDR3 as shown in SEQ ID NO. 8 paired with L-CDR1, L-CDR2, and L-CDR3 as shown in SEQ ID NO. 32;
H-CDR1, H-CDR2 and H-CDR3 as shown in SEQ ID NO. 9 paired with L-CDR1, L-CDR2, and L-CDR3 as shown in SEQ ID NO. 33;
H-CDR1, H-CDR2 and H-CDR3 as shown in SEQ ID NO. 10 paired with L-CDR1, L-CDR2, and L-CDR3 as shown in SEQ ID NO. 34;
H-CDR1, H-CDR2 and H-CDR3 as shown in SEQ ID NO. 11 paired with L-CDR1, L-CDR2, and L-CDR3 as shown in SEQ ID NO. 35;
H-CDR1, H-CDR2 and H-CDR3 as shown in SEQ ID NO. 12 paired with L-CDR1, L-CDR2, and L-CDR3 as shown in SEQ ID NO. 36;
H-CDR1, H-CDR2 and H-CDR3 as shown in SEQ ID NO. 13 paired with L-CDR1, L-CDR2, and L-CDR3 as shown in SEQ ID NO. 37;
H-CDR1, H-CDR2 and H-CDR3 as shown in SEQ ID NO. 14 paired with L-CDR1, L-CDR2, and L-CDR3 as shown in SEQ ID NO. 38;
H-CDR1, H-CDR2 and H-CDR3 as shown in SEQ ID NO. 15 paired with L-CDR1, L-CDR2, and L-CDR3 as shown in SEQ ID NO. 39;
H-CDR1, H-CDR2 and H-CDR3 as shown in SEQ ID NO. 16 paired with L-CDR1, L-CDR2, and L-CDR3 as shown in SEQ ID NO. 40;
H-CDR1, H-CDR2 and H-CDR3 as shown in SEQ ID NO. 17 paired with L-CDR1, L-CDR2, and L-CDR3 as shown in SEQ ID NO. 41;
H-CDR1, H-CDR2 and H-CDR3 as shown in SEQ ID NO. 18 paired with L-CDR1, L-CDR2, and L-CDR3 as shown in SEQ ID NO. 42;
H-CDR1, H-CDR2 and H-CDR3 as shown in SEQ ID NO. 19 paired with L-CDR1, L-CDR2, and L-CDR3 as shown in SEQ ID NO. 43;
H-CDR1, H-CDR2 and H-CDR3 as shown in SEQ ID NO. 20 paired with L-CDR1, L-CDR2, and L-CDR3 as shown in SEQ ID NO. 44;
H-CDR1, H-CDR2 and H-CDR3 as shown in SEQ ID NO. 21 paired with L-CDR1, L-CDR2, and L-CDR3 as shown in SEQ ID NO. 45;
H-CDR1, H-CDR2 and H-CDR3 as shown in SEQ ID NO. 22 paired with L-CDR1, L-CDR2, and L-CDR3 as shown in SEQ ID NO. 46;
H-CDR1, H-CDR2 and H-CDR3 as shown in SEQ ID NO. 23 paired with L-CDR1, L-CDR2, and L-CDR3 as shown in SEQ ID NO. 47; or
H-CDR1, H-CDR2 and H-CDR3 as shown in SEQ ID NO. 24 paired with L-CDR1, L-CDR2, and L-CDR3 as shown in SEQ ID NO. 48.

4. An antibody or fragment thereof according to claim 3, wherein the antibody comprises one of the following pairs of heavy chain CDRs and light chain CDRs:
H-CDR1, H-CDR2 and H-CDR3 as shown in SEQ ID NO. 1 paired with L-CDR1, L-CDR2, and L-CDR3 as shown in SEQ ID NO. 25;
H-CDR1, H-CDR2 and H-CDR3 as shown in SEQ ID NO. 3 paired with L-CDR1, L-CDR2, and L-CDR3 as shown in SEQ ID NO. 27;
H-CDR1, H-CDR2 and H-CDR3 as shown in SEQ ID NO. 2 paired with L-CDR1, L-CDR2, and L-CDR3 as shown in SEQ ID NO. 26.

5. An antibody or fragment thereof according to claim 1 or 2, wherein the antibody comprises one of the following pairs of light chain CDRs and heavy chain CDRs:
H-CDR1, H-CDR2 and H-CDR3 as shown in SEQ ID NO. 49 paired with L-CDR1, L-CDR2, and L-CDR3 as shown in SEQ ID NO. 89;
H-CDR1, H-CDR2 and H-CDR3 as shown in SEQ ID NO. 50 paired with L-CDR1, L-CDR2, and L-CDR3 as shown in SEQ ID NO. 90;
H-CDR1, H-CDR2 and H-CDR3 as shown in SEQ ID NO. 51 paired with L-CDR1, L-CDR2, and L-CDR3 as shown in SEQ ID NO. 91;
H-CDR1, H-CDR2 and H-CDR3 as shown in SEQ ID NO. 52 paired with L-CDR1, L-CDR2, and L-CDR3 as shown in SEQ ID NO. 92;
H-CDR1, H-CDR2 and H-CDR3 as shown in SEQ ID NO. 53 paired with L-CDR1, L-CDR2, and L-CDR3 as shown in SEQ ID NO. 93;
H-CDR1, H-CDR2 and H-CDR3 as shown in SEQ ID NO. 54 paired with L-CDR1, L-CDR2, and L-CDR3 as shown in SEQ ID NO. 94;
H-CDR1, H-CDR2 and H-CDR3 as shown in SEQ ID NO. 55 paired with L-CDR1, L-CDR2, and L-CDR3 as shown in SEQ ID NO. 95;
H-CDR1, H-CDR2 and H-CDR3 as shown in SEQ ID NO. 56 paired with L-CDR1, L-CDR2, and L-CDR3 as shown in SEQ ID NO. 96;
H-CDR1, H-CDR2 and H-CDR3 as shown in SEQ ID NO. 57 paired with L-CDR1, L-CDR2, and L-CDR3 as shown in SEQ ID NO. 97;
H-CDR1, H-CDR2 and H-CDR3 as shown in SEQ ID NO. 58 paired with L-CDR1, L-CDR2, and L-CDR3 as shown in SEQ ID NO. 98;
H-CDR1, H-CDR2 and H-CDR3 as shown in SEQ ID NO. 59 paired with L-CDR1, L-CDR2, and L-CDR3 as shown in SEQ ID NO. 99;
H-CDR1, H-CDR2 and H-CDR3 as shown in SEQ ID NO. 60 paired with L-CDR1, L-CDR2, and L-CDR3 as shown in SEQ ID NO. 100;
H-CDR1, H-CDR2 and H-CDR3 as shown in SEQ ID NO. 61 paired with L-CDR1, L-CDR2, and L-CDR3 as shown in SEQ ID NO. 101;
H-CDR1, H-CDR2 and H-CDR3 as shown in SEQ ID NO. 62 paired with L-CDR1, L-CDR2, and L-CDR3 as shown in SEQ ID NO. 102;
H-CDR1, H-CDR2 and H-CDR3 as shown in SEQ ID NO. 63 paired with L-CDR1, L-CDR2, and L-CDR3 as shown in SEQ ID NO. 103;
H-CDR1, H-CDR2 and H-CDR3 as shown in SEQ ID NO. 64 paired with L-CDR1, L-CDR2, and L-CDR3 as shown in SEQ ID NO. 104;
H-CDR1, H-CDR2 and H-CDR3 as shown in SEQ ID NO. 65 paired with L-CDR1, L-CDR2, and L-CDR3 as shown in SEQ ID NO. 105;
H-CDR1, H-CDR2 and H-CDR3 as shown in SEQ ID NO. 66 paired with L-CDR1, L-CDR2, and L-CDR3 as shown in SEQ ID NO. 106;
H-CDR1, H-CDR2 and H-CDR3 as shown in SEQ ID NO. 67 paired with L-CDR1, L-CDR2, and L-CDR3 as shown in SEQ ID NO. 107;
H-CDR1, H-CDR2 and H-CDR3 as shown in SEQ ID NO. 68 paired with L-CDR1, L-CDR2, and L-CDR3 as shown in SEQ ID NO. 108;
H-CDR1, H-CDR2 and H-CDR3 as shown in SEQ ID NO. 69 paired with L-CDR1, L-CDR2, and L-CDR3 as shown in SEQ ID NO. 109;
H-CDR1, H-CDR2 and H-CDR3 as shown in SEQ ID NO. 70 paired with L-CDR1, L-CDR2, and L-CDR3 as shown in SEQ ID NO. 110;
H-CDR1, H-CDR2 and H-CDR3 as shown in SEQ ID NO. 71 paired with L-CDR1, L-CDR2, and L-CDR3 as shown in SEQ ID NO. 111;
H-CDR1, H-CDR2 and H-CDR3 as shown in SEQ ID NO. 72 paired with L-CDR1, L-CDR2, and L-CDR3 as shown in SEQ ID NO. 112;
H-CDR1, H-CDR2 and H-CDR3 as shown in SEQ ID NO. 73 paired with L-CDR1, L-CDR2, and L-CDR3 as shown in SEQ ID NO. 113;
H-CDR1, H-CDR2 and H-CDR3 as shown in SEQ ID NO. 74 paired with L-CDR1, L-CDR2, and L-CDR3 as shown in SEQ ID NO. 114;
H-CDR1, H-CDR2 and H-CDR3 as shown in SEQ ID NO. 75 paired with L-CDR1, L-CDR2, and L-CDR3 as shown in SEQ ID NO. 115;
H-CDR1, H-CDR2 and H-CDR3 as shown in SEQ ID NO. 76 paired with L-CDR1, L-CDR2, and L-CDR3 as shown in SEQ ID NO. 116;
H-CDR1, H-CDR2 and H-CDR3 as shown in SEQ ID NO. 77 paired with L-CDR1, L-CDR2, and L-CDR3 as shown in SEQ ID NO. 117;
H-CDR1, H-CDR2 and H-CDR3 as shown in SEQ ID NO. 78 paired with L-CDR1, L-CDR2, and L-CDR3 as shown in SEQ ID NO. 118;
H-CDR1, H-CDR2 and H-CDR3 as shown in SEQ ID NO. 79 paired with L-CDR1, L-CDR2, and L-CDR3 as shown in SEQ ID NO. 119;
H-CDR1, H-CDR2 and H-CDR3 as shown in SEQ ID NO. 80 paired with L-CDR1, L-CDR2, and L-CDR3 as shown in SEQ ID NO. 120;
H-CDR1, H-CDR2 and H-CDR3 as shown in SEQ ID NO. 81 paired with L-CDR1, L-CDR2, and L-CDR3 as shown in SEQ ID NO. 121;
H-CDR1, H-CDR2 and H-CDR3 as shown in SEQ ID NO. 82 paired with L-CDR1, L-CDR2, and L-CDR3 as shown in SEQ ID NO. 122;
H-CDR1, H-CDR2 and H-CDR3 as shown in SEQ ID NO. 83 paired with L-CDR1, L-CDR2, and L-CDR3 as shown in SEQ ID NO. 123;
H-CDR1, H-CDR2 and H-CDR3 as shown in SEQ ID NO. 84 paired with L-CDR1, L-CDR2, and L-CDR3 as shown in SEQ ID NO. 124;
H-CDR1, H-CDR2 and H-CDR3 as shown in SEQ ID NO. 85 paired with L-CDR1, L-CDR2, and L-CDR3 as shown in SEQ ID NO. 125;
H-CDR1, H-CDR2 and H-CDR3 as shown in SEQ ID NO. 86 paired with L-CDR1, L-CDR2, and L-CDR3 as shown in SEQ ID NO. 126;
H-CDR1, H-CDR2 and H-CDR3 as shown in SEQ ID NO. 87 paired with L-CDR1, L-CDR2, and L-CDR3 as shown in SEQ ID NO. 127; or
H-CDR1, H-CDR2 and H-CDR3 as shown in SEQ ID NO. 88 paired with L-CDR1, L-CDR2, and L-CDR3 as shown in SEQ ID NO. 128.

6. An antibody or fragment thereof according to claim 5, wherein the antibody comprises one of the following pairs of heavy chain CDRs and light chain CDRs:
H-CDR1, H-CDR2 and H-CDR3 as shown in SEQ ID NO. 49 paired with L-CDR1, L-CDR2, and L-CDR3 as shown in SEQ ID NO. 89;
H-CDR1, H-CDR2 and H-CDR3 as shown in SEQ ID NO. 70 paired with L-CDR1, L-CDR2, and L-CDR3 as shown in SEQ ID NO. 110;
H-CDR1, H-CDR2 and H-CDR3 as shown in SEQ ID NO. 81 paired with L-CDR1, L-CDR2, and L-CDR3 as shown in SEQ ID NO. 121; or
H-CDR1, H-CDR2 and H-CDR3 as shown in SEQ ID NO. 88 paired with L-CDR1, L-CDR2, and L-CDR3 as shown in SEQ ID NO. 128.

7. An antibody or fragment thereof according to any preceding claim, wherein the antibody comprises:
(a) a heavy chain variable region (VH) wherein the VH comprises a humanised heavy chain framework region
and/or,
(b) a light chain variable region (VL) wherein the VL comprises a humanised light chain framework region.

8. An antibody or fragment thereof according to any preceding claim, wherein the antibody comprises:
(a) a heavy chain variable region (VH) wherein the VH comprises a humanised heavy chain framework region as shown in the heavy chain variable region (VH) sequences selected from the group consisting of:
(i) any one SEQ ID NOs. 1 to 24 or 49 to 88 respectively and/or,
(b) a light chain variable region (VL) wherein the VL comprises a humanised light chain framework region as shown in the light chain variable region (VL) sequences selected from the group consisting of:
(ii) any one of SEQ ID NOs. 25 to 48 or 89 to 128 respectively.

9. An antibody or fragment thereof according to claim 7, wherein the antibody comprises one of the following pairs of light chain and heavy chain framework regions, wherein the heavy chain framework region is as shown in the heavy chain variable region (VH) sequences shown below, and wherein the light chain framework region is as shown in the light chain variable region (VL) sequences shown below:
heavy chain framework region of SEQ ID NO. 1 paired with light chain framework region from SEQ ID NO. 25;
heavy chain framework region of SEQ ID NO. 2 paired with light chain framework region from SEQ ID NO. 26;
heavy chain framework region of SEQ ID NO. 3 paired with light chain framework region from SEQ ID NO. 27;
heavy chain framework region of SEQ ID NO. 4 paired with light chain framework region from SEQ ID NO. 28;
heavy chain framework region of SEQ ID NO. 5 paired with light chain framework region from SEQ ID NO. 29;
heavy chain framework region of SEQ ID NO. 6 paired with light chain framework region from SEQ ID NO. 30;
heavy chain framework region of SEQ ID NO. 7 paired with light chain framework region from SEQ ID NO. 31;
heavy chain framework region of SEQ ID NO. 8 paired with light chain framework region from SEQ ID NO. 32;
heavy chain framework region of SEQ ID NO. 9 paired with light chain framework region from SEQ ID NO. 33;
heavy chain framework region of SEQ ID NO. 10 paired with light chain framework region from SEQ ID NO. 34;
heavy chain framework region of SEQ ID NO. 11 paired with light chain framework region from SEQ ID NO. 35;
heavy chain framework region of SEQ ID NO. 12 paired with light chain framework region from SEQ ID NO. 36;
heavy chain framework region of SEQ ID NO. 13 paired with light chain framework region from SEQ ID NO. 37;
heavy chain framework region of SEQ ID NO. 14 paired with light chain framework region from SEQ ID NO. 38;
heavy chain framework region of SEQ ID NO. 15 paired with light chain framework region from SEQ ID NO. 39;
heavy chain framework region of SEQ ID NO. 16 paired with light chain framework region from SEQ ID NO. 40;
heavy chain framework region of SEQ ID NO. 17 paired with light chain framework region from SEQ ID NO. 41;
heavy chain framework region of SEQ ID NO. 18 paired with light chain framework region from SEQ ID NO. 42;
heavy chain framework region of SEQ ID NO. 19 paired with light chain framework region from SEQ ID NO. 43;
heavy chain framework region of SEQ ID NO. 20 paired with light chain framework region from SEQ ID NO. 44;
heavy chain framework region of SEQ ID NO. 21 paired with light chain framework region from SEQ ID NO. 45;
heavy chain framework region of SEQ ID NO. 22 paired with light chain framework region from SEQ ID NO. 46;
heavy chain framework region of SEQ ID NO. 23 paired with light chain framework region from SEQ ID NO. 47; or
heavy chain framework region of SEQ ID NO. 24 paired with light chain framework region from SEQ ID NO. 48.

10. An antibody or fragment thereof according to claim 7, wherein the antibody comprises one of the following pairs of light chain and heavy chain framework regions, wherein the heavy chain framework region is as shown in the heavy chain variable region (VH) sequences shown below, and wherein the light chain framework region is as shown in the light chain variable region (VL) sequences shown below:
heavy chain framework region of SEQ ID NO. 49 paired with light chain framework region from SEQ ID NO. 89;
heavy chain framework region of SEQ ID NO. 50 paired with light chain framework region from SEQ ID NO. 90;
heavy chain framework region of SEQ ID NO. 51 paired with light chain framework region from SEQ ID NO. 91;
heavy chain framework region of SEQ ID NO. 52 paired with light chain framework region from SEQ ID NO. 92;
heavy chain framework region of SEQ ID NO. 53 paired with light chain framework region from SEQ ID NO. 93;
heavy chain framework region of SEQ ID NO. 54 paired with light chain framework region from SEQ ID NO. 94;
heavy chain framework region of SEQ ID NO. 55 paired with light chain framework region from SEQ ID NO. 95;
heavy chain framework region of SEQ ID NO. 56 paired with light chain framework region from SEQ ID NO. 96;
heavy chain framework region of SEQ ID NO. 57 paired with light chain framework region from SEQ ID NO. 97;
heavy chain framework region of SEQ ID NO. 58 paired with light chain framework region from SEQ ID NO. 98;
heavy chain framework region of SEQ ID NO. 59 paired with light chain framework region from SEQ ID NO. 99;
heavy chain framework region of SEQ ID NO. 60 paired with light chain framework region from SEQ ID NO. 100;
heavy chain framework region of SEQ ID NO. 61 paired with light chain framework region from SEQ ID NO. 101;
heavy chain framework region of SEQ ID NO. 62 paired with light chain framework region from SEQ ID NO. 102;
heavy chain framework region of SEQ ID NO. 63 paired with light chain framework region from SEQ ID NO. 103;
heavy chain framework region of SEQ ID NO. 64 paired with light chain framework region from SEQ ID NO. 104;
heavy chain framework region of SEQ ID NO. 65 paired with light chain framework region from SEQ ID NO. 105;
heavy chain framework region of SEQ ID NO. 66 paired with light chain framework region from SEQ ID NO. 106;
heavy chain framework region of SEQ ID NO. 67 paired with light chain framework region from SEQ ID NO. 107;
heavy chain framework region of SEQ ID NO. 68 paired with light chain framework region from SEQ ID NO. 108;
heavy chain framework region of SEQ ID NO. 69 paired with light chain framework region from SEQ ID NO. 109;
heavy chain framework region of SEQ ID NO. 70 paired with light chain framework region from SEQ ID NO. 110;
heavy chain framework region of SEQ ID NO. 71 paired with light chain framework region from SEQ ID NO. 111;
heavy chain framework region of SEQ ID NO. 72 paired with light chain framework region from SEQ ID NO. 112;
heavy chain framework region of SEQ ID NO. 73 paired with light chain framework region from SEQ ID NO. 113;
heavy chain framework region of SEQ ID NO. 74 paired with light chain framework region from SEQ ID NO. 114;
heavy chain framework region of SEQ ID NO. 75 paired with light chain framework region from SEQ ID NO. 115;
heavy chain framework region of SEQ ID NO. 76 paired with light chain framework region from SEQ ID NO. 116;
heavy chain framework region of SEQ ID NO. 77 paired with light chain framework region from SEQ ID NO. 117;
heavy chain framework region of SEQ ID NO. 78 paired with light chain framework region from SEQ ID NO. 118;
heavy chain framework region of SEQ ID NO. 79 paired with light chain framework region from SEQ ID NO. 119;
heavy chain framework region of SEQ ID NO. 80 paired with light chain framework region from SEQ ID NO. 120;
heavy chain framework region of SEQ ID NO. 81 paired with light chain framework region from SEQ ID NO. 121;
heavy chain framework region of SEQ ID NO. 82 paired with light chain framework region from SEQ ID NO. 122;
heavy chain framework region of SEQ ID NO. 83 paired with light chain framework region from SEQ ID NO. 123;
heavy chain framework region of SEQ ID NO. 84 paired with light chain framework region from SEQ ID NO. 124;
heavy chain framework region of SEQ ID NO. 85 paired with light chain framework region from SEQ ID NO. 125;
heavy chain framework region of SEQ ID NO. 86 paired with light chain framework region from SEQ ID NO. 126;
heavy chain framework region of SEQ ID NO. 87 paired with light chain framework region from SEQ ID NO. 127; or
heavy chain framework region of SEQ ID NO. 88 paired with light chain framework region from SEQ ID NO. 128.

11. An antibody or fragment thereof according to any one of claims 7 to 10, wherein the heavy chain framework region and/or the light chain framework region have 80% sequence identity to the sequences recited.

12. An antibody or fragment thereof according to any one of claims 7 to 11, wherein the heavy chain framework region and/or the light chain framework region have 90% sequence identity to the sequences recited.

13. An antibody or fragment thereof according to any preceding claim, wherein the antibody comprises:
(a) a heavy chain variable region (VH) selected from the group consisting of:
(i) any one SEQ ID NOs. 1 to 24 or 49 to 88 respectively
and/or,
(a) a light chain variable region (VL) selected from the group consisting of:
(ii) any one of SEQ ID NOs. 25 to 48 or 89 to 128 respectively.

14. An antibody or fragment thereof according to claim 13, wherein the antibody comprises one of the following pairs of heavy chain variable regions (VH) and light chain variable regions (VL):
SEQ ID NO. 1 paired with SEQ ID NO. 25;
SEQ ID NO. 2 paired with SEQ ID NO. 26;
SEQ ID NO. 3 paired with SEQ ID NO. 27;
SEQ ID NO. 4 paired with SEQ ID NO. 28;
SEQ ID NO. 5 paired with SEQ ID NO. 29;
SEQ ID NO. 6 paired with SEQ ID NO. 30;
SEQ ID NO. 7 paired with SEQ ID NO. 31;
SEQ ID NO. 8 paired with SEQ ID NO. 32;
SEQ ID NO. 9 paired with SEQ ID NO. 33;
SEQ ID NO. 10 paired with SEQ ID NO. 34;
SEQ ID NO. 11 paired with SEQ ID NO. 35;
SEQ ID NO. 12 paired with SEQ ID NO. 36;
SEQ ID NO. 13 paired with SEQ ID NO. 37;
SEQ ID NO. 14 paired with SEQ ID NO. 38;
SEQ ID NO. 15 paired with SEQ ID NO. 39;
SEQ ID NO. 16 paired with SEQ ID NO. 40;
SEQ ID NO. 17 paired with SEQ ID NO. 41;
SEQ ID NO. 18 paired with SEQ ID NO. 42;
SEQ ID NO. 19 paired with SEQ ID NO. 43;
SEQ ID NO. 20 paired with SEQ ID NO. 44;
SEQ ID NO. 21 paired with SEQ ID NO. 45;
SEQ ID NO. 22 paired with SEQ ID NO. 46;
SEQ ID NO. 23 paired with SEQ ID NO. 47; or
SEQ ID NO. 24 paired with SEQ ID NO. 48.

15. An antibody or fragment thereof according to claim 13, wherein the antibody comprises one of the following pairs of heavy chain variable regions (VH) and light chain variable regions (VL):
SEQ ID NO. 49 paired with SEQ ID NO. 89;
SEQ ID NO. 50 paired with SEQ ID NO. 90;
SEQ ID NO. 51 paired with SEQ ID NO. 91;
SEQ ID NO. 52 paired with SEQ ID NO. 92;
SEQ ID NO. 53 paired with SEQ ID NO. 93;
SEQ ID NO. 54 paired with SEQ ID NO. 94;
SEQ ID NO. 55 paired with SEQ ID NO. 95;
SEQ ID NO. 56 paired with SEQ ID NO. 96;
SEQ ID NO. 57 paired with SEQ ID NO. 97;
SEQ ID NO. 58 paired with SEQ ID NO. 98;
SEQ ID NO. 59 paired with SEQ ID NO. 99;
SEQ ID NO. 60 paired with SEQ ID NO. 100;
SEQ ID NO. 61 paired with SEQ ID NO. 101;
SEQ ID NO. 62 paired with SEQ ID NO. 102;
SEQ ID NO. 63 paired with SEQ ID NO. 103;
SEQ ID NO. 64 paired with SEQ ID NO. 104;
SEQ ID NO. 65 paired with SEQ ID NO. 105;
SEQ ID NO. 66 paired with SEQ ID NO. 106;
SEQ ID NO. 67 paired with SEQ ID NO. 107;
SEQ ID NO. 68 paired with SEQ ID NO. 108;
SEQ ID NO. 69 paired with SEQ ID NO. 109;
SEQ ID NO. 70 paired with SEQ ID NO. 110;
SEQ ID NO. 71 paired with SEQ ID NO. 111;
SEQ ID NO. 72 paired with SEQ ID NO. 112;
SEQ ID NO. 73 paired with SEQ ID NO. 113;
SEQ ID NO. 74 paired with SEQ ID NO. 114;
SEQ ID NO. 75 paired with SEQ ID NO. 115;
SEQ ID NO. 76 paired with SEQ ID NO. 116;
SEQ ID NO. 77 paired with SEQ ID NO. 117;
SEQ ID NO. 78 paired with SEQ ID NO. 118;
SEQ ID NO. 79 paired with SEQ ID NO. 119;
SEQ ID NO. 80 paired with SEQ ID NO. 120;
SEQ ID NO. 81 paired with SEQ ID NO. 121;
SEQ ID NO. 82 paired with SEQ ID NO. 122;
SEQ ID NO. 83 paired with SEQ ID NO. 123;
SEQ ID NO. 84 paired with SEQ ID NO. 124;
SEQ ID NO. 85 paired with SEQ ID NO. 125;
SEQ ID NO. 86 paired with SEQ ID NO. 126
SEQ ID NO. 87 paired with SEQ ID NO. 127; or
SEQ ID NO. 88 paired with SEQ ID NO. 128.

16. An antibody or fragment thereof according to any preceding claim wherein L-CDR3 is further mutated to replace D (aspartic acid/aspartate) at position 93 with any one of the following amino acid residues:
A, E, F, G, H, I, K, L, N, P, Q, R, S, T, V, W, or Y.

17. An antibody or fragment thereof according to any preceding claim wherein L-CDR3 is further mutated to replace P (proline) at position 94 with any one of the following amino acid residues:
A, E, F, H, I, K, L, N, Q, R, T, V, W, or Y.

18. An antibody or fragment thereof according to claim 16 wherein any one of the mutations indicated for position 93 is paired with any one of the mutations indicated in claim 17 for position 94.

19. An antibody or fragment thereof according to claim 16, 17 or 18, wherein L-CDR3 is further mutated to replace the DP at positions 93 and 94 with any one of the following pairs of amino acid residues:
DA, DK, DN, EP, KP, NP, QP, RP, AA, EE, FF, GP, HH, II, KK, LL, NN, QQ, RR, SP, TT, VV, WW, or YY.

20. An antibody or fragment thereof according to any one of claims 16 to 19 wherein the light chain variable sequence VL is as shown in any one of SEQUENCE ID NOs 150 to 173.

21. An antibody or fragment thereof according to any preceding claim wherein H-CDR2 is further mutated to replace D (aspartic acid/aspartate) at position 55 with any one of the following amino acid residues:
A, E, F, G, H, I, K, L, P, Q, R, V, W or Y.

22. An antibody or fragment thereof according to any preceding claim wherein H-CDR2 is further mutated to replace G (glycine) at position 56 with any one of the following amino acid residues:
A, E, F, H, I, K, L, N, Q, R, T, V, W or Y.

23. An antibody or fragment thereof according to claim 21 wherein any one of the mutations indicated for position 55 is paired with any one of the mutations indicated in claim 22 for position 56.

24. An antibody or fragment thereof according to claim 21, 22 or 23 wherein H-CDR2 is further mutated to replace the DG at positions 55 and 56 with any one of the following pairs of amino acid residues:
DE, DK, DA, EG, QG, RG, AA, EE, FF, GG, HH, KK, LL, DN, PQ, QQ, RR, DT, WV, WW, or YY.

25. An antibody or fragment thereof according to any one of claims 21 to 24 wherein the heavy chain variable sequence VH is as shown in any one of SEQUENCE ID NOs 129 to 149.

26. An antibody or fragment thereof according to any one of claims 16 to 25 wherein L-CDR3 is further mutated to replace the “DP” at positions 93 and 94 with any one of the following pairs of amino acid residues:
DA, DK, DN, EP, KP, NP, QP, RP, AA, EE, FF, GP, HH, II, KK, LL, NN, QQ, RR, SP, TT, VV, WW, or YY;
and wherein H-CDR2 is further mutated to replace the DG at positions 55 and 56 with any one of the following pairs of amino acid residues:
DE, DK, DA, EG, QG, RG, AA, EE, FF, GG, HH, KK, LL, DN, PQ, QQ, RR, DT, WV, WW, or YY.

27. An antibody or fragment thereof according to any one of claims 16 to 26 wherein L-CDR3 is further mutated to replace the “DP” at positions 93 and 94 with any one of the following pairs of amino acid residues:
DA, DK, DN, EP, KP, NP, QP, or RP;
and wherein H-CDR2 is further mutated to replace the DG at positions 55 and 56 with any one of the following pairs of amino acid residues:
DE, DK, DA, EG, QG, or RG.

28. An antibody or fragment thereof according to any one of claims 13 to 27, wherein the heavy chain variable region (VH) and/or the light chain variable region (VL) have 80% sequence identity to the sequences recited.

29. An antibody or fragment thereof according to claim 28, wherein the heavy chain variable region (VH) and/or the light chain variable region (VL) have 90% sequence identity to the sequences recited.

30. An antibody or fragment thereof according to any preceding claim that binds STn and a group of glycans terminated by alpha 2,6-linked sialic acids.

31. An antibody or fragment thereof according to claim 30 wherein the glycans terminated by alpha 2,6-linked sialic acids comprise STn, 2,6-sialyl T, di-sialyl T, or 2,6-sialolactosamine.

32. An antibody or fragment thereof according to any preceding claim that is subject to glycan changes at glycosylation sites.

33. An antibody or fragment thereof according to any preceding claim that is in the form of a ScFv (single-chain variable fragment), monoclonal antibody, chimeric antibody, humanized antibody, bispecific or multispecific antibody, an antibody drug conjugate (ADC), or cell therapies.

34. An antibody or fragment thereof according to any preceding claim that is a functional antibody fragment thereof, or a probe thereof, that binds STn and a group of glycans terminated by alpha 2,6-linked sialic acids.

35. A cell therapy composition comprising an antibody according to any preceding claim, for engaging and directing an Effector Cell to a target, wherein said Cell Therapy comprises technologies such as Chimeric Antigen Receptor (CAR) therapy, Natural Killer (NK) cell therapy, and cell-specific engagers, thereby facilitating targeted recognition and cytotoxicity against sTn-expressing cells.

36. The use of an Antibody according to any one of claims 1 to 34, which antibody is an sTn antibody, in a Cell Therapy application, wherein said sTn Antibody sequences are incorporated into Effector Cells through genetic modification, enabling specific recognition and enhanced cytotoxicity against target cells expressing sTn antigen, in conjunction with various technologies including CAR-T, NK CAR, and cell-specific engagers.

37. The use of an Antibody according to any one of claims 1 to 34, which antibody is an sTn antibody, in a Cell Therapy application for directing Effector Cells to sTn-expressing target cells, wherein said Cell Therapy comprises a range of technologies comprising, but not limited to, CAR-T, NK CAR, and cell-specific engagers, thereby promoting targeted cytotoxicity and immune-mediated responses against sTn-expressing cells.

38. The use of an Antibody according to any one of claims 1 to 34, which antibody is an sTn antibody, in a Cell Therapy application, wherein the sTn Antibody is employed to engage and direct Effector Cells through various technologies, including CAR-T, NK CAR, and cell-specific engagers, enabling targeted recognition, activation, and cytotoxicity against cells expressing the sTn antigen.

39. The use of an Antibody according to any one of claims 1 to 34, which antibody is an sTn antibody, in a Cell Therapy application for enabling targeted recognition and cytotoxicity by Effector Cells, wherein said Cell Therapy comprises a range of technologies such as CAR-T, NK CAR, and cell-specific engagers, facilitating the engagement of Effector Cells to sTn-expressing cells and promoting immune-mediated responses.

40. An antibody drug conjugate (ADC) comprising an antibody according to any preceding claim, a linker, and a cytotoxic drug.

41. A pharmaceutical composition comprising an antibody or fragment thereof according to any one of claims 1 to 34, or an antibody drug conjugate (ADC) according to claim 40, and a pharmaceutically acceptable carrier.

42. A method of detecting a tumour in a subject using an antibody or fragment thereof according to any one of claims 1 to 34, or an antibody drug conjugate (ADC) according to claim 40, or a pharmaceutical composition according to claim 41, the method comprising: staining a biological sample obtained from a subject with the nucleotide sequences encoding an antibody according to any one of claims 1 to 34, or an antibody drug conjugate (ADC) according to claim 40, under conditions suitable for specific binding of the said antibody, wherein the presence or absence of binding of the said sequences is indicative of the presence or absence of tumour cells expressing cell surface STn, 2,6-sialyl T, di-sialyl T, or 2,6-sialolactosamine, optionally wherein the biological sample comprises isolated calls, or tissue, or tumour derived proteins.

43. An antibody or fragment thereof according to any one of claims 1 to 34, or an antibody drug conjugate (ADC) according to claim 40, or a pharmaceutical composition according to claim 41, for use in medicine.

44. An antibody or fragment thereof according to any one of claims 1 to 34, or an antibody drug conjugate (ADC) according to claim 40, or a pharmaceutical composition according to claim 41, for use in treating cancer.

45. A composition comprising a nucleotide sequence encoding a monoclonal antibody or a functional antibody fragment thereof, wherein the antibody or fragment is as claimed in any one of claims 1 to 34, wherein said nucleotide sequence is encapsulated within an mRNA delivery system, wherein said mRNA delivery system is capable of facilitating transient expression of the sTn antibody or antibody fragment in a target cell.

46. A composition according to claim 45, wherein the mRNA delivery system comprises lipid-based nanoparticles, polymer-based nanoparticles, or viral vectors.

47. A method for delivering a monoclonal antibody or a functional antibody fragment thereof to a target cell, wherein the antibody or fragment is as claimed in any one of claims 1 to 34, comprising: a) encapsulating a nucleotide sequence encoding said antibody or antibody fragment within a mRNA delivery system; b) administering said mRNA delivery system to the target cell; c) allowing the mRNA to be taken up by the target cell and expressed, thereby producing the antibody or antibody fragment within the target cell.

48. A method according to claim 47, wherein the mRNA delivery system is further modified with targeting moieties to enhance specific uptake by target cells expressing the sialyl Tn (STn) antigen.

49. A therapeutic composition comprising the mRNA delivery system as described in claim 46 and a nucleotide sequence encoding a monoclonal antibody or a functional antibody fragment thereof specifically binding to the sialyl Tn (STn) antigen, wherein said composition is formulated for administration to a subject.

50. A therapeutic composition according to claim 49, further comprising a carrier or excipient suitable for systemic or localized administration to the subject.

51. A method for treating cancer characterized by the overexpression of the sialyl Tn (STn) antigen, comprising: a) the incorporation of a nucleotide sequence encoding a monoclonal antibody or a functional antibody fragment thereof binding to the STn antigen, in an mRNA delivery system; b) administering said mRNA delivery system to a subject in need thereof, thereby allowing expression of the antibody or antibody fragment within the subject's cells and selectively targeting STn-expressing cancer cells.

52. A therapeutic composition according to claim 49 or 50, or a method according to claim 51, wherein the said monoclonal antibody or a functional antibody fragment thereof comprises an antibody or fragment thereof according to any one of claims 1 to 34.

53. A theranostic composition comprising an mRNA delivery system which includes, a nucleotide sequence encoding a monoclonal antibody or a functional antibody fragment thereof according to any one of claims 1 to 34 specifically binding to the sialyl Tn (STn) antigen, and a detectable label, wherein said composition allows for both therapeutic delivery to, and imaging of, STn-expressing cancer cells.

54. A method for treating cancer in animals or humans characterized by overexpression of the sialyl Tn (STn) antigen, comprising administering to a subject in need thereof a therapeutic composition comprising an antibody or fragment thereof according to any one of claims 1 to 34, thereby selectively targeting and treating STn-expressing cancer cells in animals or humans.

55. An expression vector comprising a polynucleotide encoding an antibody according to any one of claims 1 to 34.

56. A host cell comprising an expression vector according to claim 55.

57. A method of producing an antibody or fragment thereof according to any one of claims which method comprises using a host cell according to claim 56.

58. A polynucleotide sequence encoding an antibody or a functional antibody fragment thereof, wherein the antibody or fragment thereof is as claimed in any one of claims 1 to 34.

59. A polynucleotide sequence according to claim 58 which comprises mRNA.

60. A mRNA delivery system comprising a polynucleotide according to claim 58 or 59.


SEQUENCE LISTING (178 sequences)

SEQ ID NO: 1 - protein, 113 aa, Homo sapiens
  source 1..113: note = humL2A5-v1
QVQLQESGPGLVKPSQTLSLTCTVIGYSITSGYYWNWIRQPPGKGLEWIGSINYDGSNIYNPSLKSRVTISRDTSKNQFSLKLSSVTAADTAVYYCARGGDYWGQGTTVTVSS

SEQ ID NO: 2 - protein, 113 aa, Homo sapiens
  source 1..113: note = humL2A5-v2
QVQLQESGPGLVKPSQTLSLTCSVIGYSITSGYYWNWIRQPPGKGLEWIGSINYDGSNIYNPSLKDRVTISRDTSKNQFSLKLSSVTAADTAVYYCARGGDYWGQGTTVTVSS

SEQ ID NO: 3 - protein, 113 aa, Homo sapiens; identical to SEQ ID NO: 2
  source 1..113: note = humL2A5-v3

SEQ ID NO: 4 - protein, 113 aa, Homo sapiens
  source 1..113: note = humL2A5-v4
EVQLQESGPGLVKPSQTLSLTCTVIGYSITSGYYWNWIRQPPGKGLEWMGSINYDGSNIYNPSLKSRVTISRDTSKNQFSLKLSSVTAADTAVYYCARGGDYWGQGTTVTVSS

SEQ ID NO: 5 - protein, 113 aa, Homo sapiens; identical to SEQ ID NO: 1
  source 1..113: note = humL2A5-v5

SEQ ID NO: 6 - protein, 113 aa, Homo sapiens; identical to SEQ ID NO: 1
  source 1..113: note = humL2A5-v6

SEQ ID NO: 7 - protein, 113 aa, Homo sapiens; identical to SEQ ID NO: 1
  source 1..113: note = humL2A5-v7

SEQ ID NO: 8 - protein, 113 aa, Homo sapiens; identical to SEQ ID NO: 1
  source 1..113: note = humL2A5-v8

SEQ ID NO: 9 - protein, 113 aa, Homo sapiens
  source 1..113: note = humL2A5-v9
EVQLKESGPALVKPTQTLTLTCTVIGYSITSGYYWNWIRQPPGKALEWMGSINYDGSNIYNPSLKTRLTISRDTSKNQFVLTMTNMDPVDTATYYCARGGDYWGQGTTVTVSS

SEQ ID NO: 10 - protein, 113 aa, Homo sapiens; identical to SEQ ID NO: 9
  source 1..113: note = humL2A5-v10

SEQ ID NO: 11 - protein, 113 aa, Homo sapiens; identical to SEQ ID NO: 9
  source 1..113: note = humL2A5-v11

SEQ ID NO: 12 - protein, 113 aa, Homo sapiens; identical to SEQ ID NO: 9
  source 1..113: note = humL2A5-v12

SEQ ID NO: 13 - protein, 113 aa, Homo sapiens
  source 1..113: note = humL2A5-v13
QVTLKESGPALVKPTQTLTLTCTVIGYSITSGYYWNWIRQPPGKALEWIGSINYDGSNIYNPSLKTRLTISRDTSKNQFVLTMTNMDPVDTATYYCARGGDYWGQGTTVTVSS

SEQ ID NO: 14 - protein, 113 aa, Homo sapiens; identical to SEQ ID NO: 13
  source 1..113: note = humL2A5-v14

SEQ ID NO: 15 - protein, 113 aa, Homo sapiens; identical to SEQ ID NO: 13
  source 1..113: note = humL2A5-v15

SEQ ID NO: 16 - protein, 113 aa, Homo sapiens; identical to SEQ ID NO: 13
  source 1..113: note = humL2A5-v16

SEQ ID NO: 17 - protein, 113 aa, Homo sapiens; identical to SEQ ID NO: 4
  source 1..113: note = humL2A5-v17

SEQ ID NO: 18 - protein, 113 aa, Homo sapiens; identical to SEQ ID NO: 1
  source 1..113: note = humL2A5-v18

SEQ ID NO: 19 - protein, 113 aa, Homo sapiens; identical to SEQ ID NO: 2
  source 1..113: note = humL2A5-v19

SEQ ID NO: 20 - protein, 113 aa, Homo sapiens; identical to SEQ ID NO: 4
  source 1..113: note = humL2A5-v20

SEQ ID NO: 21 - protein, 113 aa, Homo sapiens; identical to SEQ ID NO: 4
  source 1..113: note = humL2A5-v21

SEQ ID NO: 22 - protein, 113 aa, Homo sapiens
  source 1..113: note = humL2A5-v22
EVQLQESGPGLVKPSQTLSLTCSVIGYSITSGYYWNWIRQPPGKGLEWMGSINYDGSNIYNPSLKDRVTISRDTSKNQFSLKLSSVTAADTAVYYCARGGDYWGQGTSVTVSS

SEQ ID NO: 23 - protein, 113 aa, Homo sapiens; identical to SEQ ID NO: 22
  source 1..113: note = humL2A5-v23

SEQ ID NO: 24 - protein, 113 aa, Homo sapiens; identical to SEQ ID NO: 22
  source 1..113: note = humL2A5-v24

SEQ ID NO: 25 - protein, 108 aa, Homo sapiens
  source 1..108: note = humL2A5-v1
EIVLTQSPATLSLSPGERATLSCSASSSVSYMHWFQQKPGQAPRRWIYDTSKRATGVPARFSGSGSGTDYTLTISSLEPEDFAAYYCQQWSSDPPMLTFGGGTKLEIK

SEQ ID NO: 26 - protein, 108 aa, Homo sapiens
  source 1..108: note = humL2A5-v2
DIVLTQSPAILSLSPGERATLSCSASSSVSYMHWFQQKSGQAPKRWIYDTSKLASGVPARFSGSGSGTDYTLTISSLEAEDFAAYYCQQWSSDPPMLTFGGGTKLEIK

SEQ ID NO: 27 - protein, 108 aa, Homo sapiens; identical to SEQ ID NO: 25
  source 1..108: note = humL2A5-v3

SEQ ID NO: 28 - protein, 108 aa, Homo sapiens
  source 1..108: note = humL2A5-v4
DIQMTQSPSSLSASVGDRVTITCRASSSVSYMHWFQQKPGKAPKRLIYDTSKLASGVPSRFSGSGSGTDYTLTISSLQPEDFATYYCQQWSSDPPMLTFGGGTKLEIK

SEQ ID NO: 29 - protein, 108 aa, Homo sapiens
  source 1..108: note = humL2A5-v5
DIVLTQSPATLSLSPGERATLSCRASSSVSYMHWFQQKPGQAPRRLIYDTSKLASGIPARFSGSGSGTDYTLTISSLEPEDFAVYYCQQWSSDPPMLTFGGGTKLEIK

SEQ ID NO: 30 - protein, 108 aa, Homo sapiens
  source 1..108: note = humL2A5-v6
EIVLTQSPATLSLSPGERATLSCRASSSVSYMHWFQQKPGQAPRRLIYDTSKRATGIPARFSGSGSGTDYTLTISSLEPEDFAVYYCQQWSSDPPMLTFGGGTKLEIK

SEQ ID NO: 31 - protein, 108 aa, Homo sapiens
  source 1..108: note = humL2A5-v7
DIQLTQSPSSLSASVGDRVTITCRASSSVSYMHWFQQKPGKAPKRLIYDTSKLASGVPSRFSGSGSGTDYTLTISSLQPEDFATYYCQQWSSDPPMLTFGGGTKLEIK

SEQ ID NO: 32 - protein, 108 aa, Homo sapiens; identical to SEQ ID NO: 28
  source 1..108: note = humL2A5-v8

SEQ ID NO: 33 - protein, 108 aa, Homo sapiens; identical to SEQ ID NO: 29
  source 1..108: note = humL2A5-v9

SEQ ID NO: 34 - protein, 108 aa, Homo sapiens; identical to SEQ ID NO: 30
  source 1..108: note = humL2A5-v10

SEQ ID NO: 35 - protein, 108 aa, Homo sapiens; identical to SEQ ID NO: 31
  source 1..108: note = humL2A5-v11

SEQ ID NO: 36 - protein, 108 aa, Homo sapiens; identical to SEQ ID NO: 28
  source 1..108: note = humL2A5-v12

SEQ ID NO: 37 - protein, 108 aa, Homo sapiens; identical to SEQ ID NO: 29
  source 1..108: note = humL2A5-v13

SEQ ID NO: 38 - protein, 108 aa, Homo sapiens; identical to SEQ ID NO: 30
  source 1..108: note = humL2A5-v14

SEQ ID NO: 39 - protein, 108 aa, Homo sapiens; identical to SEQ ID NO: 31
  source 1..108: note = humL2A5-v15

SEQ ID NO: 40 - protein, 108 aa, Homo sapiens; identical to SEQ ID NO: 28
  source 1..108: note = humL2A5-v16

SEQ ID NO: 41 - protein, 108 aa, Homo sapiens; identical to SEQ ID NO: 29
  source 1..108: note = humL2A5-v17

SEQ ID NO: 42 - protein, 108 aa, Homo sapiens; identical to SEQ ID NO: 26
  source 1..108: note = humL2A5-v18

SEQ ID NO: 43 - protein, 108 aa, Homo sapiens; identical to SEQ ID NO: 30
  source 1..108: note = humL2A5-v19

SEQ ID NO: 44 - protein, 108 aa, Homo sapiens; identical to SEQ ID NO: 31
  source 1..108: note = humL2A5-v20

SEQ ID NO: 45 - protein, 108 aa, Homo sapiens; identical to SEQ ID NO: 30
  source 1..108: note = humL2A5-v21

SEQ ID NO: 46 - protein, 108 aa, Homo sapiens; identical to SEQ ID NO: 30
  source 1..108: note = humL2A5-v22

SEQ ID NO: 47 - protein, 108 aa, Homo sapiens; identical to SEQ ID NO: 25
  source 1..108: note = humL2A5-v23

SEQ ID NO: 48 - protein, 108 aa, Homo sapiens; identical to SEQ ID NO: 26
  source 1..108: note = humL2A5-v24

SEQ ID NO: 49 - protein, 113 aa, Homo sapiens
  source 1..113: note = YU812-A01
QVQLQESGPGLVKPSQTLSLTCTVIGQSITSGYYWNWIRQPPGKGLEWIGSINYDGSNIYNPSLKSRVTISRDTSKNQFSLKLSSVTAADTAVYYCARGGDPWGQGTTVTVSS

SEQ ID NO: 50 - protein, 113 aa, Homo sapiens
  source 1..113: note = YU812-B05
QVQLQESGPGLVKPSQTLSLTCTVIGYSITSGYYWNWIRQPPGKGLEWIGSINYDGTNIYNPSLKSRVTISRDTSKNQFSLKLSSVTAADTAVYYCARGGDPWGQGTTVTVSS

SEQ ID NO: 51 - protein, 113 aa, Homo sapiens; identical to SEQ ID NO: 1
  source 1..113: note = YU812-C09

SEQ ID NO: 52 - protein, 113 aa, Homo sapiens
  source 1..113: note = YU812-E09
QVQLQESGPGLVKPSQTLSLTCTVIGQSITAGYYWNWIRQPPGKGLEWIGSINYDGSNIYNPSLKSRVTISRDTSKNQFSLKLSSVTAADTAVYYCARGGHPWGQGTTVTVSS

SEQ ID NO: 53 - protein, 113 aa, Homo sapiens
  source 1..113: note = YU812-C02
QVQLQESGPGLVKPSQTLSLTCTVIGQSITSGYNWNWIRQPPGKGLEWIGSINHDGSNFYNPSLKSRVTISRDTSKNQFSLKLSSVTAADTAVYYCARGGAYWGQGTTVTVSS

SEQ ID NO: 54 - protein, 113 aa, Homo sapiens
  source 1..113: note = YU812-B07
QVQLQESGPGLVKPSQTLSLTCTVIGQSITSGYYWNWIRQPPGKGLEWIGSINYDGSNIYNPSLKSRVTISRDTSKNQFSLKLSSVTAADTAVYYCARGGRDWGQGTTVTVSS

SEQ ID NO: 55 - protein, 113 aa, Homo sapiens
  source 1..113: note = YU812-E04
QVQLQESGPGLVKPSQTLSLTCTVIGYSITSGYYWNWIRQPPGKGLEWIGSINYDGSNIYNPSLKSRVTISRDTSKNQFSLKLSSVTAADTAVYYCARGGDPWGQGTTVTVSS

SEQ ID NO: 56 - protein, 113 aa, Homo sapiens
  source 1..113: note = YU812-G04
QVQLQESGPGLVKPSQTLSLTCTVIGQSITSGYYWNWIRQPPGKGLEWIGSINFDGSNIYNPSLKSRVTISRDTSKNQFSLKLSSVTAADTAVYYCARGGDPWGQGTTVTVSS

SEQ ID NO: 57 - protein, 113 aa, Homo sapiens
  source 1..113: note = YU812-B09
QVQLQESGPGLVKPSQTLSLTCTVIGYSITTGYYWNWIRQPPGKGLEWIGSINYDGSNIYNPSLKSRVTISRDTSKNQFSLKLSSVTAADTAVYYCARGGEPWGQGTTVTVSS

SEQ ID NO: 58 - protein, 113 aa, Homo sapiens
  source 1..113: note = YU812-F09
QVQLQESGPGLVKPSQTLSLTCTVIGYSITQGYYWNWIRQPPGKGLEWIGSINYDGLNIYNPSLKSRVTISRDTSKNQFSLKLSSVTAADTAVYYCARGGRDWGQGTTVTVSS

SEQ ID NO: 59 - protein, 113 aa, Homo sapiens
  source 1..113: note = YU812-G05
QVQLQESGPGLVKPSQTLSLTCTVIGQSITSGYNWNWIRQPPGKGLEWIGSINYDGSNFYNPSLKSRVTISRDTSKNQFSLKLSSVTAADTAVYYCARGGNLWGQGTTVTVSS

SEQ ID NO: 60 - protein, 113 aa, Homo sapiens
  source 1..113: note = YU812-H01
QVQLQESGPGLVKPSQTLSLTCTVIGQSITAGYYWNWIRQPPGKGLEWIGSINYDGSNIYNPSLKSRVTISRDTSKNQFSLKLSSVTAADTAVYYCARGGDPWGQGTTVTVSS

SEQ ID NO: 61 - protein, 113 aa, Homo sapiens
  source 1..113: note = YU812-D10
QVQLQESGPGLVKPSQTLSLTCTVIGYSITQGYYWNWIRQPPGKGLEWIGSINYDGSNIYNPSLKSRVTISRDTSKNQFSLKLSSVTAADTAVYYCARGGDYWGQGTTVTVSS

SEQ ID NO: 62 - protein, 113 aa, Homo sapiens
  source 1..113: note = YU812-F10
QVQLQESGPGLVKPSQTLSLTCTVIGQSITSGYYWNWIRQPPGKGLEWIGSINYDGTNIYNPSLKSRVTISRDTSKNQFSLKLSSVTAADTAVYYCARGGDPWGQGTTVTVSS

SEQ ID NO: 63 - protein, 113 aa, Homo sapiens
  source 1..113: note = YU812-H02
QVQLQESGPGLVKPSQTLSLTCTVIGYSITQGYYWNWIRQPPGKGLEWIGSINYNGSNIYNPSLKSRVTISRDTSKNQFSLKLSSVTAADTAVYYCARGGDYWGQGTTVTVSS

SEQ ID NO: 64 - protein, 113 aa, Homo sapiens
  source 1..113: note = YU812-H10
QVQLQESGPGLVKPSQTLSLTCTVIGYSITSGYYWNWIRQTPGKGLEWIGSINYDGSNIYNPSLKSRVTISRDTSKNQFSLKLSSVTAADTAVYYCARGGDYWGQGTTVTVSS

SEQ ID NO: 65 - protein, 113 aa, Homo sapiens
  source 1..113: note = YU812-D03
QVQLQESGPGLVKPSQTLSLTCTVIGQSITTGYYWNWIRQPPGKGLEWIGSINYDGSNIYNPSLKSRVTISRDTSKNQFSLKLSSVTAADTAVYYCARGGDPWGQGTTVTVSS

SEQ ID NO: 66 - protein, 113 aa, Homo sapiens
  source 1..113: note = YU812-D04
QVQLQESGPGLVKPSQTLSLTCTVIGYSITQGYYWNWIRQPPGKGLEWIGSINYDGANIYNPSLKSRVTISRDTSKNQFSLKLSSVTAADTAVYYCARGGDYWGQGTTVTVSS

SEQ ID NO: 67 - protein, 113 aa, Homo sapiens
  source 1..113: note = YU813-A09
QVQLQESGPGLVKPSQTLSLTCTVIGYSITSGYYWNWIRQSPGKGLEWIGSINYDGSNIYNPSLKSRVTISRDTSKNQFSLKLSSVTAADTAVYYCARGGDYWGQGTTVTVSS

SEQ ID NO: 68 - protein, 113 aa, Homo sapiens
  source 1..113: note = YU813-B05
QVQLQESGPGLVKPSQTLSLTCTVIGYSITSSHYWNWIRQPPGKGLEWIGSINYDGSNIYNPSLKSRVTISRDTSKNQFSLKLSSVTAADTAVYYCARGGDYWGQGTTVTVSS

SEQ ID NO: 69 - protein, 113 aa, Homo sapiens; identical to SEQ ID NO: 49
  source 1..113: note = YU813-E01

SEQ ID NO: 70 - protein, 113 aa, Homo sapiens
  source 1..113: note = YU813-A10
QVQLQESGPGLVKPSQTLSLTCTVIGYSITSGYNWNWIRQPPGKGLEWIGSINPDGSNFYNPSLKSRVTISRDTSKNQFSLKLSSVTAADTAVYYCARGGDYWGQGTTVTVSS

SEQ ID NO: 71 - protein, 113 aa, Homo sapiens; identical to SEQ ID NO: 1
  source 1..113: note = YU813-B06

SEQ ID NO: 72 - protein, 113 aa, Homo sapiens; identical to SEQ ID NO: 1
  source 1..113: note = YU813-H06

SEQ ID NO: 73 - protein, 113 aa, Homo sapiens
  source 1..113: note = YU813-B07
QVQLQESGPGLVKPSQTLSLTCTVIGQSITSGYYWNWIRQPPGKGLEWIGSINYNGSNIYNPSLKSRVTISRDTSKNQFSLKLSSVTAADTAVYYCARGGDPWGQGTTVTVSS

SEQ ID NO: 74 - protein, 113 aa, Homo sapiens
  source 1..113: note = YU813-C11
QVQLQESGPGLVKPSQTLSLTCTVIGQSITSGYSWNWIRQPPGKGLEWIGSINHDGSNFYNPSLKSRVTISRDTSKNQFSLKLSSVTAADTAVYYCARGGNSWGQGTTVTVSS

SEQ ID NO: 75 - protein, 113 aa, Homo sapiens; identical to SEQ ID NO: 1
  source 1..113: note = YU813-E11

SEQ ID NO: 76 - protein, 113 aa, Homo sapiens
  source 1..113: note = YU813-A12
QVQLQESGPGLVKPSQTLSLTCTVIGYSITKGYYWNWIRQPPGKGLEWIGSINYDGSNIYNPSLKSRVTISRDTSKNQFSLKLSSVTAADTAVYYCARGGDYWGQGTTVTVSS

SEQ ID NO: 77 - protein, 113 aa, Homo sapiens
  source 1..113: note = YU813-D08
QVQLQESGPGLVKPSQTLSLTCTVIGYSITSGYSWNWIRQPPGKGLEWIGSINSDGSNFYNPSLKSRVTISRDTSKNQFSLKLSSVTAADTAVYYCARGGDYWGQGTTVTVSS

SEQ ID NO: 78 - protein, 113 aa, Homo sapiens; identical to SEQ ID NO: 1
  source 1..113: note = YU813-A05

SEQ ID NO: 79 - protein, 113 aa, Homo sapiens
  source 1..113: note = YU813-C06
QVQLQESGPGLVKPSQTLSLTCTVIGYSITSGYYWNWIRQPPGKGLEWIGSINYDGSNIYNPSLKSRVTISRDTSKNQFSLKLSSVTAADTAVYYCARGGNSWGQGTTVTVSS

SEQ ID NO: 80 - protein, 113 aa, Homo sapiens
  source 1..113: note = YU813-E06
QVQLQESGPGLVKPSQTLSLTCTVIGQSITSGYYWNWIRQPPGKGLEWIGSINYDGTNIYNPSLKSRVTISRDTSKNQFSLKLSSVTAADTAVYYCARGGRNWGQGTTVTVSS

SEQ ID NO: 81 - protein, 113 aa, Homo sapiens; identical to SEQ ID NO: 49
  source 1..113: note = YU813-H02

SEQ ID NO: 82 - protein, 113 aa, Homo sapiens
  source 1..113: note = YU813-B03
QVQLQESGPGLVKPSQTLSLTCTVIGQSITSGYYWNWIRQPPGKGLEWIGSISYDGSNIYNPSLKSRVTISRDTSKNQFSLKLSSVTAADTAVYYCARGGDPWGQGTTVTVSS

SEQ ID NO: 83 - protein, 113 aa, Homo sapiens; identical to SEQ ID NO: 64
  source 1..113: note = YU813-D11

SEQ ID NO: 84 - protein, 113 aa, Homo sapiens; identical to SEQ ID NO: 49
  source 1..113: note = YU813-H11

SEQ ID NO: 85 - protein, 113 aa, Homo sapiens
  source 1..113: note = YU813-B04
QVQLQESGPGLVKPSQTLSLTCTVIGYSITQGYYWNWIRQPPGKGLEWIGSINYDGSNIYNPSLKSRVTISRDTSKNQFSLKLSSVTAADTAVYYCARGGDPWGQGTTVTVSS

SEQ ID NO: 86 - protein, 113 aa, Homo sapiens
  source 1..113: note = YU814-A01
QVQLQESGPGLVKPSQTLSLTCTVIGYSITSGYYWNWIRQPPGKGLEWIGSINYDGTNIYNPSLKSRVTISRDTSKNQFSLKLSSVTAADTAVYYCARGGDYWGQGTTVTVSS

SEQ ID NO: 87 - protein, 113 aa, Homo sapiens
  source 1..113: note = YU814-D01
QVQLQESGPGLVKPSQTLSLTCTVIGYSITNGYYWNWIRQPPGKGLEWIGSINYDGSNIYNPSLKSRVTISRDTSKNQFSLKLSSVTAADTAVYYCAMGGAYWGQGTTVTVSS

SEQ ID NO: 88 - protein, 113 aa, Homo sapiens
  source 1..113: note = YU814-F01
QVQLQESGPGLVKPSQTLSLTCTVIGQSITSGYYWNWIRQPPGKGLEWIGSINYDGSNIYNPSLKSRVTISRDTSKNQFSLKLSSVTAADTAVYYCARGGHPWGQGTTVTVSS

SEQ ID NO: 89 - protein, 108 aa, Homo sapiens; identical to SEQ ID NO: 25
  source 1..108: note = YU812-A01

SEQ ID NO: 90 - protein, 108 aa, Homo sapiens; identical to SEQ ID NO: 25
  source 1..108: note = YU812-B05

SEQ ID NO: 91 - protein, 108 aa, Homo sapiens
  source 1..108: note = YU812-C09
EIVLTQSPATLSLSPGERATLSCSASSSVSYMHWFQQKPGQAPRRWIYDTSKRATGVPARFSGSGSGTDYTLTISSLEPEDFAAYYCQQWTADPPMLTFGGGTKLEIK

SEQ ID NO: 92 - protein, 108 aa, Homo sapiens; identical to SEQ ID NO: 25
  source 1..108: note = YU812-E09

SEQ ID NO: 93 - protein, 108 aa, Homo sapiens; identical to SEQ ID NO: 25
  source 1..108: note = YU812-C02

SEQ ID NO: 94 - protein, 108 aa, Homo sapiens; identical to SEQ ID NO: 25
  source 1..108: note = YU812-B07

SEQ ID NO: 95 - protein, 108 aa, Homo sapiens; identical to SEQ ID NO: 25
  source 1..108: note = YU812-E04

SEQ ID NO: 96 - protein, 108 aa, Homo sapiens; identical to SEQ ID NO: 25
  source 1..108: note = YU812-G04

SEQ ID NO: 97 - protein, 108 aa, Homo sapiens; identical to SEQ ID NO: 25
  source 1..108: note = YU812-B09

SEQ ID NO: 98 - protein, 108 aa, Homo sapiens; identical to SEQ ID NO: 25
  source 1..108: note = YU812-F09

SEQ ID NO: 99 - protein, 108 aa, Homo sapiens; identical to SEQ ID NO: 25
  source 1..108: note = YU812-G05

SEQ ID NO: 100 - protein, 108 aa, Homo sapiens; identical to SEQ ID NO: 25
  source 1..108: note = YU812-H01

SEQ ID NO: 101 - protein, 108 aa, Homo sapiens; identical to SEQ ID NO: 25
  source 1..108: note = YU812-D10

SEQ ID NO: 102 - protein, 108 aa, Homo sapiens; identical to SEQ ID NO: 25
  source 1..108: note = YU812-F10

SEQ ID NO: 103 - protein, 108 aa, Homo sapiens; identical to SEQ ID NO: 25
  source 1..108: note = YU812-H02

SEQ ID NO: 104 - protein, 108 aa, Homo sapiens
  source 1..108: note = YU812-H10
EIVLTQSPATLSLSPGERATLSCSASSSVSYMHWFQQKPGQAPRRWIYDTSIRATGVPARFSGSGSGTDYTLTISSLEPEDFAAYYCQQWTAHPPMLTFGGGTKLEIK

SEQ ID NO: 105 - protein, 108 aa, Homo sapiens; identical to SEQ ID NO: 25
  source 1..108: note = YU812-D03

SEQ ID NO: 106 - protein, 108 aa, Homo sapiens; identical to SEQ ID NO: 25
  source 1..108: note = YU812-D04

SEQ ID NO: 107 - protein, 108 aa, Homo sapiens
  source 1..108: note = YU813-A09
EIVLTQSPATLSLSPGERATLSCSASSTVSYMHWFQQKPGQAPRRWIYDTSKRATGVPARFSGSGSGTDYTLTISSLEPEDFAAYYCQQWTDDPPMLTFGGGTKLEIK

SEQ ID NO: 108 - protein, 108 aa, Homo sapiens; identical to SEQ ID NO: 25
  source 1..108: note = YU813-B05

SEQ ID NO: 109 - protein, 108 aa, Homo sapiens
  source 1..108: note = YU813-E01
EIVLTQSPATLSLSPGERATLSCSASSSVSHMHWFQQKPGQAPRRWIYDTSKRATGVPARFSGSGSGTDYTLTISSLEPEDFAAYYCQQWTVDPPMLTFGGGTKLEIK

SEQ ID NO: 110 - protein, 108 aa, Homo sapiens; identical to SEQ ID NO: 25
  source 1..108: note = YU813-A10

SEQ ID NO: 111 - protein, 108 aa, Homo sapiens
  source 1..108: note = YU813-B06
EIVLTQSPATLSLSPGERATLSCSASSSVAYMHWFQQKPGQAPRRWIYDTSKRATGVPARFSGSGSGTDYTLTISSLEPEDFAAYYCQQWTADPPMLTFGGGTKLEIK

SEQ ID NO: 112 - protein, 108 aa, Homo sapiens
  source 1..108: note = YU813-H06
EIVLTQSPATLSLSPGERATLSCSASSTVSYMHWFQQKPGQAPRRWIYDTSKRATGVPARFSGSGSGTDYTLTISSLEPEDFAAYYCQQWTADPPMLTFGGGTKLEIK

SEQ ID NO: 113 - protein, 108 aa, Homo sapiens; identical to SEQ ID NO: 25
  source 1..108: note = YU813-B07

SEQ ID NO: 114 - protein, 108 aa, Homo sapiens; identical to SEQ ID NO: 25
  source 1..108: note = YU813-C11

SEQ ID NO: 115 - protein, 108 aa, Homo sapiens
  source 1..108: note = YU813-E11
EIVLTQSPATLSLSPGERATLSCSASQSVSYMHWFQQKPGQAPRRWIYDTSKRATGVPARFSGSGSGTDYTLTISSLEPEDFAAYYCQQWTADPPMLTFGGGTKLEIK

SEQ ID NO: 116 - protein, 108 aa, Homo sapiens; identical to SEQ ID NO: 25
  source 1..108: note = YU813-A12

SEQ ID NO: 117 - protein, 108 aa, Homo sapiens; identical to SEQ ID NO: 25
  source 1..108: note = YU813-D08

SEQ ID NO: 118 - protein, 108 aa, Homo sapiens
  source 1..108: note = YU813-A05
EIVLTQSPATLSLSPGERATLSCSASSSVSYMHWFQQKPGQAPRRWIYDTSKRATGVPARFSGSGSGTDYTLTISSLEPEDFAAYYCQQWDADRRMLTFGGGTKLEIK

SEQ ID NO: 119 - protein, 108 aa, Homo sapiens; identical to SEQ ID NO: 25
  source 1..108: note = YU813-C06

SEQ ID NO: 120 - protein, 108 aa, Homo sapiens; identical to SEQ ID NO: 25
  source 1..108: note = YU813-E06

SEQ ID NO: 121 - protein, 108 aa, Homo sapiens
  source 1..108: note = YU813-H02
EIVLTQSPATLSLSPGERATLSCSASSSVSYMHWFQQKPGQAPRRWIYDTSKRATGVPARFSGSGSGTDYTLTISSLEPEDFAAYYCQQWSSDPPMLTFGGGTQLEIK

SEQ ID NO: 122 - protein, 108 aa, Homo sapiens; identical to SEQ ID NO: 25
  source 1..108: note = YU813-B03

SEQ ID NO: 123 - protein, 108 aa, Homo sapiens
  source 1..108: note = YU813-D11
EIVLTQSPATLSLSPGERATLSCSASSSVSYMHWFQQKPGQAPRRWIYDTSKRATGVPARFSGSGSGTDYTLTISSLEPEDFAAYYCQQWTEDPPMLTFGGGTKLEIK

SEQ ID NO: 124 - protein, 108 aa, Homo sapiens
  source 1..108: note = YU813-H11
EIVLTQSPATLSLSPGERATLSCSASSSVSYMHWFQQKPGQAPRRWIYDTSKRATGVPARFSGSGSGTDYTLTISSLEPEDFAAYYCQQWSSDPPMLTLGGGTKLEIK

SEQ ID NO: 125 - protein, 108 aa, Homo sapiens; identical to SEQ ID NO: 25
  source 1..108: note = YU813-B04

SEQ ID NO: 126 - protein, 108 aa, Homo sapiens; identical to SEQ ID NO: 25
  source 1..108: note = YU814-A01

SEQ ID NO: 127 - protein, 108 aa, Homo sapiens; identical to SEQ ID NO: 25
  source 1..108: note = YU814-D01

SEQ ID NO: 128 - protein, 108 aa, Homo sapiens; identical to SEQ ID NO: 25
  source 1..108: note = YU814-F01

SEQ ID NO: 129 - protein, 113 aa, Homo sapiens
  source 1..113: note = humL2A5-v17VH-v1
QVQLQESGPGLVKPSQTLSLTCTVIGYSITSGYYWNWIRQPPGKGLEWIGSINYDESNIYNPSLKSRVTISRDTSKNQFSLKLSSVTAADTAVYYCARGGDYWGQGTTVTVSS

SEQ ID NO: 130 - protein, 113 aa, Homo sapiens
  source 1..113: note = humL2A5-v17VH-v2
QVQLQESGPGLVKPSQTLSLTCTVIGYSITSGYYWNWIRQPPGKGLEWIGSINYDKSNIYNPSLKSRVTISRDTSKNQFSLKLSSVTAADTAVYYCARGGDYWGQGTTVTVSS

SEQ ID NO: 131 - protein, 113 aa, Homo sapiens
  source 1..113: note = humL2A5-v17VH-v3
QVQLQESGPGLVKPSQTLSLTCTVIGYSITSGYYWNWIRQPPGKGLEWIGSINYDASNIYNPSLKSRVTISRDTSKNQFSLKLSSVTAADTAVYYCARGGDYWGQGTTVTVSS

SEQ ID NO: 132 - protein, 113 aa, Homo sapiens
  source 1..113: note = humL2A5-v17VH-v4
QVQLQESGPGLVKPSQTLSLTCTVIGYSITSGYYWNWIRQPPGKGLEWIGSINYEGSNIYNPSLKSRVTISRDTSKNQFSLKLSSVTAADTAVYYCARGGDYWGQGTTVTVSS

SEQ ID NO: 133 - protein, 113 aa, Homo sapiens
  source 1..113: note = humL2A5-v17VH-v5
QVQLQESGPGLVKPSQTLSLTCTVIGYSITSGYYWNWIRQPPGKGLEWIGSINYQGSNIYNPSLKSRVTISRDTSKNQFSLKLSSVTAADTAVYYCARGGDYWGQGTTVTVSS

SEQ ID NO: 134 - protein, 113 aa, Homo sapiens
  source 1..113: note = humL2A5-v17VH-v6
QVQLQESGPGLVKPSQTLSLTCTVIGYSITSGYYWNWIRQPPGKGLEWIGSINYRGSNIYNPSLKSRVTISRDTSKNQFSLKLSSVTAADTAVYYCARGGDYWGQGTTVTVSS

SEQ ID NO: 135 - protein, 113 aa, Homo sapiens
  source 1..113: note = humL2A5-v17VH-v7
QVQLQESGPGLVKPSQTLSLTCTVIGYSITSGYYWNWIRQPPGKGLEWIGSINYAASNIYNPSLKSRVTISRDTSKNQFSLKLSSVTAADTAVYYCARGGDYWGQGTTVTVSS

SEQ ID NO: 136 - protein, 113 aa, Homo sapiens
  source 1..113: note = humL2A5-v17VH-v8
QVQLQESGPGLVKPSQTLSLTCTVIGYSITSGYYWNWIRQPPGKGLEWIGSINYEESNIYNPSLKSRVTISRDTSKNQFSLKLSSVTAADTAVYYCARGGDYWGQGTTVTVSS

SEQ ID NO: 137 - protein, 113 aa, Homo sapiens
  source 1..113: note = humL2A5-v17VH-v9
QVQLQESGPGLVKPSQTLSLTCTVIGYSITSGYYWNWIRQPPGKGLEWIGSINYFFSNIYNPSLKSRVTISRDTSKNQFSLKLSSVTAADTAVYYCARGGDYWGQGTTVTVSS

SEQ ID NO: 138 - protein, 113 aa, Homo sapiens
  source 1..113: note = humL2A5-v17VH-v10
QVQLQESGPGLVKPSQTLSLTCTVIGYSITSGYYWNWIRQPPGKGLEWIGSINYGGSNIYNPSLKSRVTISRDTSKNQFSLKLSSVTAADTAVYYCARGGDYWGQGTTVTVSS

SEQ ID NO: 139 - protein, 113 aa, Homo sapiens
  source 1..113: note = humL2A5-v17VH-v11
QVQLQESGPGLVKPSQTLSLTCTVIGYSITSGYYWNWIRQPPGKGLEWIGSINYHHSNIYNPSLKSRVTISRDTSKNQFSLKLSSVTAADTAVYYCARGGDYWGQGTTVTVSS

SEQ ID NO: 140 - protein, 113 aa, Homo sapiens
  source 1..113: note = humL2A5-v17VH-v12
QVQLQESGPGLVKPSQTLSLTCTVIGYSITSGYYWNWIRQPPGKGLEWIGSINYKKSNIYNPSLKSRVTISRDTSKNQFSLKLSSVTAADTAVYYCARGGDYWGQGTTVTVSS

SEQ ID NO: 141 - protein, 113 aa, Homo sapiens
  source 1..113: note = humL2A5-v17VH-v13
QVQLQESGPGLVKPSQTLSLTCTVIGYSITSGYYWNWIRQPPGKGLEWIGSINYLLSNIYNPSLKSRVTISRDTSKNQFSLKLSSVTAADTAVYYCARGGDYWGQGTTVTVSS

SEQ ID NO: 142 - protein, 113 aa, Homo sapiens
  source 1..113: note = humL2A5-v17VH-v14
QVQLQESGPGLVKPSQTLSLTCTVIGYSITSGYYWNWIRQPPGKGLEWIGSINYDNSNIYNPSLKSRVTISRDTSKNQFSLKLSSVTAADTAVYYCARGGDYWGQGTTVTVSS

SEQ ID NO: 143 - protein, 113 aa, Homo sapiens
  source 1..113: note = humL2A5-v17VH-v15
QVQLQESGPGLVKPSQTLSLTCTVIGYSITSGYYWNWIRQPPGKGLEWIGSINYPGSNIYNPSLKSRVTISRDTSKNQFSLKLSSVTAADTAVYYCARGGDYWGQGTTVTVSS

SEQ ID NO: 144 - protein, 113 aa, Homo sapiens
  source 1..113: note = humL2A5-v17VH-v16
QVQLQESGPGLVKPSQTLSLTCTVIGYSITSGYYWNWIRQPPGKGLEWIGSINYQQSNIYNPSLKSRVTISRDTSKNQFSLKLSSVTAADTAVYYCARGGDYWGQGTTVTVSS

SEQ ID NO: 145 - protein, 113 aa, Homo sapiens
  source 1..113: note = humL2A5-v17VH-v17
QVQLQESGPGLVKPSQTLSLTCTVIGYSITSGYYWNWIRQPPGKGLEWIGSINYRRSNIYNPSLKSRVTISRDTSKNQFSLKLSSVTAADTAVYYCARGGDYWGQGTTVTVSS

SEQ ID NO: 146 - protein, 113 aa, Homo sapiens
  source 1..113: note = humL2A5-v17VH-v18
QVQLQESGPGLVKPSQTLSLTCTVIGYSITSGYYWNWIRQPPGKGLEWIGSINYDTSNIYNPSLKSRVTISRDTSKNQFSLKLSSVTAADTAVYYCARGGDYWGQGTTVTVSS

SEQ ID NO: 147 - protein, 113 aa, Homo sapiens
  source 1..113: note = humL2A5-v17VH-v19
QVQLQESGPGLVKPSQTLSLTCTVIGYSITSGYYWNWIRQPPGKGLEWIGSINYVVSNIYNPSLKSRVTISRDTSKNQFSLKLSSVTAADTAVYYCARGGDYWGQGTTVTVSS

SEQ ID NO: 148 - protein, 113 aa, Homo sapiens
  source 1..113: note = humL2A5-v17VH-v20
QVQLQESGPGLVKPSQTLSLTCTVIGYSITSGYYWNWIRQPPGKGLEWIGSINYWWSNIYNPSLKSRVTISRDTSKNQFSLKLSSVTAADTAVYYCARGGDYWGQGTTVTVSS

SEQ ID NO: 149 - protein, 113 aa, Homo sapiens
  source 1..113: note = humL2A5-v17VH-v21
QVQLQESGPGLVKPSQTLSLTCTVIGYSITSGYYWNWIRQPPGKGLEWIGSINYYYSNIYNPSLKSRVTISRDTSKNQFSLKLSSVTAADTAVYYCARGGDYWGQGTTVTVSS

SEQ ID NO: 150 - protein, 108 aa, Homo sapiens
EIVLTQSPATLSLSPGERATLSCSASSSVSYMHWFQQKPGQAPRRWIYDTSKRATGVPARFSGSGSGTDYTLTISSLEPEDFAAYYCQQWSSDAPMLTFGGGTKLEIK

SEQ ID NO: 151 - protein, 108 aa, Homo sapiens
EIVLTQSPATLSLSPGERATLSCSASSSVSYMHWFQQKPGQAPRRWIYDTSKRATGVPARFSGSGSGTDYTLTISSLEPEDFAAYYCQQWSSDKPMLTFGGGTKLEIK

SEQ ID NO: 152 - protein, 108 aa, Homo sapiens
EIVLTQSPATLSLSPGERATLSCSASSSVSYMHWFQQKPGQAPRRWIYDTSKRATGVPARFSGSGSGTDYTLTISSLEPEDFAAYYCQQWSSDNPMLTFGGGTKLEIK

SEQ ID NO: 153 - protein, 108 aa, Homo sapiens
EIVLTQSPATLSLSPGERATLSCSASSSVSYMHWFQQKPGQAPRRWIYDTSKRATGVPARFSGSGSGTDYTLTISSLEPEDFAAYYCQQWSSEPPMLTFGGGTKLEIK

SEQ ID NO: 154 - protein, 108 aa, Homo sapiens
EIVLTQSPATLSLSPGERATLSCSASSSVSYMHWFQQKPGQAPRRWIYDTSKRATGVPARFSGSGSGTDYTLTISSLEPEDFAAYYCQQWSSKPPMLTFGGGTKLEIK

SEQ ID NO: 155 - protein, 108 aa, Homo sapiens
EIVLTQSPATLSLSPGERATLSCSASSSVSYMHWFQQKPGQAPRRWIYDTSKRATGVPARFSGSGSGTDYTLTISSLEPEDFAAYYCQQWSSNPPMLTFGGGTKLEIK

SEQ ID NO: 156 - protein, 108 aa, Homo sapiens
EIVLTQSPATLSLSPGERATLSCSASSSVSYMHWFQQKPGQAPRRWIYDTSKRATGVPARFSGSGSGTDYTLTISSLEPEDFAAYYCQQWSSQPPMLTFGGGTKLEIK

SEQ ID NO: 157 - protein, 108 aa, Homo sapiens
EIVLTQSPATLSLSPGERATLSCSASSSVSYMHWFQQKPGQAPRRWIYDTSKRATGVPARFSGSGSGTDYTLTISSLEPEDFAAYYCQQWSSRPPMLTFGGGTKLEIK

SEQ ID NO: 158 - protein, 108 aa, Homo sapiens
EIVLTQSPATLSLSPGERATLSCSASSSVSYMHWFQQKPGQAPRRWIYDTSKRATGVPARFSGSGSGTDYTLTISSLEPEDFAAYYCQQWSSAAPMLTFGGGTKLEIK

SEQ ID NO: 159 - protein, 108 aa, Homo sapiens
EIVLTQSPATLSLSPGERATLSCSASSSVSYMHWFQQKPGQAPRRWIYDTSKRATGVPARFSGSGSGTDYTLTISSLEPEDFAAYYCQQWSSEEPMLTFGGGTKLEIK

SEQ ID NO: 160 - protein, 108 aa, Homo sapiens
EIVLTQSPATLSLSPGERATLSCSASSSVSYMHWFQQKPGQAPRRWIYDTSKRATGVPARFSGSGSGTDYTLTISSLEPEDFAAYYCQQWSSFFPMLTFGGGTKLEIK

SEQ ID NO: 161 - protein, 108 aa, Homo sapiens
EIVLTQSPATLSLSPGERATLSCSASSSVSYMHWFQQKPGQAPRRWIYDTSKRATGVPARFSGSGSGTDYTLTISSLEPEDFAAYYCQQWSSGPPMLTFGGGTKLEIK

SEQ ID NO: 162 - protein, 108 aa, Homo sapiens
EIVLTQSPATLSLSPGERATLSCSASSSVSYMHWFQQKPGQAPRRWIYDTSKRATGVPARFSGSGSGTDYTLTISSLEPEDFAAYYCQQWSSHHPMLTFGGGTKLEIK

SEQ ID NO: 163 - protein, 108 aa, Homo sapiens
EIVLTQSPATLSLSPGERATLSCSASSSVSYMHWFQQKPGQAPRRWIYDTSKRATGVPARFSGSGSGTDYTLTISSLEPEDFAAYYCQQWSSIIPMLTFGGGTKLEIK

SEQ ID NO: 164 - protein, 108 aa, Homo sapiens
EIVLTQSPATLSLSPGERATLSCSASSSVSYMHWFQQKPGQAPRRWIYDTSKRATGVPARFSGSGSGTDYTLTISSLEPEDFAAYYCQQWSSKKPMLTFGGGTKLEIK

SEQ ID NO: 165 - protein, 108 aa, Homo sapiens
EIVLTQSPATLSLSPGERATLSCSASSSVSYMHWFQQKPGQAPRRWIYDTSKRATGVPARFSGSGSGTDYTLTISSLEPEDFAAYYCQQWSSLLPMLTFGGGTKLEIK

SEQ ID NO: 166 - protein, 108 aa, Homo sapiens
EIVLTQSPATLSLSPGERATLSCSASSSVSYMHWFQQKPGQAPRRWIYDTSKRATGVPARFSGSGSGTDYTLTISSLEPEDFAAYYCQQWSSNNPMLTFGGGTKLEIK

SEQ ID NO: 167 - protein, 108 aa, Homo sapiens
EIVLTQSPATLSLSPGERATLSCSASSSVSYMHWFQQKPGQAPRRWIYDTSKRATGVPARFSGSGSGTDYTLTISSLEPEDFAAYYCQQWSSQQPMLTFGGGTKLEIK

SEQ ID NO: 168 - protein, 108 aa, Homo sapiens
EIVLTQSPATLSLSPGERATLSCSASSSVSYMHWFQQKPGQAPRRWIYDTSKRATGVPARFSGSGSGTDYTLTISSLEPEDFAAYYCQQWSSRRPMLTFGGGTKLEIK

SEQ ID NO: 169 - protein, 108 aa, Homo sapiens
EIVLTQSPATLSLSPGERATLSCSASSSVSYMHWFQQKPGQAPRRWIYDTSKRATGVPARFSGSGSGTDYTLTISSLEPEDFAAYYCQQWSSSPPMLTFGGGTKLEIK

SEQ ID NO: 170 - protein, 108 aa, Homo sapiens
EIVLTQSPATLSLSPGERATLSCSASSSVSYMHWFQQKPGQAPRRWIYDTSKRATGVPARFSGSGSGTDYTLTISSLEPEDFAAYYCQQWSSTTPMLTFGGGTKLEIK

SEQ ID NO: 171 - protein, 108 aa, Homo sapiens
EIVLTQSPATLSLSPGERATLSCSASSSVSYMHWFQQKPGQAPRRWIYDTSKRATGVPARFSGSGSGTDYTLTISSLEPEDFAAYYCQQWSSVVPMLTFGGGTKLEIK

SEQ ID NO: 172 - protein, 108 aa, Homo sapiens
EIVLTQSPATLSLSPGERATLSCSASSSVSYMHWFQQKPGQAPRRWIYDTSKRATGVPARFSGSGSGTDYTLTISSLEPEDFAAYYCQQWSSWWPMLTFGGGTKLEIK

SEQ ID NO: 173 - protein, 108 aa, Homo sapiens
EIVLTQSPATLSLSPGERATLSCSASSSVSYMHWFQQKPGQAPRRWIYDTSKRATGVPARFSGSGSGTDYTLTISSLEPEDFAAYYCQQWSSYYPMLTFGGGTKLEIK

SEQ ID NO: 174 - RNA, 339 nt, Homo sapiens
  source 1..339: mol_type = mRNA
caggtgcagctgcaggagagcggccccggcctggtgaagcccagccagaccctgagcctgacctgcaccgtgatcggctacagcatcaccagcggctactactggaactggatcaggcagccccccggcaagggcctggagtggatcggcagcatcaactacgacggcagcaacatctacaaccccagcctgaagagcagggtgaccatcagcagggacaccagcaagaaccagttcagcctgaagctgagcagcgtgaccgccgccgacaccgccgtgtactactgcgccaggggcggcgactactggggccagggcaccaccgtgaccgtgagcagc

SEQ ID NO: 175 - RNA, 339 nt, Homo sapiens
  source 1..339: mol_type = mRNA
caggtgcagctgcaggagagcggccccggcctggtgaagcccagccagaccctgagcctgacctgcaccgtgatcggccagagcatcaccagcggctactactggaactggatcaggcagccccccggcaagggcctggagtggatcggcagcatcaactacgacggcagcaacatctacaaccccagcctgaagagcagggtgaccatcagcagggacaccagcaagaaccagttcagcctgaagctgagcagcgtgaccgccgccgacaccgccgtgtactactgcgccaggggcggccacccctggggccagggcaccaccgtgaccgtgagcagc

SEQ ID NO: 176 - RNA, 339 nt, Homo sapiens
  source 1..339: mol_type = mRNA
caggtgcagctgcaggagagcggccccggcctggtgaagcccagccagaccctgagcctgacctgcaccgtgatcggctacagcatcaccagcggctacaactggaactggatcaggcagccccccggcaagggcctggagtggatcggcagcatcaaccccgacggcagcaacttctacaaccccagcctgaagagcagggtgaccatcagcagggacaccagcaagaaccagttcagcctgaagctgagcagcgtgaccgccgccgacaccgccgtgtactactgcgccaggggcggcgactactggggccagggcaccaccgtgaccgtgagcagc

SEQ ID NO: 177 - RNA, 324 nt, Homo sapiens
  source 1..324: mol_type = mRNA
gagatcgtgctgacccagagccccgccaccctgagcctgagccccggcgagagggccaccctgagctgcagcgccagcagcagcgtgagctacatgcactggttccagcagaagcccggccaggcccccaggaggtggatctacgacaccagcaagagggccaccggcgtgcccgccaggttcagcggcagcggcagcggcaccgactacaccctgaccatcagcagcctggagcccgaggacttcgccgcctactactgccagcagtggagcagcgacccccccatgctgaccttcggcggcggcaccaagctggagatcaag

SEQ ID NO: 178 - RNA, 324 nt, Homo sapiens
  source 1..324: mol_type = mRNA
gagatcgtgctgacccagagccccgccaccctgagcctgagccccggcgagagggccaccctgagctgcagcgccagcagcaccgtgagctacatgcactggttccagcagaagcccggccaggcccccaggaggtggatctacgacaccagcaagagggccaccggcgtgcccgccaggttcagcggcagcggcagcggcaccgactacaccctgaccatcagcagcctggagcccgaggacttcgccgcctactactgccagcagtggaccgccgacccccccatgctgaccttcggcggcggcaccaagctggagatcaag